(19) United States
(12) Patent Application Publication
(10) Pub. No.: US 2024/0238451 A1
(43) Pub. Date: Jul. 18, 2024

(54) SINGLE-DOMAIN ANTIGEN-DEPENDENT ANTIBODY-LIKE FUSION PROTEINS

(71) Applicants: Albert Einstein College of Medicine, Bronx, NY (US); University of Helsinki, Helsingin Yliopisto (FI)

(72) Inventors: Vladislav V. Verkhusha, Bronx, NY (US); Olena S. Oliinyk, Helsinki (FI)

(21) Appl. No.: 18/561,653

(22) PCT Filed: May 26, 2022
(86) PCT No.: PCT/US22/31015

§ 371 Date: Nov. 16, 2023

Related U.S. Application Data
(60) Provisional application No. 63/194,624, filed on May 28, 2021.
(60) Provisional application No. 63/271,439, filed on Oct. 25, 2021.

(51) Int. Cl. A61K 49/00; C07K 7/06; C07K 16/10; G01N 21/64

(52) U.S. Cl. CPC A61K 49/0058; A61K 49/0045; C07K 7/06; C07K 16/1003; G01N 21/6428; C07K 2317/31; C07K 2317/569; C07K 2319/30; C07K 2319/60; G01N 2021/6439

(57) ABSTRACT

Provided herein are fusion proteins comprising a single domain antibody (sdAb) (including, but not limited to, a nanobody) that binds selectively to a specific antigen, wherein a second polypeptide is inserted into the single domain antibody, generating an internal fusion. Also provided are methods of making and methods of using the fusion proteins disclosed herein.

### STATEMENT REGARDING FEDERALLY SPONSORED RESEARCH OR DEVELOPMENT

This invention was made with U.S. government support under Federal Grant No. GM122567 awarded by the National Institutes of Health. The government has certain rights in the invention.

### INTRODUCTION

Optical imaging with NIR-FPs provides increased tissue penetration depths and better signal-to-noise ratio due to reduced light-scattering, tissue absorption and autofluorescence in NIR region (650-900 nm). NIR FPs allow labeling of whole organisms, specific cell populations, organelles, or individual proteins, and enable spectral multiplexing with FPs, biosensors and optogenetic tools active in visible range. While direct tagging of proteins with FPs ensures specificity and allows studying of protein dynamics in live cells, in some cases, FP-fusion constructs behave differently from their endogenous analogs. This might be due to altered expression level, turnover, or blocked by FP-tag functional domains.

Endogenous, not modified, proteins may be visualized by nanobodies (Nbs), which are single-domain 15 kDa antigen-binding fragments derived from camelid heavy-chain-only antibodies. Despite their small size, Nbs bind antigens with high affinity and specificity. The advantage of Nbs is their ability to recognize and bind a cognate (specific) antigen intracellularly. It has been shown that genetic fusions of Nbs with FPs of GFP-like family enable visualization of protein dynamics in live cells. However, nanobodies are usually stable irrespective of the antigen level. Accordingly, a large pool of nanobodies remains unbound to antigen. This large excess of free unbound nanobodies complicates, and in some cases, makes nearly impossible the nanobodies' use for visualization or manipulation/modification of intracellular targets.

The expression level of Nb-FP fusions may be decreased by weak or inducible promoters, or by the development of stable cell lines with low Nb-FP fusions expression. These approaches allow moderation of the impact of background signal but do not allow studies of cell populations heterogeneous by antigen expression.

To control the level of intracellular Nb-FP fusions, the use of conditionally stable Nbs containing six-point mutations in the Nb framework regions has been suggested. The diversity of Nbs is mainly determined by three complementarity determining regions (CDRs), whereas four framework regions are relatively conserved. However, the introduction of mutations at the frameworks may affect the Nb antigen-binding properties. Analysis of structures of Nb-antigen complexes shows that the considerable number of contacts with antigen is mediated by the residues in the framework regions, which limits this mutation approach to reduce intracellular level of Nbs.

Recently, an autorepression system based on the Krüppel associated box (KRAB) transcriptional repressor fused with antibody-like fibronectin-derived intrabodies that, in turn, fused to an FP, has been suggested. In these fusions, the intrabodies' expression depends on the antigen level because the unbound intrabodies fusions are translocated to the nucleus where the KRAB domain represses their transcription. However, the high accumulation of intrabodies in the nuclei (where they produce strong fluorescent signal, even if the cognate antigen is not expressed) limits this approach to non-nuclear antigens and to homogeneously antigen-expressing cell populations.

Accordingly, novel approaches for using nanobody to visualize or manipulate/modify intracellular targets are urgently needed.

The present disclosure provides fusion proteins (termed NIR-Fbs) comprising antigen-dependent near-infrared (NIR) fluorescent nanobodies that are allosterically unstable and, consequently, degrade in mammalian cells. These NIR-Fbs become highly stabilized and, consequently, brightly fluorescent, when bound to the cognate intracellular antigen. Also disclosed are methods of making and using the fusion proteins disclosed herein.

### SUMMARY

Provided herein are fusion proteins comprising a single domain antibody (sdAb) (including, but not limited to, a nanobody) that binds selectively to a specific antigen, wherein a second polypeptide is inserted into the single domain antibody, generating an internal fusion. Also provided are methods of making and methods of using the fusion proteins disclosed herein.

In one aspect, provided is an internal fusion protein comprising a nanobody, wherein a polypeptide is inserted into the nanobody. In some embodiments, the internal fusion protein comprises:
(a) a flexible linker located between the polypeptide inserted into the nanobody and the N-terminal portion of the nanobody;
(b) a flexible linker located between the polypeptide inserted into the nanobody and the C-terminal portion of the nanobody: or
(c) (i) a flexible linker located between the polypeptide inserted into the nanobody and the N-terminal portion of the nanobody and (ii) a flexible linker located between the polypeptide inserted into the nanobody and the C-terminal portion of the nanobody.

In some embodiments, the internal fusion protein comprises:
(a) a first polypeptide forming an alpha helix located between the polypeptide inserted into the nanobody and the N-terminal portion of the nanobody: and
(b) a second polypeptide forming an alpha helix located between the polypeptide inserted into the nanobody and the C-terminal portion of the nanobody.

In some embodiments the first polypeptide forming an alpha helix located comprises a sequence that is at least 90% identical to SEQ ID NO:33 and the second polypeptide forming an alpha helix comprises a sequence that is at least 90% identical to SEQ ID NO:34. In some embodiment, the first polypeptide forming an alpha helix located comprises a sequence that is at least 90% identical to SEQ ID NO:34 and the second polypeptide forming an alpha helix comprises a sequence that is at least 90% identical to SEQ ID NO:33.

In some embodiments, the first polypeptide forming an alpha helix located comprises SEQ ID NO:33 and the second polypeptide forming an alpha helix comprises SEQ ID NO:34. In some embodiments, the first polypeptide forming an alpha helix located comprises SEQ ID NO:34 and the second polypeptide forming an alpha helix comprises SEQ ID NO:33.

In some embodiments, the polypeptide is inserted into the nanobody at a position corresponding to the i) G44/K45, (ii) S65/V66, or (iii) P90/E91 insertion site in a nanobody of SEQ ID NO:8. In some embodiments, the polypeptide inserted into the nanobody is a fluorescent protein, a drug, a toxin.

In some embodiments, the polypeptide inserted into the nanobody is a non-fluorescent protein.

In some embodiments, the polypeptide inserted into the nanobody is a fluorescent protein, wherein the fluorescent protein is a near-infrared fluorescent protein. In some embodiments, the near-infrared fluorescent protein inserted into the nanobody comprises a sequence that is at least 90% identical to any one of SEQ ID NOs: 1-6. In some embodiments, the near-infrared fluorescent protein inserted into the nanobody comprises any one of SEQ ID NOs: 1-6.

In some embodiments, the polypeptide inserted into the nanobody is a toxin, wherein the toxin is caspase-3 mutant V266E, diphtheria toxin A subunit, Pseudomonas exotoxin, ricin, gelonin, or cucurmosin.

In some embodiments, the nanobody specifically binds to a fluorescent protein, a tumor antigen, or an intracellular protein.

In some embodiments the internal fusion protein is fused to a transcription factor.

In some embodiments, the polypeptide inserted into the nanobody is a kinase inhibitor. In some embodiments, the kinase inhibitor comprises the sequence GRTGRRNAI (SEQ ID NO:39) or RPKRPTTLNLF (SEQ ID NO:40).

In one aspect, provided is a multi-modular fusion protein comprising:
(a) a first internal fusion protein disclosed herein: and
(b) a second internal fusion protein disclosed herein.

In some embodiments, the nanobody of the first internal fusion protein binds to a first antigen and the nanobody of the second internal fusion protein binds to a second antigen, and wherein the first and the second antigen are different. In some embodiments, the second antigen is targeted for degradation.

Provided is an internal fusion protein or a multi-modular fusion protein, wherein the internal fusion protein or the multi-modular fusion protein comprises a signal peptide.

Provided herein are nucleic acids encoding the internal fusion proteins or the multi-modular fusion proteins disclosed herein. Provided herein are vectors comprising the nucleic acids disclosed herein. In some embodiments, the vector is a viral vector. Provided herein is a cell comprising a nucleic acid disclosed herein or a vector disclosed herein. In some embodiments, the cell is an isolated cell.

In one aspect, provided is a method of recoloring a cell expressing a first fluorescent protein, the method comprising:
(a) expressing an internal fusion protein disclosed herein in the cell, wherein a second fluorescent protein is inserted into the nanobody and wherein the nanobody binds to the first fluorescent protein;
(b) detecting the fluorescence of the second fluorescent protein.

In one aspect, provided is a method of detecting the simultaneous presence of a first and a second antigen in a cell, the method comprising:
(a) expressing in the cell a first internal fusion protein disclosed herein fused to a second internal fusion protein disclosed herein: wherein the first internal fusion protein comprises (i) a first nanobody that binds to the first antigen and (ii) a first fluorescent protein inserted into the first nanobody: and wherein the second internal fusion protein comprises (i) a second nanobody that binds to the second antigen and optionally (ii) a second fluorescent protein inserted into the second nanobody: and
(b) detecting the fluorescence of the first and optionally the fluorescence of the second fluorescent protein: wherein presence of fluorescence of the first and optionally of the second fluorescent protein indicates simultaneous presence of the first and the second antigen in the cell: and absence of fluorescence of the first and optionally of the second fluorescent protein indicates absence of (i) the first antigen, (ii), the second antigen, or (iii) the first and the second antigen in the cell.

In one aspect, provided is a method of promoting degradation of a target antigen in a cell, the method comprising expressing in the cell an internal fusion protein disclosed herein comprising a first nanobody directed against a first antigen, wherein the internal fusion protein is fused to second nanobody directed against a second antigen that is to be targeted for degradation and wherein (a) in the presence of the first antigen, degradation of the second antigen is not promoted: and (b) in the absence of the first antigen, degradation of the second antigen is promoted. In some embodiments, a fluorescent protein is inserted into the first nanobody, and wherein (a) in the presence of the first antigen, the fluorescent protein emits fluorescence: and (b) in the absence of the first antigen, the fluorescent protein does not emit fluorescence.

In one aspect, provided is a method of regulating the expression of a protein of interest in a cell, the method comprising expressing in the cell an internal fusion protein disclosed herein comprising a nanobody directed against an antigen, wherein the internal fusion protein is fused to transcription factor that controls the expressing of the protein of interest, wherein (a) in the presence of the antigen, expression of the protein of interest is increased: and (b) in the absence of the antigen, expression of the protein of interest is decreased. In some embodiments, a fluorescent protein is inserted into the first nanobody, and wherein (a) in the presence of the antigen, the fluorescent protein emits fluorescence: and (b) in the absence of the antigen, the fluorescent protein does not emit fluorescence.

In one aspect, provided is a method of regulating kinase activity in a cell, the method comprising expressing in the cell an internal fusion protein disclosed herein comprising a nanobody directed against an antigen, wherein the internal fusion protein is fused to a peptide that reduces the activity of a kinase and wherein (a) in the presence of the antigen, the activity of the kinase is reduced; and (b) in the absence of the antigen, the activity of the kinase is not reduced. In some embodiments, a fluorescent protein is inserted into the first nanobody, and wherein (a) in the presence of the antigen, the fluorescent protein emits fluorescence; and (b) in the absence of the antigen, the fluorescent protein does not emit fluorescence.

### BRIEF DESCRIPTION OF THE DRAWINGS

FIGS. 1A and 1B illustrate engineering of red-shifted miRFPnano variants. FIG. 1A. Excitation and emission spectra of miRFP670nano and miRFP670nano/R57C/C86S mutant. The excitation and emission spectra of miRFP670nano/R57C/C86S mutant are redshifted as compared to miRFP670nano. FIG. 1B. Excitation and emission spectra of miRFP670nano/R57C/C86S and miRFP670nano/R57C/C86S/L114F/V115S mutants.

FIGS. 2A, 2B, 2C, 2D, 2E, 2F, 2G, 2H, 2I, 2J, 2K, and 2L. Characterization of miRFPnano proteins. FIG. 2A. Absorbance spectra. FIG. 2B. Fluorescence excitation spectra. FIG. 2C. Fluorescence emission spectra. FIG. 2D. Excitation and emission spectra of miRFP670nano3 with biliverdin (BV) and with phycocyanobilin (PCB) chromophore. FIG. 2E, pH dependencies of fluorescence for miRFP670nano3, miRFP704nano, and miRFP718nano in comparison with parental miRFP670nano. FIG. 2F, pH dependencies of fluorescence for miRFP670nano3 with BV and with PCB chromophore. FIG. 2G. Effective (cellular) brightness of miRFP670nano3, miRFP704nano, and miRFP718nano in transiently transfected HeLa. N2A (mouse neuroblastoma cell line). U-2 OS (human osteosarcoma cells). HEK293T (human kidney cells), and NIH3T3 (murine fibroblast cells) live mammalian cells. Fluorescence intensity was analyzed by flow cytometry 72 h after transfection and normalized to the excitation efficiency of each miRFPnano by 640 nm laser and the emission spectrum of each miRFPnano in the emission filter. The effective brightness of miRFP670nano was assumed to 100% for each cell type. Error bars, s.d. (n=3; transfection experiments). FIG. 2H. Photobleaching kinetics of miRFP670nano3, miRFP704nano, and miRFP718nano in comparison with parental miRFP670nano in live HeLa cells. FIG. 2I. Fluorescence intensity of live HeLa cells transiently transfected with miRFP670nano3, miRFP704nano, miRFP718nano, miRFP670nano, or EGFP before and after 4 h of incubation with 20 μg/ml cycloheximide. Error bars, s.d. (n=3; transfection experiments). FIG. 2J. Fluorescence intensity of live HeLa cells transiently transfected with miRFP670nano3, miRFP704nano, miRFP718nano, miRFP670nano, or EGFP before and after 4 h of incubation with 10 μM bortezomib. Error bars, s.d. (n=3 transfection experiments). FIG. 2K. Stability of miRFPnanos in Hela cells. The number of FP expressing (fluorescent) HeLa cells transiently transfected with parental miRFP670nano, miRFP670nano3, miRFP704nano, miRFP718nano or EGFP was calculated 48 h and 120 h after transfection. The values were normalized to the number of cells observed 48 h after transfection. Error bars, s.d. (n=3 transfection experiments). FIG. 2L. Fluorescence intensity of live Hela cells transiently transfected with miRFP670nano3, miRFP704nano, miRFP718nano, miRFP670nano, or EGFP 48 h and 120 h after transfection normalized to that at 48 h. Error bars, s.d. (n=5 transfection experiments).

FIGS. 3A, 3B, 3C, 3D, 3E, 3F, 3G, and 3H. Protein fusions of miRFPnanos imaged using epifluorescence microscopy. HeLa cells transiently transfected with miRFP670nano3 (FIG. 3A), miRFP704nano (FIG. 3B), and miRFP718nano (FIG. 3C) N- and C-terminal fusion constructs. The N-terminal fusions are σ-tubulin, β-actin, myosin and vesicular protein clathrin. The C-terminal fusions are lysosomal membrane glycoprotein LAMP1 and histone H2B. Scale bars. 10 μm. FIG. 3D. Two-color images of HeLa cells co-expressing a-tubulin tagged with miRFP670nano3 and H2B tagged with miRFP718nano. FIG. 3E. Images of cells co-expressing H2B tagged with miRFP670nano3 and a-tubulin tagged with miRFP718nano. Scale bars. 10 μm. FIG. 3F. Dissociated rat cortical neurons transfected with miRFP670nano3 encoding plasmid. FIG. 3G. Dissociated rat cortical neurons transfected with miRFP704nano encoding plasmid. FIG. 3H. Dissociated rat cortical neurons transfected with miRFP718nano encoding plasmid. Scale bar. 10 μm.

FIGS. 4A, 4B, 4C, and 4D. Comparison of two-domain miRFPs and single-domain miRFPnanos structures and their chromophores. The BV chromophores in miRFP670nano3 (FIG. 4A), miRFP670) FIG. 4B), miRFP718nano FIG. 4C), and miRFP709 (FIG. 4D) bound to the respective Cys residues and their chemical formulas. Sticks representations show only rings A and B of the chromophores and Cys residues. In miRFP670nano3, the BV chromophore (FIG. 4A) is bound to Cys86 via the C3¹ atom. In miRFP670 (FIG. 4B), two chromophore-binding modes are observed. In this protein, the BV can be bound to either Cys253 in the GAF domain via its C3² atom (upper formula), or to both Cys253 in the GAF, and Cys20 in the PAS domain via its C3¹ and C3² atoms, respectively (lower formula), miRFP718nano (FIG. 4C) and miRFP709 (FIG. 4D) have the same chromophore species bound to the Cys57 and Cys20, respectively.

FIGS. 5A, 5B, 5C, and 5D. Design and evaluation of nanobodies (Nbs) internally fused with miRFP670nanos (NIR-Fbs) in live HeLa cells. FIG. 5A. Structure of Nb against GFP (Nb_{GFP}) (PDB ID: 3OGO) with indicated positions for insertion of miRFP670nano3. FIG. 5B. The amino acid sequence of nanobody to GFP (SEQ ID NO:8) with positions for insertion of miRFPnanos and corresponding linkers (GGGGS (SEQ ID NO:30). GGSGGGS (SEQ ID NO:31), or GGGGSGGGGS (SEQ ID NO:32)) indicated by arrows. The complementarity determining regions (CDRs) are in bold. FIG. 5C. The fluorescence intensity distribution of cells transfected with Nb_{GFP}, containing miRFP670nano3 inserted at G44/K45, S65/V66, or P90/E91 positions and co-expressed with or without EGFP cognate antigen analyzed by flow cytometry. FIG. 5D. Quantification of the data presented in FIG. 5C. Error bars, s.d. (n=3; transfection experiments).

FIG. 6. Design and evaluation of NIR-Fbs. The fluorescence intensity distribution of live Hela cells transiently transfected with Nb_{GFP} containing miRFP670nano3 inserted at G44/K45. S65/V66 or P90/E91 sites with corresponding linkers co-expressed with or without EGFP was analyzed using flow cytometry. Linker sequences: GGGGS (SEQ ID NO:30). GGSGGGS (SEQ ID NO:31). GGGGSGGGGS (SEQ ID NO:32). Error bars, s.d. (n=3; transfection experiments).

FIGS. 7A, 7B, 7C, 7D, and 7E. Analysis of antigen-binding properties of NIR-Fb_{GFP} using dot-blot assay. FIG. 7A. Live E, coli bacterial cells expressing mCherry (negative control) and NIR-Fb_{GFP}. Bacterial streaks were imaged with Leica M205FA fluorescence stereomicroscope using filter sets ex. 650/45 nm and em. 710/50 nm for NIR-Fb_{GFP} and ex. 560/40 nm and em. 620/40 nm for mCherry. FIG. 7B. Schematics of the dot-blot immunoassay for detection of EGFP immobilized on nitrocellulose membrane using NIR-Fb_{GFP}. FIG. 7C. Detection of immobilized EGFP using NIR-Fb_{GFP}. FIG. 7D. Scheme of the dot-blot immunoassay for detection of NIR-Fb_{GFP} immobilized on nitrocellulose membrane using EGFP. FIG. 7E. Detection of immobilized NIR-Fb_{GFP} using EGFP. Membranes were imaged with Leica M205FA fluorescence stereomicroscope using filter sets ex. 650/45 nm and em. 710/50 nm for NIR-Fb_{GFP} and ex. 480/40 nm and em. 535/50 nm for EGFP.

FIGS. 8A and 8B. Analysis of NIR-Fb specificity to an antigen. FIG. 8A. The fluorescence intensity distribution of live HeLa cells transiently transfected with NIR-Fb_{GFP} co-expressed with EGFP or with EGFP/N146I mutant was analyzed using flow cytometry. FIG. 8B. Quantification of the data presented in FIG. 8A.

FIGS. 9A, (B, and 9C. Evaluation of nanobodies (Nbs) internally fused with miRFP670nanos (NIR-Fbs) in live HeLa cells. FIG. 9A. Fluorescence intensity of cells transfected with the same amount of NIR-Fb_{GFP} plasmid and indicated amount of EGFP plasmid analyzed by flow cytometry. FIG. 9B. Mean fluorescence intensity of cells transfected with Nb_{GFP}, containing miRFP670nano3 inserted at G44/K45. S65/V66 or P90/E91 sites via Gly₄Ser linkers before and after 4 h of incubation with 10 μM bortezomib. FIG. 9C. Mean fluorescence intensity of HeLa cells transiently transfected with Nb_{GFP} containing miRFP670nano3 inserted at the indicated sites via corresponding linkers before and after 4 h of incubation with 10 μM bortezomib. Linker sequences: GGGGS (SEQ ID NO:30). GGSGGGS (SEQ ID NO:31). GGGGSGGGGS (SEQ ID NO:32). Error bars, s.d. (n=3; transfection experiments).

FIGS. 10A, 10B, 10C, and 10D. Re-coloring of EGFP fusions with Nb_{GFP} internally tagged with miRFP704nano (NIR₇₀₄-Fb_{GFP}) and miRFP718nano (NIR₇₁₈-Fb_{GFP}). FIG. 10A. Schematic representation of NIR₇₀₄-Fb_{GFP}. FIG. 10B. EGFP-a-tubulin fusion in live Hela cells re-colored into NIR with NIR₇₀₄-Fb_{GFP}. FIG. 10C. Schematic representation of NIR₇₁₈-Fb_{GFP}. FIG. 10D. EGFP-H2B fusion re-colored into NIR with NIR₇₁₈-Fb_{GFP}. miRFP704nano and miRFP718nano were inserted at the S65/V66 residues of Nb_{GFP} using the Gly₄Ser linker. Scale bars. 10 μm.

FIG. 11. Alignment of the amino acid sequences of the Nbs to GFP, mCherry, all actins, human β-catenin, ALFA-tag peptide, dihydrofolate reductase from E, coli, HIV's protein antigens p24 and gp41, and Nb21 and Nbm6 to SARS-COV-2's spike protein. The insertion position of miRFPnano3 is indicated by the arrow. Nanobody sequences can be found in Table 3.

FIGS. 12A and 12B. Evaluation of nanobodies (Nbs) internally fused with miRFP670nanos (NIR-Fbs) in live Hela cells. FIG. 12A. The fluorescence intensity distribution of cells transfected with NIR-Fbs to indicated antigens and co-expressed with or without cognate antigen was analyzed by flow cytometry. FIG. 12B. Fluorescence images of cells transfected with NIR-Fbs to indicated antigens and co-expressed with msfGFP-labeled cognate antigens or EGFP. Scale bars. 10 μm.

FIGS. 13A, 13B, 13C, and 13D. Re-coloring of EGFP and mCherry fluorescent proteins with NIR-Fbs: FIGS. 13A and 13B. Re-coloring of EGFP with NIR-Fb_{GFP}. FIG. 13A. EGFP-β-actin labeled with NIR-Fb_{GFP}. FIG. 13B. EGFP-α-tubulin labeled with NIR-Fb_{GFP}. Same order of images as in FIG. 13A. FIGS. 13C and 13D. Re-coloring of EGFP with NIR-Fb_{mCherry}. FIG. 13C, mCherry-B-actin labeled with NIR-Fb_{mCherry}. FIG. 13D, mCherry-α-tubulin labeled with NIR-Fb_{mCherry}. Same order of images as in FIG. 13C. Scale bars. 10 μm.

FIGS. 14A and 14B. Labeling of intracellular proteins with NIR-Fbs in live HeLa cells. FIG. 14A. Schematic representation of NIR-Fb_{ALFA} to ALFA tag. FIG. 14B. ALFA-tagged α-tubulin, B-actin, myosin, and clathrin labeled with NIR-Fb_{ALFA}. Scale bars, 10 μm.

FIGS. 15A and 15B. Labeling of intracellular proteins with NIR-Fbs in live HeLa cells. FIG. 15A. Left. Schematic representation of NIR-Fb_{actin} to β-actin. Right. Endogenous β-actin labeled with NIR-Fb_{actin}. FIG. 15B. Left. Schematic representation of non-fluorescent NIR-Fb_{actin}/Y58C fused with EGFP for indirect EGFP-labeling of β-actin. Right. Endogenous β-actin labeled with NIR-Fb_{actin}/Y58C-EGFP. Scale bars. 10 μm.

FIGS. 16A and 16B. NIR-Fb fusions with antigen-dependent properties. FIG. 16A. Schematic representation of the bispecific NIR-Fb_{GFP}-NIR-Fb_{mCherry} fusion. If at least one cognate antigen (EGFP or mCherry) is not expressed in the cell, the whole fusion degrades. FIG. 16B. Fluorescence images of live HeLa cells transiently co-transfected with (1) NIR-Fb_{GFP}-NIR-Fb_{mCherry}. EGFP cognate antigen and mCherry cognate antigen. (2) with NIR-Fb_{GFP}-NIR-Fb_{mCherry} and EGFP only, or (3) with NIR-Fb_{GFP}-NIR-Fb_{mCherry} and mCherry only. Scale bar. 10 μm.

FIGS. 17A, 17B, 17C, 17D, 17E, 17F, and 17G. Antigen-dependent stabilization of NIR-Fb fusion-partner. FIG. 17A. Schematic representation of NIR-Fb_{mCherry}-EGFP fusion. If mCherry is not expressed by cells, the fusion degrades. FIG. 17B. Fluorescent images of live HeLa cells transiently co-transfected with NIR-Fb_{mCherry}-EGFP and mCherry cognate antigen (top row), or with NIR-Fb_{mCherry}-EGFP and mTagBFP2 control (bottom row). FIG. 17C. Schematic representation of NIR-Fb_{GFP}-GAL4 and biNIR-Fb_{GFP}-GAL4 fusions for the antigen-dependent protein expression. In the presence of cognate antigen (EGFP). NIR-Fb_{GFP}-GAL4 or biNIR-Fb_{GFP}-GAL4 fusions drive expression of Gaussia luciferase (Gluc) reporter. Without cognate antigen. NIR-Fb_{GFP}-GAL4 and biNIR-Fb_{GFP}-GAL4 fusions degrade, resulting in no Gluc expression. Bioluminescence signal of live HeLa cells co-transfected with 5×UAS Gluc, either EGFP or mTagBFP2, and NIR-Fb_{GFP}-GAL4 (FIG. 17D) or biNIR-Fb_{GFP}-GAL4 (FIG. 17E). FIG. 17F. Schematic representation of NIR-Fb_{GFP}-GAL4 fusion for the antigen-dependent protein expression. In the presence of the cognate antigen (EGFP). NIR-Fb_{GFP}-GAL4 fusion drives the expression of the mCherry reporter. If the cognate antigen is not expressed the NIR-Fb_{GFP}-GAL4 fusion degrades, resulting in no mCherry expression. FIG. 17G. Fluorescence images of live HeLa cells transiently co-transfected with (1) NIR-Fb_{GFP}-GAL4, 5×UAS mCherry reporter, and EGFP or (2) NIR-Fb_{GFP}-GAL4, 5×UAS mCherry reporter, and mTagBFP2 control.

FIGS. 18A, 18B, 18C, 18D, and 18E. NIR-Fb fusion proteins for the targeted degradation of cellular proteins. FIG. 18A. Schematic representation of the NIR-Fb_{mCherry} fused with the additional Nb against a target antigen (in this case EGFP). In the absence of the mCherry cognate antigen, the whole fusion degrades together with the bound target antigen (EGFP). FIG. 18B. Fluorescence images of live HeLa cells transiently co-transfected with (1) NIR Fb_{mCherry}-Nb_{GFP}. EGFP and mCherry or (2) with NIR-Fb_{mCherry}-Nb_{GFP}. EGFP and mTagBFP2 control. FIG. 18C. Schematic representation of NIR-Fb_{GFP}-Nb_{ALFA} and biNIR-Fb_{GFP}-Nb_{ALFA} fusions for antigen-dependent degradation of ALFA-tagged proteins. In the presence of cognate antigen (EGFP). ALFA-tagged GAL4 drives expression of Gluc reporter, whereas without EGFP the NIR-FB fusions degrade together with bound ALFA-tagged GAL4. Bioluminescence signal of live HeLa cells co-transfected with ALFA-tagged GAL4, 5×UAS Gluc reporter, either EGFP or mTagBFP2, and (FIG. 18E) NIR-Fb_{GFP}-Nb_{ALFA} or (FIG. 18F) biNIR-Fb_{GFP}-Nb_{ALFA}.

FIGS. 19A, 19B, and 19C. NIR-Fb fusions for modulation of protein kinase activity. FIG. 19A. Schematic representation of non-fluorescent NIR-Fb_{GFP}/Y58C-PKI fusions with kinase inhibitory peptides (PKIs). In the presence of the cognate antigen (in this case EGFP), the fusions inhibit the kinases' activity. If the antigen is not expressed the fusions degrade. FIG. 19B. Live HeLa cells expressing NIR fluorescent PKA biosensor, transiently co-transfected with NIR-Fb_{GFP}/Y58C-PKI and EGFP cognate antigen or with NIR-Fb_{GFP}/Y58C-PKI and mTagBFP2 control. Cells were imaged before and 45 min after the stimulation with dbcAMP. Control is cells not transfected with NIR-Fb_{GFP}/Y58C-PKI. FIG. 19C. Live HeLa cells expressing NIR fluorescent JNK biosensor transiently transfected with NIR-Fb_{GFP}/Y58C-JIP and EGFP cognate antigen, or with NIR-Fb_{GFP}/Y58C-JIP and mTagBFP2 control. Cells were imaged before and 45 min after the stimulation with 1 μg/ml anisomycin. FRET/donor ratio images are presented using intensity pseudocolor. Control is cells not transfected with NIR-Fb_{GFP}/Y58C-JIP. Error bars, s.d. (n=3; transfection experiments). At least 15 cells were analyzed in each experiment.

FIGS. 20A and 20B. NIR-Fbs-based intracellular immunotoxin. FIG. 20A. HEK-293T cells were co-transfected with biNIR-Fb_{GFP}-caspase/V266E fusion and with EGFP or mTagBFP2, respectively. Annexin V-positive cells were quantified 24 h after transfection. FIG. 20B. HeLa cells were co-transfected with biNIR-Fb_{GFP}-DTA fusion and with EGFP or mTagBFP2, respectively. Cell viability was quantified by MTT test 48 h after transfection. Error bars, s.d. (n=3; transfection experiments).

FIGS. 21A and 21B. Analysis of NIR-Fbs secretion in the presence or w/o specific antigen. FIG. 21A. Bioluminescence signal of live HeLa cells co-transfected with Gluc-biNIR-Fb_{GFP} and either EGFP or mTagBFP2 24 h, 48 h and 72 h after transfection. FIG. 21B. Ratio of bioluminescent signals of HeLa cells co-transfected with Gluc-biNIR-Fb_{GFP} and either EGFP or mTagBFP2 24 h, 48 h and 72 h after transfection. Error bars, s.d. (n=3; transfection experiments).

### DETAILED DESCRIPTION

Provided herein are fusion proteins comprising a single domain antibody (sdAb) (including, but not limited to, a nanobody) that binds selectively to a specific antigen, wherein a second polypeptide is inserted into the single domain antibody, generating an internal fusion. Also provided are methods of making and methods of using the fusion proteins disclosed herein.

As a non-limiting example, the fusion proteins disclosed herein may be used for imaging. Fusion proteins disclosed herein enable re-coloring of cellular structures labeled with visible range fluorescent proteins (FPs), e.g., EGFP or mCherry, using fusion proteins comprising Nbs against these FPs.

Additionally, NIR-Fb fusion proteins comprising Nbs against intracellular proteins are capable of efficiently binding and visualizing endogenous proteins, and background-free images are observed.

Moreover, fusion proteins disclosed herein enable manipulation of endogenous molecules by serving as a destabilizing fusion-partner for various effector proteins and peptides. To this end, molecular constructs were developed for directed degradation of targeted proteins, controllable protein expression, and modulation of the activity of enzymes in an antigen-dependent manner.

Additionally, fusion proteins disclosed herein can be used for the detection of rare endogenous proteins (i.e., can be used as biosensors) and for the manipulation of various endogenous proteins, signaling pathways, and even cell fate. For example, NIR-Fbs specific to active conformations of signaling molecules can enable monitoring their activity and depending on intracellular localization. Moreover, fusions of fusion proteins disclosed herein with Cre, Cas9 and Flp recombinases or transcriptional factors can control gene expression and genome editing selectively in cells, expressing specific intracellular epitopes.

Notably, because NIR-Fbs exhibit bright fluorescence in near-infrared tissue transparency window, they can be used in living mammals, including humans, in various deep-tissue applications. For example, NIR-Fbs specific for cancer biomarker or pathogen (viruses or bacteria) antigens, fused with cellular toxin and delivered by AAV vehicle, can enable initially visualization and then elimination of the tumor or pathogen-infected cells in a subject.

Due to the high homology and similar structural organization among different NBs, it has been discovered that the Nb fusion approach described herein is generalizable and works well for a large variety of Nbs. Accordingly, the fusion proteins disclosed herein open completely new ways for development and screening for high-affinity Nbs and their uses in cells and in vivo. Nbs can be screened as a library of NIR-Fb fusions expressed in mammalian cells using flow cell sorters, thus enormously speeding up screening, allowing screening of significantly larger Nb synthetic and naïve libraries, and yielding Nbs with higher affinity that are well-folded in mammalian cells, as compared to current phage, cell-surface and ribosome displays, which mainly allow enriching Nb repertoires.

Fluorescent Fusion Proteins that Specifically Bind to Targets of Interest

Provided herein are fusion proteins comprising a single domain antibody (sdAb) that binds selectively to a specific antigen, wherein a second polypeptide is inserted into the single domain antibody, generating an internal fusion.

As used herein, the term “single domain antibody” or “sdAb” refers to an antibody fragment consisting of a single monomeric variable antibody domain. In one embodiment, the sdAb is derived from the antigen-binding portion of a camelid heavy-chain-only antibody. Such an sdAb is also referred to as a VHH fragment or a nanobody. In one embodiment, the sdAb is derived from the antigen-binding portion of a cartilaginous fish heavy-chain-only antibodies (IgNAR, ‘immunoglobulin new antigen receptor’). Such an sdAb is called a VNAR fragment. Alternatively, an sdAb can be generated from conventional IgGs by obtaining or engineering monomeric, stable VH or VL domains.

A single-domain antibody comprises a variable region primarily responsible for antigen recognition and binding and a framework region. The “variable region,” also called the “complementarity determining region” (CDR), comprises loops which differ extensively in size and sequence based on antigen recognition. CDRs are generally responsible for the binding specificity of the single-domain antibody. Distinct from the CDRs is the framework region. The framework region is relatively conserved and assists in overall protein structure. The framework region may comprise a large solvent-exposed surface consisting of a ß-sheet and loop structure.

The sdAbs disclosed herein can be directed against any antigen, including, but not limited to, a fluorescent protein, a tumor antigen, or an intracellular antigen.

Examples of fluorescent proteins that the sdAb may bind to include, but are not limited to, a red fluorescent protein, green fluorescent protein, (3-F)Tyr-EGFP, A44-KR, aacuGFP1, aacuGFP2, aceGFP, aceGFP-G222E-Y220L, aceGFP-h, AcGFP1, AdRed, AdRed-C148S, aeurGFP, afraGFP, alajGFP1, alajGFP2, alajGFP3, amCyan1, amFP486, amFP495, amFP506, amFP515, amilFP484, amilFP490, amilFP497, amilFP504, amilFP512, amilFP513, amilFP593, amilFP597, anm1GFP1, anm1GFP2, anm2CP, anobCFP1, anobCFP2, anobGFP, apulFP483, AQ14, AQ143, Aquamarine, asCP562, asFP499, AsRed2, asulCP, atenFP, avGFP, avGFP454, avGFP480, avGFP509, avGFP510, avGFP514, avGFP523, AzamiGreen, Azurite, BDFP1.6, bfloGFPal, bfloGFPcl, BFP. BFP.A5, BFP5, bsDronpa (On), ccalGFP1, ccalGFP3, ccalOFP1, ccalRFP1, ccalYFP1, cEGFP, cerFP505, Cerulean, CFP, cFP484, cfSGFP2, cgfmKate2, CGFP, cgfTagRFP, cgigGFP, cgreGFP, CheGFP1, CheGFP2, CheGFP4, Citrine, Citrine2, Clomeleon, Clover, cp-mKate, cpCitrine, cpT-Sapphire174-173, CyOFP1, CyPet, CyRFP1 (CyRFP1), d-RFP618, D10, d1EosFP (Green), d1EosFP (Red), d2EosFP (Green), d2EosFP (Red), deGFP1, deGFP2, deGFP3, deGFP4, dendFP (Green), dendFP (Red), Dendra (Green), Dendra (Red), Dendra2 (Green), Dendra2 (Red), Dendra2-M159A (Green), Dendra2-M159A (Orange), Dendra2-T69A (Green), Dendra2-T69A (Orange), dfGFP, dimer1, dimer2, dis2RFP, dis3GFP, dKeima, dKeima570, dLanYFP, DrCBD, Dreiklang (On), Dronpa (On), Dronpa-2 (On), Dronpa-3 (On), dsFP483, DspR1, DsRed, DsRed-Express, DsRed-Express2, DsRed-Max, DsRed.M1, DsRed.T3, DsRed, T4, DsRed2, DstC1, dTFP0.1, dTFP0.2, dTG, dTomato, dVFP, E2-Crimson, E2-Orange, E2-Red/Green, EaGFP, EBFP, EBFP1.2, EBFP1.5, EBFP2, ECFP, ECFPH148D, ECGFP, eechGFP1, eechGFP2, eechGFP3, eechRFP, efasCFP, efasGFP, eforCP, EGFP, eGFP203C, eGFP205C, Emerald, Enhanced Cyan-Emitting GFP, EosFP (Green), EosFP (Red), eqFP578, eqFP611, eqFP611V124T, eqFP650, eqFP670, EYFP, EYFP-Q69K, fabdGFP, ffDronpa (On), FoldingReporterGFP, FP586, FPrf12.3, FR-1, FusionRed, FusionRed-M, G1, G2, G3, Gamillus (On), Gamillus0.1, Gamillus0.2, Gamillus0.3, Gamillus0.4, GCaMP2, gfasGFP, GFP(S65T), GFP-151pyTyrCu, GFP-Tyr151pyz, GFPmut2, GFPmut3, GFPxm16, GFPxm161, GFPxm162, GFPxm163, GFPxm18, GFPxm181uv, GFPxm18uv, GFPxm19, GFPxm191uv, GFPxm19uv, H9, HcRed, HcRed-Tandem, HcRed7, hcriGFP, hmGFP, HriCFP, HriGFP, iFP1.4, iFP2.0, iLov, iq-EBFP2, iq-mApple, iq-mCerulean3, iq-mEmerald, iq-mKate2, iq-mVenus, iRFP670, iRFP682, iRFP702, iRFP713, iRFP720, IrisFP (Green), IrisFP (Orange), IrisFP-M159A (Green), Jred, Kaede (Green), Kaede (Red), Katushka, Katushka-9-5, Katushka2S, KCY, KCY-G4219, KCY-G4219-38L, KCY-R1, KCY-R1-158A, KCY-R1-38H, KCY-R1-38L, KFP1 (On), KikGR1 (Green), KikGR1 (Red), KillerOrange, KillerRed, KO, Kohinoor (On), laesGFP, laGFP, LanFP1, LanFP2, lanRFP-ΔS831, LanYFP, laRFP, LSS-mKate1, LSS-mKate2, LSSmOrange, M355NA, mAmetrine, mApple, Maroon0.1, mAzamiGreen, mBanana, mBeRFP, mBlueberry 1, mBlueberry2, mc1, mc2, mc3, mc4, mc5, mc6, McaG1, McaG1ea, McaG2, mCardinal, mCarmine, mcavFP, mcavGFP, mcavRFP, mcCFP, mCerulean, mCerulean.B, mCerulean.B2, mCerulean.B24, mCerulean2, mCerulean2.D3, mCerulean2.N, mCerulean2.N(T65S), mCerulean3, mCherry, mCherry2, mCitrine, mClavGR2 (Green), mClavGR2 (Red), mClover3, mCyRFP1, mECFP, meffCFP, meffGFP, meffRFP, mEGFP, meleCFP, meleRFP, mEmerald, mEos2 (Green), mEos2 (Red), mEos2-A69T (Green), mEos2-A69T (Orange), mEos3.1 (Green), mEos3.1 (Red), mEos3.2 (Green), mEos3.2 (Red), mEos4a (Green), mEos4a (Red), mEos4b (Green), mEos4b (Red), mEosFP (Green), mEosFP (Red), mEosFP-F173S (Green), mEosFP-F173S (Red), mEosFP-M159A (Green), mEYFP, MfaG1, mGarnet, mGarnet2, mGeos-C(On), mGeos-E (On), mGeos-F (On), mGeos-L (On), mGeos-M (On), mGeos-S(On), mGinger1, mGinger2, mGrape1, mGrape2, mGrape3, mHoneydew, MiCy, mIFP, miniSOG, miniSOGQ103V, miniSOG2, miRFP, miRFP670, miRFP670nano, miRFP670v1, miRFP703, miRFP709, miRFP720, mIrisFP (Green), mIrisFP (Red), mK-GO (Early), mK-GO (Late), mKalama1, mKate, mKateM41GS158C, mKateS158A, mKateS158C, mKate2, mKeima, mKelly1, mKelly2, mKG, mKikGR (Green), mKikGR (Red), mKillerOrange, mKO, mKO2, mKOK, mLumin, mMaple (Green), mMaple (Red), mMaple2 (Green), mMaple2 (Red), mMaple3 (Green), mMaple3 (Red), mMaroon1, mmGFP, mMiCy, mmilCFP, mNectarine, mNeonGreen, mNeptune, mNeptune2, mNeptune2.5, mNeptune681, mNeptune684, Montiporasp, #20-9115, mOrange, mOrange2, moxBFP, moxCerulean3, moxDendra2 (Green), moxDendra2 (Red), moxGFP, moxMaple3 (Green), moxMaple3 (Red), mox NeonGreen, mox Venus, mPapaya, mPapaya0.7, mPlum, mPlum-E16P, mRaspberry, mRed7, mRed7Q1, mRed7Q1S1, mRed7Q1S1BM, mRFP1, mRFP1-Q66C, mRFP1-Q66S, mRFP1-Q66T, mRFP1.1, mRFP1.2, mRojoA, mRojoB, mRouge, mRtms5, mRuby, mRuby2, mRuby 3, mScarlet, mScarlet-H, mScarlet-I, mStable, mStrawberry, mT-Sapphire, mTagBFP2, mTangerine, mTFP0.3, mTFP0.7 (On), mTFP1, mTFP1-Y67W, mTurquoise, mTurquoise2, muGFP, mUkG, mVenus, mVenus-Q69M, mVFP, mVFP1, mWasabi, Neptune, NijiFP (Green), NijiFP (Orange), NowGFP, obeCFP, obeGFP, obeYFP, OFP, OFPxm, oxBFP, oxCerulean, oxGFP, ox Venus, P11, P4, P4-1, P4-3E, P9, PA-GFP (On), Padron (On), Padron(star) (On). Padron0.9 (On), PAmCherry1 (On), PAmCherry2 (On), PAmCherry3 (On), PAmKate (On), PATagRFP (On), PATagRFP1297 (On), PATagRFP1314 (On), pcDronpa (Green), pcDronpa (Red), pcDronpa2 (Green), pcDronpa2 (Red), PdaC1, pdae1GFP, phiYFP, phiYFPv, pHluorin, ecliptic, pHluorin, ecliptic (acidic), pHluorin, ratiometric (acidic), pHluorin, ratiometric (alkaline), pHluorin2 (acidic), pHluorin2 (alkaline), pHuji, PlamGFP, pmeaGFP1, pmeaGFP2, pmimGFP1, pmimGFP2, Pp2FbFP, Pp2FbFPL30M, ppluGFP1, ppluGFP2, pporGFP, pporRFP, PS-CFP (Cyan), PS-CFP (Green), PS-CFP2 (Cyan), PS-CFP2 (Green), psamCFP, PSmOrange (Far-red), PSmOrange (Orange), PSmOrange2 (Far-red), PSmOrange2 (Orange), ptilGFP, R3-2+PCB, RCaMP, RDSmCherry0.1, RDSmCherry0.2, RDSmCherry0.5, RDSmCherry 1, rfloGFP, rfloRFP, RFP611, RFP618, RFP630, RFP637, RFP639, roGFP1, roGFP1-R1, roGFP1-R8, roGFP2, rrenGFP, RRYT, rsCherry (On), rsCherry Rev (On), rsCherryRev1.4 (On), rsEGFP (On), rsEGFP2 (On), rsFastLime (On), rsFolder (Green), rsFolder2 (Green), rsFusionRed1 (On), rsFusionRed2 (On), rsFusionRed3 (On), rsTagRFP (ON), Sandercyanin, Sapphire, sarcGFP, SBFP1, SBFP2, SCFP1, SCFP2, SCFP3A, SCFP3B, scubGFP1, scubGFP2, scubRFP, secBFP2, SEYFP, sg11, sg12, sg25, sg42, sg50, SGFP1, SGFP2, SGFP2(206A), SGFP2(E222Q). SGFP2(T65G), SHardonnay, shBFP, shBFP-N158S/L173I, ShG24, Sirius, SiriusGFP. Skylan-NS (On), Skylan-S(On), smURFP, SNIFP, SOPP, SOPP2, SOPP3, SPOON (on), stylGFP, SuperfolderGFP, SuperfoldermTurquoise2, SuperfoldermTurquoise2ox, SuperNovaGreen, SuperNovaRed, SYFP2, T-Sapphire, TagBFP, TagCFP, TagGFP, TagGFP2, TagRFP, TagRFP-T, TagRFP657, TagRFP675, TagYFP, td-RFP611, td-RFP639, tdimer2(12), tdKatushka2, TDsmURFP, tdTomato, tKeima, Topaz, TurboGFP, TurboGFP-V197L, TurboRFP, Turquoise-GL, Ultramarine, UnaG, usGFP, Venus, VFP, vsfGFP-0, vsfGFP-9, W1C, W2, W7, WasCFP, Wi-Phy, YPet, zFP538, zoan2RFP, ZsGreen, ZsYellow1, αGFP, 10B, 22G, 5B, 6C, Ala, aacuCP, acanFP, ahyaCP, amilCP, amilCP580, amilCP586, amilCP604, apulCP584, BFPsol, Blue102, CFP4, cgigCP, CheGFP3, Clover1.5, cpasCP, Cy11.5, dClavGR1.6, dClover2, dClover2A206K, dhorGFP, dhorRFP, dPapaya0.1, Dronpa-C62S, DsRed-Timer, echFP, echiFP, EYFP-F46L, fcFP, fcomFP, Fpaagar, Fpag_frag, Fpcondchrom, FPmann, FPmcavgr7.7, Gamillus0.5, gdjiCP, gfasCP, GFPhal, gtenCP, hcriCP, hfriFP, KikG, LEA, mcFP497, mcFP503, mcFP506, mCherry 1.5, mClavGR1, mClavGR1.1, mClavGR1.8, mClover1.5, mcRFP, meffCP, mEos2-NA, meruFP, mKate2.5, mOFP.T.12, mOFP.T.8, montFP, moxEos3.2, mPA-GFP, mPapaya0.3, mPapaya0.6, mRFP1.3, mRFP1.4, mRFP1.5, mTFP0.4, mTFP0.5, mTFP0.6, mTFP0.8, mTFP0.9, mTFP1-Y67H, mTurquoise-146G, mTurquoise-146S, mTurquoise-DR, mTurquoise-GL, mTurquoise-GV, mTurquoise-RA, mTurquoise2-G, NpR3784g, PDM1-4, psupFP, Q80R, rfloGFP2, RpBphP1, RpBphP2, RpBphP6, rrGFP, RSGFP1, RSGFP2, RSGFP3, RSGFP4, RSGFP6, RSGFP7, Rtms5, scleFP1, scleFP2, spisCP, stylCP, sympFP, TeAPCα, tPapaya0.01, Trp-lessGFP, vsGFP, Xpa, yEGFP, YFP3, zGFP, and zRFP.

The term “tumor antigen” as used herein includes both tumor associated antigens (TAAs) and tumor specific antigens (TSAs). A tumor associated antigen means an antigen that is expressed by a tumor cell in higher amounts than is expressed by normal cells or an antigen that is expressed by normal cells during fetal development. A tumor specific antigen is an antigen that is unique to tumor cells and is not expressed by normal cells. The term tumor antigen includes TAAs or TSAs that have been already identified and those that have yet to be identified and includes fragments, epitopes and any and all modifications to the tumor antigens. Not-limiting examples of tumor antigens that the sdAbs may bind to include, but are not limited to, CD19, CD20, CD30, CD33, CD38, CD133, BCMA, TEM8, EpCAM, ROR1, Folate Receptor, CD70, MAGE-1, MAGE-2, MAGE-3, CEA, tyrosinase, midkin, BAGE, CASP-8, β-catenin, CA-125, CDK-1, ESO-1, gp75, gp100, MART-1, MUC-1, MUM-1, p53, PAP, PSA, PSMA, ras, trp-1, HER-2, TRP-1, TRP-2, IL 13Ralpha, IL13Ralpha2, AIM-2, AIM-3, NY-ESO-1, C9orf112, SART1, SART2, SART3, BRAP, RTN4, GLEA2, TNKS2, KIAA0376, ING4, HSPH1, C13orf24, RBPSUH, C6orf153, NKTR, NSEP1, U2AFIL, CYNL2, TPR GOLGA, BMII, COX-2, EGFRvIII, EZH2, LICAM, Livin, Livinβ, MRP-3, Nestin, OLIG2, ART1, ART4, B-cycline, Gli1, Cav-1, Cathepsin B, CD74, E-Cadherin, EphA2/Eck, Fra-1/Fosl 1, GAGE-1, Ganglioside/GD2, GnT-V, β1, 6-N, Ki67, Ku70/80, PROX1, PSCA, SOX10, SOX11, Survivin, βhCG, WT1, mesothelin, melan-A, NY-BR-1, NY-CO-58, MN (gp250)), telomerase, SSX-2, PRAME, PLK1, VEGF-A, VEGFR2, and Tie-2.

Intracellular proteins that the sdAb may bind to include, but are not limited to, antigens that are found, for example, in the cytoplasm and/or nucleus of a cell. Examples of an intracellular antigen include, but are not limited to, a receptor (e.g., cytoplasmic receptors such as peroxisome proliferator-activated receptors and nuclear receptors such as steroid hormone receptor, aryl hydrocarbon receptor), a transcription factor (e.g., SPI, AP-1, C/EBP, Heat shock factor, ATF/CREB, c-Myc, 1-Oct, NF-1, STAT3), a cytokine (e.g., interleukins, interferons, erythropoietin, thrombopoietin, colony stimulating factors), a growth factor (EGF, HGF, BMP, VEGF), an enzyme (e.g., protease, kinase, phosphatase), messengers (e.g., hormones such as vasopressin, follicle stimulating hormone, luteinizing hormone or neurotransmitters such as somatostatin or substance P), a member of a signaling pathway (e.g., MAPK pathway, Wnt pathway, Hedgehog pathway, Retinoic acid pathway, TGF beta pathway, JAK-STAT pathway, cAMP-dependent pathway), a carrier protein (e.g., electron carriers, such as oxidoreductases, NADPH oxidases), or a structural protein (e.g., actin, tubulin).

In one aspect, provided is an internal fusion protein comprising a nanobody, wherein a polypeptide is inserted into the nanobody.

In some embodiments, the inserted polypeptide is a fluorescent protein, a drug, a toxin, an enzyme, and/or a polypeptide with inhibitory function. In some embodiments, the polypeptide is a non-fluorescent protein. A non-limiting example of a non-fluorescent protein is the miRFP670nano3 variant Tyr58Cys (SEQ ID NO:7).

Non limiting examples of fluorescent proteins that may be inserted into the sdAbs include, but are not limited to, any of the fluorescent proteins disclosed herein. In some embodiments, the inserted protein is a near-infrared (NIR) fluorescent protein (FP). In one embodiment, provided is an internal fusion protein comprising a nanobody, wherein a polypeptide is inserted into the nanobody and wherein the polypeptide inserted into the nanobody is a miRFPnano. The resulting fusion protein is referred to as a near-infrared fluorescent nanobody (NIR-Fb). In some embodiments, the inserted protein is a miRPFnano variant. In some embodiments, the inserted protein comprises miRFP670nano, miRFP670nano R57C C86, miRFP670nano R57C C86S L114F V115S, miRFP670nano3, miRFP704nano, miRFP718nano, or a non-fluorescent miRFP670nano variant. In some embodiments, the miRFPnano is covalently linked to a chromophore. In some embodiments, the chromophore is biliverdin IVα (BV). In some embodiments, the chromophore is phycocyanobilin (PCB) tetrapyrrole.

Non limiting examples of drugs that may be inserted into the sdAbs include, but are not limited to, human growth hormone, growth hormone releasing hormone, growth hormone releasing peptide, interferons, colony stimulating factors, interleukins, macrophage activating factor, macrophage peptide, B cell factor, T cell factor, protein A, allergy inhibitor, cell necrosis glycoproteins, immunotoxin, lymphotoxin, tumor necrosis factor, tumor suppressors, metastasis growth factor, alpha-1 antitrypsin, albumin and fragment polypeptides thereof, apolipoprotein-E, erythropoietin, factor VII, factor VIII, factor IX, plasminogen activating factor, urokinase, streptokinase, protein C, C-reactive protein, renin inhibitor, collagenase inhibitor, superoxide dismutase, platelet-derived growth factor, epidermal growth factor, osteogenic growth factor, bone stimulating protein, calcitonin, insulin, atriopeptin, cartilage inducing factor, connective tissue activating factor, follicle stimulating hormone, luteinizing hormone, luteinizing hormone releasing hormone, nerve growth factors, parathyroid hormone, relaxin, secretin, somatomedin, insulin-like growth factor, adrenocortical hormone, glucagon, cholecystokinin, pancreatic polypeptide, gastrin releasing peptide, corticotropin releasing factor, thyroid stimulating hormone, monoclonal or polyclonal antibodies against various viruses, bacteria, toxins, etc., and virus-derived vaccine antigens, human serum albumin, human growth hormone, interferon alpha, erythropoietin, colony stimulating factors, immunoglobulins, angiogenin, bone morphogenic protein, chemokines, leptin, inhibitory factor, stem cell factor, transforming growth factor, and tumor necrosis factor.

Non limiting examples of toxins that may be inserted into the sdAbs include, but are not limited to, cholesterol dependent cytolysins, ADP-ribosylating toxins, plant toxins, bacterial toxins, viral toxins, pore forming toxins, and cell penetrating peptides. Non-limiting examples of toxins include caspase-3 mutant V266E, Pseudomonas exotoxin, ricin, gelonin, cucurmosin, diphtheria toxin fragment A, diphtheria toxin fragment A/B, tetanus toxin, E. coli heat labile toxin (LTI and/or LTII), cholera toxin, C. perfringes iota toxin, shiga toxin, anthrax toxin, MTX (B. sphaericus mosquilicidal toxin), perfringolysin 0, streptolysin, barley toxin, mellitin, anthrax toxins LF and EF, adenylate cyclase toxin, botulinolysin B, botulinolysin E3, botulinolysin C, botulinum toxin A, cholera toxin, clostridium toxins A, B, and alpha, shiga A toxin, shiga-like A toxin, cholera A toxin, pertussis Si toxin, E, coli heat labile toxin (LTB), pH stable variants of listeriolysin 0 (pH-independent; amino acid substitution L461T), thermostable variants of listeriolysin 0 (amino acid substitutions E247M, D320K), pH and thermostable variants of listeriolysin 0 (amino acid substitutions E247M, D320K, and L46IT), streptolysin 0), streptolysin c, streptolysin 0 e, sphaericolysin, anthrolysin 0, cereolysin, thuringiensilysin 0, 41 3275813vl weihenstephanensilysin, alveolysin, brevilysin, butyriculysin, tetanolysin 0, novyilysin, lectinolysin, pneumolysin, mitilysin, pseudopneumolysin, suilysin, intermedilysin, ivanolysin, seeligeriolysin 0, vaginolysin, and pyolysin.

Non limiting examples of enzymes that may be inserted into the sdAbs include, but are not limited to, lysozyme, pronase, serrapeptase, streptokinase, streptodolase, urokinase, hyaluronidase, beta-glucosidase, alpha-galactosidase, beta-galactosidase, iduronidase, and iduronate 2-Sulfatase (iduronate-2-sulfatase), galactose-6-sulfatase, alpha-glucosidase, acid ceramidase, acid sphingomyelina Acid sphingomyelinsase, galactocerebrosidsase, arylsulfatase A, B, beta-hexosaminidase A, B, heparin-N-sulfatase-sulfatase), alpha-D-mannosidase, beta-glucuronidase, N-acetylgalactosamine-6-sulfatase (N-acetylgalactosamine-6 sulfatase), Lysosomal acid lipase, alpha-N-acetyl-glucosaminidase, glucocere Brocidase (glucocerebrosidase), butyrylcholinesterase, chitinase, glutamate decarboxylase, imiglucerase, lipase, uricase, Platelet activating factor Acetylhydrolase, neutral endopeptidase, myeloperoxidase, iduronate sulfatase, Agalsidase, Taliglucerase, Velaglucerase, Alglucerase, Sebelipase, Laronidase, Idursulfase, Galsulfase, and Elosulfase.

In some embodiments, a polypeptide with inhibitory function that may be inserted into the sdAbs include, wherein the polypeptide reduces the biological activity of another biological molecule. In some embodiments, the polypeptide with inhibitory function is an enzyme inhibitor. In some embodiments, the polypeptide with inhibitory function is an antibody. In some embodiments, the polypeptide with inhibitory function binds to a regulatory sequence element and inhibits or activates the transcription of a sequence operatively linked to a regulatory sequence element, respectively.

In some embodiments, the polypeptide inserted into the nanobody is connected to the N-terminal and the C-terminal portion of the nanobody with a flexible linker. In some embodiments, the flexible linker is between 3 and 30 amino acids long. In some embodiments, the flexible linker predominantly comprises glycine and serine residues. In some embodiments, the flexible linker comprises a sequence selected from the group consisting of GGS, GGGGS (SEQ ID NO:30), GGSGGGS (SEQ ID NO:31), or GGGGSGGGGS (SEQ ID NO:32) or repeats of these sequences. In some embodiments, the polypeptide inserted into the nanobody is connected to the N-terminal and the C-terminal portion of the nanobody with rigid linkers.

In some embodiments, the fusion proteins disclosed herein comprise the following sequences from N- to C-terminus: N-terminal portion of the nanobody-linker 1-first polypeptide forming an alpha helix-inserted polypeptide-second polypeptide forming an alpha helix-linker 2-C-terminal portion of the nanobody.

In some embodiments, the fusion proteins disclosed herein comprise the following sequences from N- to C-terminus: N-terminal portion of the nanobody-linker 1-first polypeptide forming an alpha helix-linker 2-inserted polypeptide-linker 3-second polypeptide forming an alpha helix-linker 4-C-terminal portion of the nanobody.

The linkers may or may not have the same sequence/length. In some embodiments, one of the two polypeptides forming an alpha helix comprises the sequence MANLDKMLNTTVTEVRKF (SEQ ID NO:33) or a sequence that is at least 80%, at least 85%, at least 90%, at least 91%, at least 92%, at least 93%, at least 94%, at least 95%, at least 96%, at least 97%, at least 98%, or at least 99% identical thereto.

In some embodiments, one of the two polypeptides forming an alpha helix comprises the sequence TWEIDFLKQQAVVMGIAIQQS (SEQ ID NO:34) or a sequence that is at least 80%, at least 85%, at least 90%, at least 91%, at least 92%, at least 93%, at least 94%, at least 95%, at least 96%, at least 97%, at least 98%, or at least 99% identical thereto.

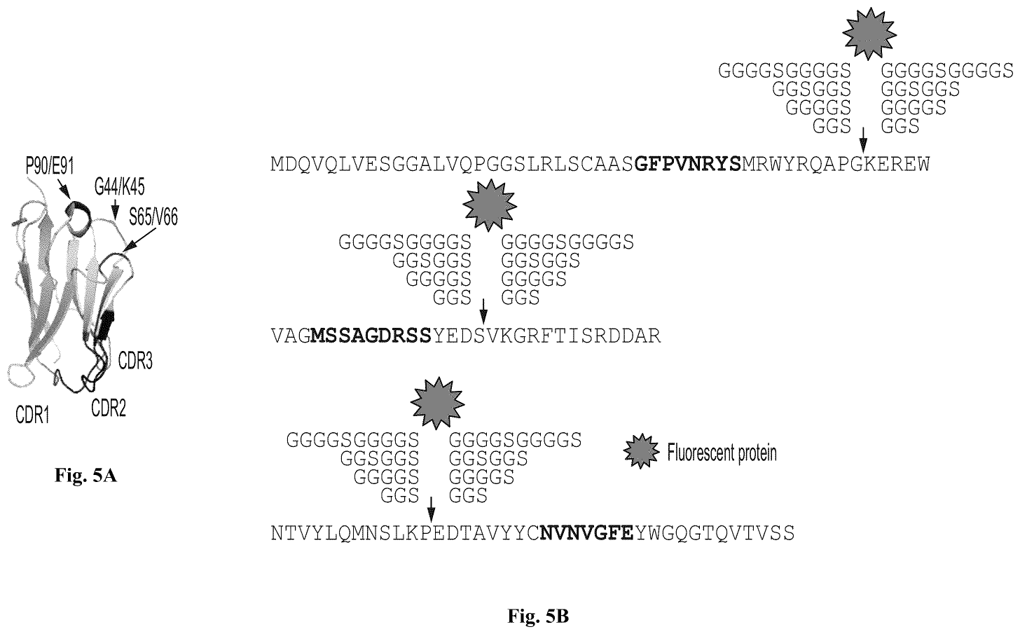

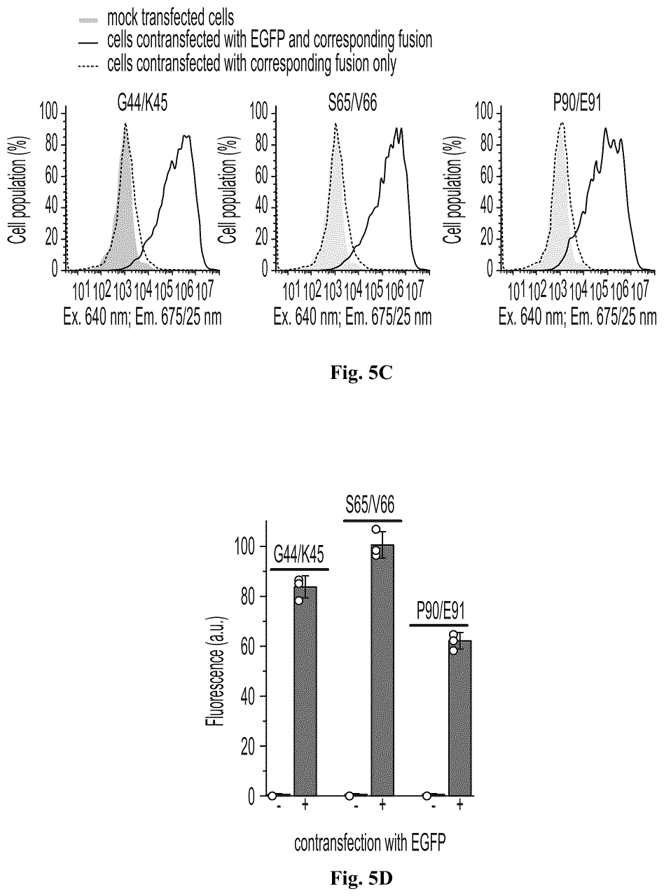

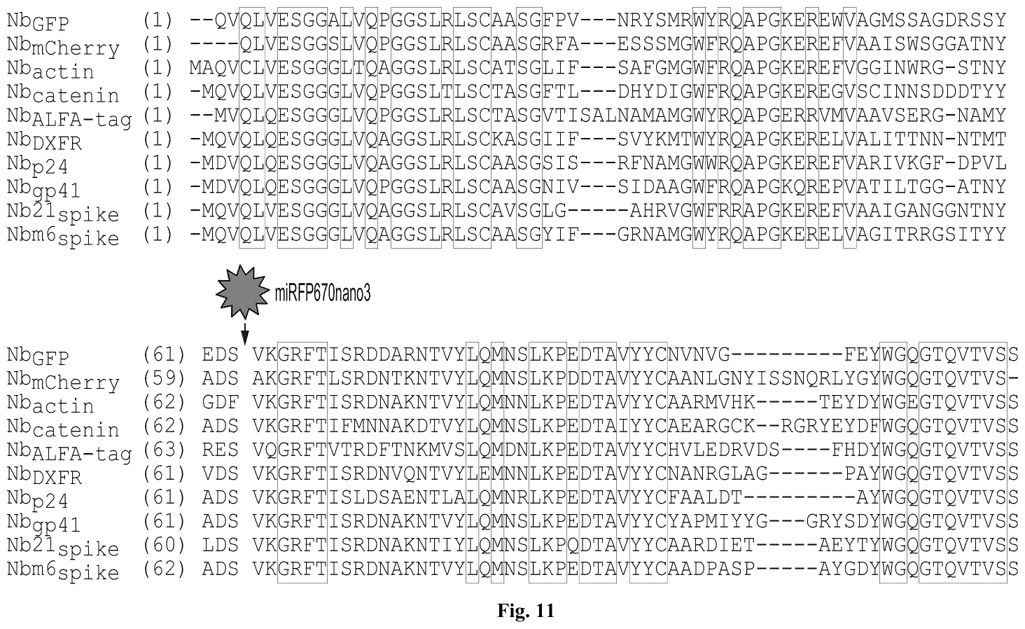

In some embodiments, the insertion site in the nanobody for the polypeptide (and any linkers and/or alpha helices forming sequences if applicable) corresponds to the G44/K45 site of the nanobody to GFP shown in FIG. 5. In some embodiments, the insertion site in the nanobody for the polypeptide (and any linkers and/or alpha helices forming sequences if applicable) corresponds to the S65/V66 site of the nanobody to GFP (Nb_{GFP}) shown in Fig. In some embodiments, the insertion site in the nanobody for the polypeptide (and any linkers and/or alpha helices forming sequences if applicable) corresponds to the P90/E91 site of the nanobody to GFP (Nb_{GFP}) shown in FIG. 5. Binding sites in other nanobodies corresponding to these insertion sites in the nanobody to GFP can be easily identified by a person skilled in the art, using, for example, an alignment. See, for example, FIG. 11.

In some embodiments, provided herein is a multi-modular fusion protein comprising an internal fusion protein disclosed herein fused to one more internal fusion proteins disclosed herein.

In some embodiments, the multi-modular fusion protein comprises two fusion proteins disclosed herein. In some embodiments, the nanobody of the first fusion protein binds to a different antigen than the nanobody of the second fusion protein, generating a bispecific multi-modular fusion protein. In some embodiments, the nanobody of the first fusion protein binds to the same antigen as the nanobody of the second fusion protein.

In some embodiments, the multi-modular fusion protein is fused to one or more additional proteins.

In embodiments, the multi-modular fusion protein a trispecific multi-modular fusion protein.

In some embodiments, the internal fusion proteins or the multi-modular fusion proteins disclosed herein comprise a signal sequence (also referred to as a signal peptide). Signal sequences are well known in the art. See, e.g., Owji et al., comprehensive review of signal peptides: Structure, roles, and applications. Eur J Cell Biol 2018 August; 97(6): 422-441, incorporated herein in its entirety. In one embodiment, the signal sequence comprises (MGVKVLFALICIAVAE, SEQ ID NO:35).

In some embodiments, provided is an internal fusion protein comprising a first nanobody directed against a first antigen, optionally wherein a fluorescent protein is inserted into the first nanobody, wherein the internal fusion protein is fused to second nanobody directed against a second antigen that is to be targeted for degradation.

In some embodiments, provided is an internal fusion protein comprising a nanobody directed against a first protein, wherein the internal fusion protein is fused to a transcription factor that controls expression of a second protein. In some embodiments, the transcription factor is GAL, which is used in the GAL4/UAS expression system.

In some embodiments, provided is an internal fusion protein comprising a nanobody directed against a protein of interest, wherein the internal fusion protein is fused to a peptide that inhibits the activity of a kinase. In some embodiments, the peptide inhibits the activity of protein Kinase A (PKA) or c-Jun N-terminal kinase (JNK). In some embodiments, the peptide comprises the sequence GRTGRRNAI (SEQ ID NO:36). In some embodiments, the peptide comprises the sequence RPKRPTTLNLF (SEQ ID NO:37).

### Nucleic Acids

Also provided herein are nucleic acids encoding the fusion proteins disclosed herein, as well as vectors, host cells, and expression systems.

The term “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

The nucleic acids encoding the fusion proteins disclosed may be, e.g., DNA, cDNA, RNA, synthetically produced DNA or RNA, or a recombinantly produced chimeric nucleic acid molecule comprising any of those polynucleotides either alone or in combination. For example, provided is an expression vector comprising a polynucleotide sequence encoding a fusion protein described herein operably linked to expression control sequences suitable for expression in a eukaryotic and/or prokaryotic host cell.

The term “vector” refers to a nucleic acid molecule capable of transporting another nucleic acid to which it has been linked. A “vector” includes, but is not limited to, a viral vector, a plasmid, an RNA vector or a linear or circular DNA or RNA molecule which may consists of a chromosomal, non-chromosomal, semi-synthetic or synthetic nucleic acids. In some embodiments, the employed vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g., adeno associated viruses, AAV), coronavirus, negative strand RNA viruses such as orthomyxovirus (e.g., influenza virus), rhabdovirus (e. g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus.

A variety of expression vectors have been developed for the efficient synthesis of fusion proteins in prokaryotic cells such as bacteria and in eukaryotic systems, including but not limited to yeast and mammalian cell culture systems have been developed. The vectors can comprise segments of chromosomal, non-chromosomal and synthetic DNA sequences. Also provided are cells comprising expression vectors for the expression of fusion proteins disclosed herein.

### Methods

Provided herein is a method of recoloring a cell expressing a first fluorescent protein, the method comprising:
(a) expressing an internal fusion protein in the cell, wherein the internal fusion protein comprises a nanobody into which a second fluorescent protein is inserted, wherein the nanobody binds to the first fluorescent protein;
(b) detecting the fluorescence of the second fluorescent protein.

Provided herein is a method of detecting the simultaneous presence of a first and a second antigen in a cell, the method comprising
(a) expressing in the cell a first internal fusion protein fused to a second internal fusion protein: wherein the first fusion protein comprises (i) a first nanobody that binds to the first antigen and (ii) a first fluorescent protein inserted into the first nanobody: and wherein the second fusion protein comprises (i) a second nanobody that binds to the second antigen and optionally (ii) a second fluorescent protein inserted into the second nanobody: and
(b) detecting the fluorescence of the first and optionally the fluorescence of the second fluorescent protein: wherein presence of fluorescence of the first and optionally of the second fluorescent protein indicates simultaneous presence of the first and the second antigen in the cell: and absence of fluorescence of the first and optionally of the second fluorescent protein indicates absence of the first and/or the second antigen in the cell.

Provided herein is a method of promoting degradation of a target antigen in a cell, the method comprising expressing in the cell an internal fusion protein comprising a first nanobody directed against a first antigen, wherein the fusion protein is fused to second nanobody directed against an antigen that is to be targeted for degradation (i.e., the second antigen): and wherein:
(a) in the presence of the first antigen, degradation of the second antigen is not promoted; and
(b) in the absence of the first antigen, degradation of the second antigen is promoted.

In one embodiment, a fluorescent protein is inserted into the first nanobody, and wherein:
(a) in the presence of the first antigen, the fluorescent protein emits fluorescence; and
(b) in the absence of the first antigen, the fluorescent protein does not emit fluorescence.

Provided herein is a method of regulating the expression of a protein of interest in a cell, the method comprising expressing in the cell an internal fusion protein comprising a nanobody directed against an antigen, wherein the fusion protein is fused to transcription factor that controls the expressing of the protein of interest, wherein:
(a) in the presence of the antigen, expression of the protein of interest is increased; and
(b) in the absence of the antigen, expression of the protein of interest is decreased.

In one embodiment, a fluorescent protein is inserted into the first nanobody, and wherein:
(a) in the presence of the first antigen, the fluorescent protein emits fluorescence; and
(b) in the absence of the first antigen, the fluorescent protein does not emit fluorescence.

Provided herein is a method of regulating kinase activity in a cell, the method comprising expressing an internal fusion protein comprising a nanobody directed against an antigen, wherein the internal fusion protein is fused to a peptide that inhibits the activity of a kinase and wherein:
(a) in the presence of the antigen, the activity of the kinase is inhibited; and
(b) in the absence of the antigen, the activity of the kinase is not inhibited.

In one embodiment, a fluorescent protein is inserted into the first nanobody, and wherein:
(a) in the presence of the first antigen, the fluorescent protein emits fluorescence; and
(b) in the absence of the first antigen, the fluorescent protein does not emit fluorescence.

Provided herein is a method of regulating kinase activity in a cell, the method comprising expressing an internal fusion protein comprising a nanobody directed against an antigen, wherein the internal fusion protein is fused to a peptide that reduces the activity of a kinase and wherein:
(a) in the presence of the antigen, the activity of the kinase is reduced; and
(b) in the absence of the antigen, the activity of the kinase is not reduced.

In one embodiment, a fluorescent protein is inserted into the first nanobody, and wherein:
(a) in the presence of the first antigen, the fluorescent protein emits fluorescence; and
(b) in the absence of the first antigen, the fluorescent protein does not emit fluorescence.

It is to be understood that this disclosure is not limited to the particular molecules, compositions, methodologies, or protocols described, as these may vary. Any methods and materials similar or equivalent to those described herein can be used in the practice or testing of embodiments of the disclosure. It is further to be understood that this disclosure includes all possible combinations of such particular features. For example, where a particular feature is disclosed in the context of a particular aspect or embodiment, or a particular claim, that feature can also be used, to the extent possible, in combination with and/or in the context of other particular aspects and embodiments.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the disclosure, the following examples of specific embodiments are given. The following examples should not be read to limit or define the entire scope of the disclosure.

### EXAMPLES

### Example 1: Materials and Methods for the Experiments Presented in Examples 2-21

### Mutagenesis and Directed Molecular Evolution

The genes encoding miRFPnano proteins and their mutants were amplified by polymerase chain reaction (PCR) and inserted into the pBAD/His-B vector (Invitrogen/Thermo-Fisher Scientific) at KpnI/EcoRI sites. All oligonucleotide PCR primers were purchased from Biomers.

For BV synthesis, E, coli host cells were cotransformed with a pWA23 h plasmid encoding heme oxygenase from Bradyrhizobium ORS278 under rhamnose promoter.

Random mutagenesis of genes encoding miRFPnanos was performed with a GeneMorph II random mutagenesis kit (Agilent Technologies). Site-specific mutagenesis and saturated mutagenesis were performed by overlap-extension PCR. For library construction, a mixture of mutated genes was cloned into pBAD/His-B vector and electroporated into LMG194 host cells (Invitrogen/Thermo-Fisher Scientific), containing a pWA23 h plasmid. Typical mutant libraries contained 10⁷-10⁸ clones.

Flow cytometry screening of mutant libraries was performed using an Influx cell sorter (BD Biosciences). 640 nm laser for excitation and 670/30 nm or 725/40 nm emission filters were used for the selection of positive clones. Before sorting, cells were grown in LB/ampicillin/kanamycin medium supplemented with 0.02% rhamnose and 0.005% arabinose for 5 h at 37° C., and then for 20 h at 22° C. The next day bacterial cells were pelleted, washed and diluted with phosphate-buffered saline (PBS) to an optical density of 0.03 at 600 nm. Cells collected after sorting were incubated in SOC medium for 1 h at 37° C., and then plated on LB/ampicillin/kanamycin Petri dishes supplemented with 0.005% arabinose and 0.02% rhamnose overnight at 37° C. Screening of brightest clones was performed with Leica M205 fluorescence stereomicroscope equipped with CCD camera

(Tucsen), using two filter sets: 650/45 nm excitation and 710/50 nm emission, and 700/20 nm excitation and 730 nm LP emission. About 30 selected clones were subcloned into a pcDNA3.1 plasmid (Invitrogen/Thermo-Fisher Scientific) and evaluated in transiently transfected HeLa cells.

### Protein Expression and Characterization

Proteins were expressed in LMG194 bacterial cells, cotransformed with pWA23 h plasmid, encoding heme oxygenase. Bacterial cells were grown to an optical density of 0.5-0.7 at 600 nm in LB/ampicillin/kanamycin medium supplemented with 0.02% rhamnose and, then, to induce miRFPnanos or NIR-Fb expression, 0.005% arabinose was added. Bacteria were cultured for 5 h at 37° C., and, then, at 22° C., for 20 h.

Protein purification was performed with Ni-NTA agarose (Qiagen). Proteins were eluted with PBS containing 100 mM EDTA.

Fluorescence spectra were recorded with a Cary Eclipse fluorimeter (Agilent Technologies). Absorbance measurements were performed with a Hitachi U-2000 spectrophotometer. The extinction coefficients of miRFPnanos were determined as a ratio between the absorbance value of the main peak at the Q-band and the value of the peak at the Soret band, assuming the latter to have the extinction coefficient of free BV of 39,900 M⁻¹ cm⁻¹. The fluorescence quantum yields of miRFPnanos were determined using a Nile blue dye and miRFP709 as standards.

The pH stability was studied using a series of buffers (100 mM sodium acetate, 300 mM NaCl for pH 2.5-5.0, and 100 mM NaH₂PO₄, 300 mM NaCl for pH 4.5-9.0).

Distance between the adjacent B-strands of an Nb after insertion of miRFP670nano3 was estimated with a Coot software (Crystallographic Object-Oriented Toolkit) using an Nb structure from the crystallized GFP:antiGFP-Nb complex (PDB ID: 3OGO).

### Protein Crystallization and Structure Solution

For crystallization, miRFP670nano3 and miRFP718nano were equilibrated in 20 mM Tris-HCl, 300 mM NaCl at pH 8.0 buffer and concentrated to 27.3 and 27.8 mg ml⁻¹, respectively. Initial crystallization conditions were found with the NT8 crystallization robot (Formulatrix) using Hampton Research, Jena Bioscience, and Molecular Dimensions screens. The conditions were further optimized with additive screens. The best crystals could be obtained from (8.4% PEG 4000, 3.6% MPD, 0.06 M sodium/potassium phosphate buffer pH 6.3) and (12.6% PEG 6000, 0.1 M lithium sulfate, 0.07 M citric acid buffer pH 3.5, 2.1% D-sorbitol) for miRFP670nano3 and miRFP718nano, respectively. The crystals suitable for X-ray data collection were grown by the hanging-drop vapor diffusion method. In the large-scale crystallization experiment, 2 μl of the protein solution was mixed with 2 μl of the reservoir solution and incubated against 500 ml of the same reservoir solution at 20° C. for a week.

X-ray data were acquired on SER-CAT 22-ID and 22-BM beamline stations (Advanced Photon Source, Argonne National Laboratory, Argonne, IL). Before data collection, the crystals were flash-frozen in a 100 K nitrogen gas stream. Diffraction images were processed with HKL200049. The structures were solved by the molecular replacement method with MOLREP50 using the structure of miRFP670nano (PDB ID: 6MGH) as a search model. To remove model bias, the structures were rebuilt with ARP/wARP model building and density improvement software. The structure refinement was carried out with REFMAC5 (CCP4 suite) and PHENIX.REFINE (PHENIX suite) programs. Realspace model correction and structure validation were performed with COOT.

### 703 Dot-Blot Assay

2 μl of EGFP or NIR-Fb_{GFP} and control antigen bovine serum albumin were spotted in PBS (proteins concentration 1 μ/ml) to the nitrocellulose membranes. The membranes were air-dried and incubated with 10% non-fat skimmed milk in PBS at 37° C., for 1 h. Then membranes were washed with PBS and incubated with EGFP or NIR-Fb_{GFP} (1:100 dilution, PBS, 0.05% Tween-20), respectively, at 37º C for 1 h. After incubation membraned were washed and imaged using the Leica M205 fluorescence stereomicroscope, using two filter sets: 650/45 nm excitation and 710/50 nm emission, for NIR-Fb_{GFP} and 480/40 nm excitation and 535/50 nm emission for EGFP.

### Construction of Mammalian Plasmids

To construct plasmids encoding miRFPnanos or their mutants, the respective genes were inserted into the pcDNA3.1 plasmid (Invitrogen/Thermo Fisher Scientific) at KpnI/EcoRI sites.

To engineer plasmids for protein tagging and labeling of intracellular structures, the miRFP670nano3 (GenBank miRFP670nano3), miRFP704nano (GenBank MW627295), or miRFP718nano (GenBank MW627296) genes were swapped with miRFP703 either as C- (for LAMP1 and H2B) or N-terminal fusions (for α-tubulin, β-actin, myosin and clathrin).

Nanobodies internally fused with miRFPnanos were generated by overlap PCR, and the resulted constructs were inserted into the pcDNA plasmid at the KpnI/EcoRI sites. Nb to GFP was amplified from a pcDNA3.1-NSImb-vhhGFP4 plasmid (Addgene #35579). Nb to actin was amplified from a plasmid commercially available from ChromoTek. Nb to mCherry was amplified from a pGEX6P1-mCherry-Nanobody plasmid (Addgene #70696). Nb to ALFA-tag was amplified from a pET51b(+)-EGFP-Nb_{ALFA} plasmid (Addgene #136626). Genes of Nb 2E7 to HIV gp41, Nb21 and Nbm6 to SARS-COV-2 to spike protein were synthesized by GenScript. A NIR-Fb_{actin}/Y58C-EGFP fusion was generated by overlap PCR and then a Tyr58Cys mutation was introduced to miRFP670nano3 by site-directed mutagenesis.

The gene encoding RBD (333-529) of SARS-COV-2 spike protein was amplified from a pDONR223-SARS-COV-2 S plasmid (Addgene #149329). The gene encoding human β-catenin was amplified from a pCI-neo-β-catenin plasmid (Addgene #16518). The gene encoding HIV p24 was amplified from a pET51b(+)-SNAP-p24 plasmid (Addgene #130718). The gene encoding DHFR was amplified from a pET22b-ecDHFR plasmid (Addgene #109055). The gene encoding HIV gp41 was amplified from a pLAI-Env plasmid (Addgene #133996). The PCR-amplified genes were then fused with msfGFP by SOE-PCR and inserted into a pcDNA3.1 plasmid. ALFA-fused α-tubulin, β-actin, myosin, and clathrin were generated by PCR with primer encoding ALFA-tag.

To generate NIR-Fb fusions, a 20 amino acid linker (Gly₄Ser)₄ (SEQ ID NO:41) was inserted into the pcDNA plasmid by the BamHI/NotI sites. For the construction of bispecific NIR-Fbs, NIR-Fb_{GFP} was inserted into the pcDNA3.1 plasmid, containing the (Gly₄Ser)₄ (SEQ ID NO:41) linker at the KpnI/BamHI sites, and NIR-Fb_{mCherry} was inserted at the NotI/XbaI sites. To generate a NIR-Fb_{mCherry}-EGFP fusion, EGFP was inserted into the pcDNA3.1 plasmid, containing a linker (Gly₄Ser)₄ and NIR-Fb_{mCherry} at NotI/XbaI site.

To engineer a NIR-Fb_{GFP}-GAL4 fusion, the GAL4-VP16 sequence was PCR-amplified from a pGV-ER plasmid (Systasy) and inserted at the NotI/XbaI sites into the pcDNA3.1 plasmid containing NIR-Fb_{GFP} and linker (Gly₄Ser)₄. To generate a NIR-Fb_{GFP}-Nb_{ALFA}, the NIR-Fb_{GFP} was inserted into the pcDNA-3.1 plasmid, containing the (Gly₄Ser)₄ linker at the KpnI/BamHI sites, and Nb_{ALFA} was inserted at the NotI/XbaI sites. To generate biNIR-Fb_{GFP}-GAL4 and biNIR-Fb_{GFP}-Nb_{ALFA} fusions, two NIR-Fb_{GFP} genes were joined by SOE-PCR and swapped with NIR-Fb_{GFP}. The ALFA-tag was added to the N-terminus of GAL4-VP16 with oligonucleotide primer and inserted into the pcDNA3.1 plasmid, at the KpnI/XbaI site.

To generate fusions with inhibitory peptides, the peptide sequences were introduced to the NIR-Fb_{GFP} with the (Gly₄Ser)₄ linker by PCR, and a Y58C mutation was introduced to miRFP670nano3 by overlap PCR.

To construct Gluc-biNIR-Fb_{GFP} fusion Gluc gene was amplified from pUAS-Gluc plasmid and inserted to plasmid encoding biNIR-Fb_{GFP} at the HindIII/KpnI sites. To generate a biNIR-Fb_{GFP}-caspase/V266E and biNIR-Fb_{GFP}-DTA plasmids the biNIR-Fb_{GFP} was inserted into the pcDNA-3.1 plasmid, containing the (Gly₄Ser)₄ linker at the KpnI/BamHI sites, and caspase/V266E or DTA, respectively, were inserted at the NotI/XbaI sites. The gene encoding caspase/V266E was amplified from a pET21b-Caspase-3(V266E) plasmid (Addgene #90089). The gene encoding DTA was amplified from a pAAV-mCherry-flex-dtA plasmid (Addgene #58536).

### Mammalian Cells and Transfection

HeLa (CCL-2), N2A (CCL-131), U-2 OS (HTB-96), HEK293T (CRL-3216), and NIH3T3 (CRL-1658) cells were obtained from the ATCC.

Cells were cultured in a DMEM medium supplemented with 10% FBS, 0.5% penicillin-streptomycin and 2 mM glutamine (Invitrogen/Thermo-Fisher Scientific) at 37° C.

For live-cell fluorescence microscopy, cells were plated in 35 mm glass-bottom Petri dishes (Greiner Bio-One International). Transient transfections were performed using polyethyleneimine or Effectene Transfection Reagent (Qiagen).

### Neuronal Culture and Transfection

Primary rat cortical neurons were prepared in the Neuronal Cell Culture Unit, University of Helsinki. All animal work was performed under the ethical guidelines of the European convention and regulations of the Ethics Committee for Animal Research of the University of Helsinki. Cells were plated at a density of 500,000-700,000 per 35 mm glass-bottom dish, coated with Poly-L-Lysine (0.01 mg/ml) (Merck). Neurons were grown at 37° C. and 5% CO2 in neurobasal medium (Gibco) supplemented with B27 (Invitrogen/Thermo-Fisher Scientific), L-glutamine (Invitrogen/Thermo-Fisher Scientific), and penicillin-streptomycin (Lonza). Cultured neurons were transfected with pcDNA plasmids encoding respective miRFPnano at 4-5 days in vitro (DIV) using Effectene Transfection Reagent (Qiagen) and imaged 48-72 h after transfection.

### Wide-Field Fluorescence Microscopy

Live cells were imaged with an Olympus IX81 inverted epifluorescence microscope, equipped with a Xenon lamp (Lambda LS, Sutter). An ORCA-Flash4.0 V3 camera (Hamamatsu) was used for image acquisition. Cells were imaged using either a 20×0.75 NA air or a 60×1.35 NA oil objective lens (UPlanSApo, Olympus). During imaging, HeLa cells were incubated in a cell imaging solution (Life Technologies-Invitrogen) and kept at 37° C. The microscope was operated with a SlideBook v.6.0.8 software (Intelligent Imaging Innovations). To separately image miRFP670nano3 and miRFP18nano, two filter sets (605/30 nm exciter with 667/30 nm emitter, and 685/20 nm exciter with 725/40 nm emitter) (Chroma) were used. The data were analyzed using a SlideBook v. 6.0.8 (Intelligent Imaging Innovations) and a Fiji v. 1.50b software.

Photobleaching measurements were performed in live HeLa cells 48 h after the transfection using 60×1.35NA oil objective lens (UPlanSApo, Olympus). Obtained raw data were normalized to corresponding absorbance spectra and extinction coefficients of the miRFPnanos, the spectrum of Xenon lamp and the transmission of the 665/45 nm photobleaching filter.

### Flow Cytometry

Flow cytometry analysis was performed using an Accuri C6 flow cytometer (BD Biosciences). Before analysis, live cells were washed and diluted in cold PBS to a density of 500,000 cells per ml. At least 50,000 cells per sample were recorded. The fluorescence intensity of miRFP670nano, miRFP670nano3, miRFP704nano and miRFP718nano expressing cells was analyzed using the 640 nm excitation laser and 675/25 nm or 670 nm LP emission filters. The fluorescence intensity of EGFP was analyzed using 488 nm laser for excitation, and its fluorescence was detected with a 510/15 nm emission filter. The data were analyzed using FlowJo v.7.6.2 software.

### Gaussia Luciferase Assay

For an antigen-dependent GAL4 stabilization, Hela cells were co-transfected with plasmids encoding NIR-Fb_{GFP}-GAL4 or biNIR-Fb_{GFP}-GAL4, reporter plasmid pUAS-Gluc (5×UAS), and with either EGFP or mTagBFP in a 1:10:89 ratio. HeLa cells were co-transfected with plasmids encoding ALFA-tagged GAL4-VP16, reporter plasmid pUAS-Gluc (5×UAS), NIR-Fb_{GFP}-Nb_{ALFA} or biNIR-Fb_{GFP}-Nb_{ALFA}, and with either EGFP or mTagBFP2 in the 1:10:44.5:44.5 ratio. To measure Gluc activity, the co-transfected Hela cells were grown in 24-well plates. 10 h after the transfection mixture was removed, a fresh growth medium was added, and cells were incubated for 48 h. Then, 5 μl of culture media was mixed with 100 μl of 2 μM coelenterazine (Invitrogen/Thermo Fisher Scientific) in PBS in wells of a 96-well half-area white plate (Costar). Bioluminescence was measured using a Victor X3 multilabel plate reader (PerkinElmer).

### Cell Viability Assays

The viability of cells transfected with caspase/V266E and biNIR-Fb_{GFP}-caspase/V266E fusion was monitored by Annexin V staining (Annexin V-AlexaFluor555 conjugate: Invitrogen/Thermo Fisher Scientific). The viability of cells transfected with DTA and biNIR-Fb_{GFP}-DTA fusion was quantified by MTT test. Transfection efficiency was measured by flow cytometry and signal of non-transfected cells was subtracted.

### Viral Vector Production and Stereotactic Injection.

The miRFP670nano3 gene was PCR amplified and subcloned into an AAV transfer vector downstream of the human synapsin promoter and upstream of WPRE and hGHpA sequences. This vector was co-transfected into HEK293-AAV cells (Vector Biolabs) along with a pAdeno-helper vector and a pRC-AAV9 rep-cap plasmid. Recombinant AAV9 production was then carried out using a protocol developed by the Byungkook Lim laboratory at the University of California at San-Diego. The recombinant AAV9-hSYN-miRFP670nano3-WPRE-hGHpA was titered by qPCR using primers designed to the hGHpA sequence. The titer of the virus was 1.4E+12 GC ml⁻¹. The vector (volume: 0.4 l:μ undiluted) was injected into the cortex (coordinates: AP −0.8-(−1.75) mm, ML 1.45-1.65 mm, DV 0.25-1.1 mm) or dorsal horn of the L3-L4 spinal cord (ML 0.3, DV 0.3-0.5 mm). For surgical procedures, thin-wall glass pipettes were pulled on a Sutter Flaming/Brown micropipette puller (model P-97). Pipette tips were cut at an acute angle under 10× magnification using sterile techniques. Tip diameters were typically 15-20 μm. Pipettes that did not result in sharp bevels nor had larger tip diameters were discarded. Millimeter tick marks were made on each pulled needle to measure the virus volume injected into the brain or spinal cord.

Mice were anesthetized with isoflurane (4% for induction: 1%-1.5% for maintenance) and positioned in a computer-assisted stereotactic system with digital coordinate readout and atlas targeting (Leica Angle Two). Body temperature was maintained at 36-37° C., with a direct current (DC) temperature controller and ophthalmic ointment was used to prevent eyes from drying. A small amount of depilator cream (Nair) was used to remove hair over the dorsal areas of the injection site thoroughly. The skin was cleaned and sterilized with a two-stage scrub of betadine and 70% ethanol.

For brain injections, a midline incision was made beginning just posterior to the eyes and ending just passed the lambda suture. The scalp was pulled open and periosteum cleaned using scalpel and forceps to expose the desired hemisphere for calibrating the digital atlas and coordinate marking. Once reference points (bregma and lambda) were positioned using the pipette needle and entered into the program, the desired target was set on the digital atlas. The injection pipette was carefully moved to the target site (using AP and ML coordinates). Next, the craniotomy site was marked and an electrical micro-drill with a fluted bit (0.5 mm tip diameter) was used to thin a 0.5-1 mm diameter part of the bone over the target injection site. Once the bone was thin enough to flex gently, a 30G needle with an attached syringe was used to carefully cut and lift a small (0.3-0.4 mm) segment of bone.

For spinal cord injections, surgical scissors were used to make a small (around 10 mm) incision along the midline. Fascia connecting the skin to the underlying muscle was removed with forceps. The skin was held back by retractors. Using blunt dissection, lateral edges of the spinal column were isolated from connective tissue and muscle. Tissue from the vertebra of interest and one vertebra rostral and caudal to the site of spinal cord exposure was removed with forceps. The spine was then stabilized using Cunningham vertebral clamps and any remaining connective tissue on top of the exposed vertebrae removed with a spatula. Using a small sterile needle, an approximately 0).3 mm opening was made in the tissue overlying the designated injection site.

For injection, a drop of the virus was carefully pipetted onto parafilm (1-2 □l) for filling the pulled injection needle with the desired volume. Once loaded with sufficient volume, the injection needle was slowly lowered into the brain or spinal cord until the target depth was reached. Manual pressure was applied using a 30-ml syringe connected by shrink tubing and 0.4 μl of the virus was slowly injected over 5-10 min. Once the virus was injected, the syringe's pressure valve was locked. The position was maintained for approximately 10 min to allow the virus to spread and to avoid backflow upon needle retraction. Following the injection, head or spinal cord clamps were removed, muscle approximated, and the skin sutured along the incision. Mice were given subcutaneous Buprenex SR (0).5 mg per kg) and allowed to recover before placement in their cage.

### Animal Preparation for In Vivo Two-Photon Imaging

All live animal procedures were performed following the guidelines of the National Institutes of Health and were approved by the Institutional Animal Care and Use Committee at the Salk Institute. Male Cx3cr1^{GFP/+} mice (stock #005582, Jackson Laboratories) with an age of 8-14 weeks at the time of imaging (6-9 weeks at the time of stereotactic injection) were used.

For surgical procedures, mice were anesthetized with isoflurane (4-5% for induction: 1%-1.5% for maintenance) and implanted with a head or spinal plate on a custom surgical bed (Thorlabs). Body temperature was maintained at 36-37° C. with a DC temperature control system and ophthalmic ointment was used to prevent eyes from drying. Depilator cream (Nair) was used to remove hair above the imaging site. The skin was thoroughly cleansed and disinfected with a two-stage scrub of betadine and 70% ethanol.

For brain imaging, a scalp portion was surgically removed to expose frontal, parietal, and interparietal skull segments. Scalp edges were attached to the lateral sides of the skull using a tissue-compatible adhesive (3M Vetbond). A custom-machined metal plate was affixed to the skull with dental cement (Coltene Whaledent, cat, no. H00335), allowing the head to be stabilized with a custom holder. An approximately 3 mm diameter craniotomy was made over the AAV injection site. A 1.5% agarose solution and coverslip were applied to the exposed tissue, and a coverslip affixed to the skull with dental cement to control tissue motion. Imaging commenced immediately after optical window preparation.

For spinal cord imaging, a laminectomy was performed over the AAV injection site. The dura mater overlying the spinal cord was kept intact. A custom-cut #0 coverslip was placed over the exposed tissue and fixed to the spine with dental cement to control tissue motion. The depth of anesthesia was monitored throughout the experiment and adjusted as needed to maintain a breath rate of approximately 55-65 breaths per minute. Saline was supplemented as needed to compensate for fluid loss.

### Two-Photon Microscopy

Live animal imaging was performed 2-5 weeks after AAV injection. Briefly, a Sutter Movable Objective Microscope equipped with a pulsed femtosecond Ti:Sapphire laser (Chameleon Ultra II, Coherent) with two fluorescence detection channels was used for imaging (green emission filter: ET-525/70M (Chroma); near-infrared emission filters: ET645/75M (Chroma) in conjunction with FF01-720/SP (Semrock); dichroic beamsplitter: 565DCXR (Chroma); photomultiplier tubes: H7422-40 GaAsP (Hamamatsu)). The laser excitation wavelength was set between 880-930 nm. The average laser power was <10-15 mW at the tissue surface and adjusted with depth as needed to compensate for signal loss due to scattering and absorption. An Olympus 20×1.0 NA water immersion objective was used for light delivery and collection. Z-stacks included up to 900 images, acquired at 1-2 μm axial step size, used a two- to four-frame average, 256 or 512×512-pixel resolution, and 1.0×-2.5× zoom (corresponding to 701-282 μm fields of view).

### In Vivo Image Data Processing

Data were processed in ImageJ or Fiji software (SciJava, https://fiji.sc) using the ‘Remove Outliers’ (radius: 1 pixel: threshold: 75) and ‘Subtract Background’ (rolling ball radius: 256 pixels) functions, xz projections were created from xy fluorescence image stacks using the ‘Reslice’ and ‘3D Project’ functions on 15 μm-sized sub-volumes. Motion artifacts in spinal cord images were reduced using the ‘Image Stabilizer’ plugin (transformation: affine).

### Example 2: Identification of Engineered Fluorescent Proteins with Enhanced Fluorescent Properties

To generate fluorescent protein (FP) variants with improved properties, miRFP670nano (see Table 1) was used as a starting point. miRFP670nano is a single-domain fluorescent protein that uses biliverdin IVa as a chromophore. In miRFP670nano, the chromophore forms a covalent thioether bond between the C3¹ atom of ring A of BV and Cys86 residue. The formation of the covalent bond is accompanied by a reduction of ring A, shortening of the conjugated system of the chromophore and, hence, resulting in a hypsochromic shift of its fluorescence. In the cystral structure of miRFP670nano, the C^{β} and C^{γ} atoms of Arg57 are positioned close to the C32 atom of ring A of BV.

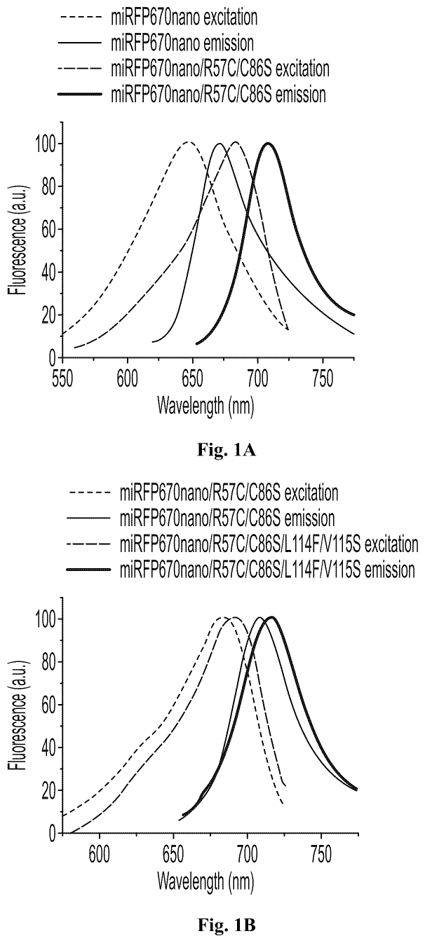

To enable a covalent binding of BV via the C32 atom, resulting in spectral red-shift, a R57C C86S double mutant of miRFP670nano was generated (see Table 1). Indeed, the fluorescence spectra for miRFP670nano R57C C86S double mutant were red-shifted by 35 nm as compared to parental miRFP670nano (FIG. 1A).

To red-shift the spectra further, saturated mutagenesis of the miRFP670nano R57C C86S double mutant was performed at residues 113-117, which surround chromophore ring D. An even more red-shifted variant with two extra mutations L114F and V115S was identified (miRFP670nano R57C C86S L114F V115S). (FIG. 1B). Parental miRFP670nano as well as miRFP670nano R57C C86S and miRFP670nano R57C C86S L114F V115S were subjected to several rounds of random mutagenesis. Positions identified by random mutagenesis were then targeted in each protein separately using saturated mutagenesis. To identify fluorescent proteins that would provide bright fluorescence in mammalian cells FPS, clones were screened in each round not only in E, coli, but also in transiently transfected HeLa cells. After 15 rounds of molecular evolution resulted three mutant FPs, were isolated: miRFP670nano3 (14 mutations as compared to parental FP miRFP670nano), miRFP704nano (14 mutations as compared to parental FP miRFP670nano), and miRFP718nano (26 amino acid mutations as compared to parental FP miRFP670nano). See Table 1.

TABLE 1
FP variants

| SEQ | | |
|---|---|---|
| ID | Amino acid sequence | Fluorescent |
| NO | SEQ ID NO | protein |
| 1 | MANLDKMLNTTVTEVRQFLQVDRVCVFQFEE | miRFP670nano |
| | DYSGVVVVEAVDDRWISILKTQVRDRYFMET | |
| | RGEEYSHGRYQAIADIYTANLTECYRDLLTQ | |
| | FQVRAILAVPILQGKKLWGLLVAHQLAAPRQ | |
| | WQTWEIDFLKQQAVVVGIAIQQS | |
| 2 | MANLDKMLNTTVTEVRQFLQVDRVCVFQFEE | miRFP670nano |
| | DYSGVVVVEAVDDRWISILKTQVRDCYFMET | R57C C86S |
| | RGEEYSHGRYQAIADIYTANLTESYRDLLTQ | |
| | FQVRAILAVPILQGKKLWGLLVAHQLAAPRQ | |
| | WQTWEIDFLKQQAVVVGIAIQQS | |
| 3 | MANLDKMLNTTVTEVRQFLQVDRVCVFQFEE | miRFP670nano |
| | DYSGVVVVEAVDDRWISILKTQVRDCYFMET | R57C C86S |
| | RGEEYSHGRYQAIADIYTANLTESYRDLLTQ | |
| | FQVRAILAVPILQGKKLWGLFSAHQLAAPRQ | L114F V115S |
| | WQTWEIDFLKQQAVVVGIAIQQS | |
| 4 | MANLDKMLNTTVTEVRKFLQADRVCVFKFEE | miRFP670nano |
| | DYSGTVSHEAVDDRWISILKTQVQDRYFMET | 3 |
| | RGEEYVHGRYQAIADIYTANLVECYRDLLIE | |
| | FQVRAILAVPILQGKKLWGLLVAHQLAGPRE | |
| | WQTWEIDFLKQQAVVMGIAIQQS | |
| 5 | MANLDKMLNTIVTEVRQFLQVDRVCVFQFEE | miRFP704nano |
| | DYSGSVVVEAVDDRWNSILKTQVRDCYFMET | |
| | RGEEYLHGRYQAIADIYQANLLESYRDLLGQ | |
| | FQVRAILAVPIIKGKKLWGLLVAHQLAAPRS | |
| | WQTWEIEFLKQQAVVMGIAIQQS | |
| 6 | MANLDKMLNTIVTEVRQFLQVDRLCVFKFEE | miRFP718nano |
| | DYSGNIIYEAVDDGWLSILKTHVRDCYFMET | |
| | RGEEYLHGRYQAIADIHQANLAESYRDFLTQ | |
| | YQVRAIVAVPILKGKKLWGLFSAHQLAAPRS | |
| | WQAWEIEFLKQQAVVMGIAIQQS | |
| 7 | MANLDKMLNTTVTEVRKFLQADRVCVFKFEE | Non- |
| | DYSGTVSHEAVDDRWISILKTQVQDRCFMET | fluorescent |
| | RGEEYVHGRYQAIADIYTANLVECYRDLLIE | miRFP670nano |
| | FQVRAILAVPILQGKKLWGLLVAHQLAGPRE | 3 variant |
| | WQTWEIDFLKQQAVVMGIAIQQS | Tyr58Cys |

### Example 3: Characterization of Engineered miRFPnano Variants In Vitro

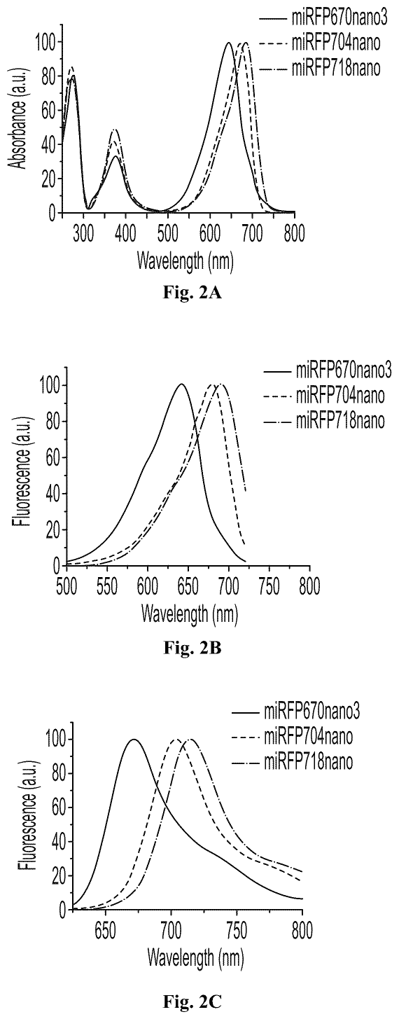

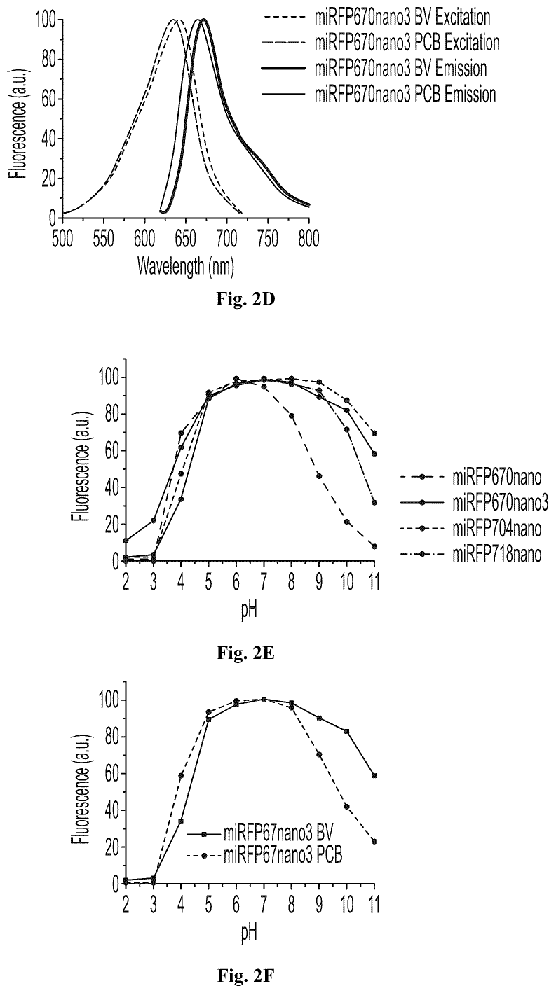

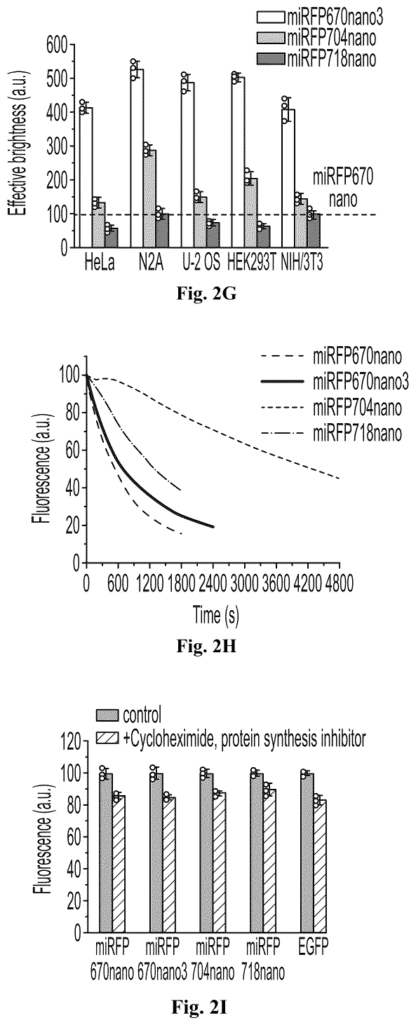

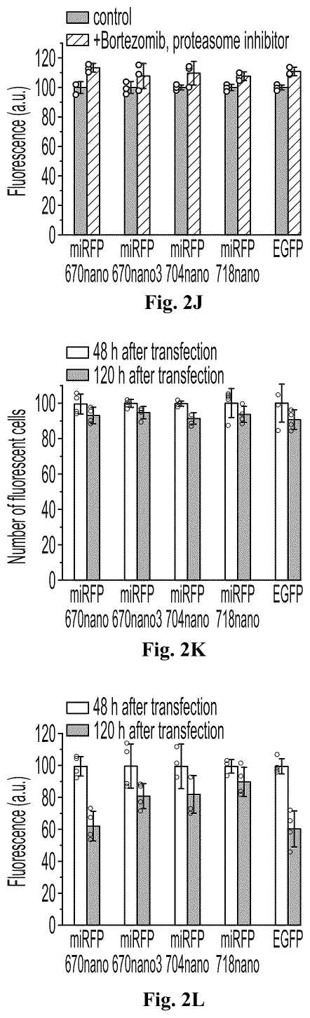

Single-domain 17 kDa miRFP704nano and miRFP718nano red-shifted NIR FPs exhibited excitation/emission maxima at 680/704 nm and 690/718 nm, respectively. Enhanced miRFP670nano3 protein with excitation/emission at 645/670 nm exhibited spectral properties similar to those of parental miRFP670nano (FIGS. 2A-C and Table 2). Molecular brightness (defined as the product of a molar extinction coefficient and a quantum yield) of miRFP704nano and miRFP718nano was similar or higher than that of spectrally comparable two-domain NIR FPs derived from bacterial phytochrome photoreceptors (BphPs). miRFP670nano3 exhibited 2-fold higher molecular brightness than parental miRFP670nano, and 1.5-fold higher than widely used mCherry or mCardinal. Notably, the extinction coefficient (129,000 M⁻¹ cm⁻¹) and fluorescence quantum yield (18.5%) of miRFP670nano3 are the highest among all NIR FPs with BV chromophore (see Table 2).

In addition to BV, miRFP670nano3 is also capable of binding a phycocyanobilin (PCB) tetrapyrrole, which is a natural chromophore for wild-type CBCRs27. The PCB chromophore increased miRFP670nano3 quantum yield to 22.4% (FIG. 2D and Table 2). The fluorescence of miRFPnanos was stable over a wide pH range of 4.0-11.0. (FIGS. 2E and 2F). In contrast to parental miRFP670nano, which is alkali-intolerant, miRFP670nano3, miRFP704nano, and miRFP718nano demonstrated high tolerance to both acidic and alkaline environments.

References listed in Table 2: (1) Shcherbakova, D. M., Stepanenko, O. V., Turoverov, K. K. & Verkhusha, V. V. Near-Infrared Fluorescent Proteins: Multiplexing and Optogenetics across Scales. Trends Biotechnol 36, 1230-1243 (2018); (2) Chernov, K. G., Redchuk, T. A., Omelina, E. S. & Verkhusha, V. V. Near-Infrared Fluorescent Proteins, Biosensors, and Optogenetic Tools Engineered from Phytochromes. Chem Rev 117, 6423-6446 (2017); (3) Snapp, E. Design and use of fluorescent fusion proteins in cell biology. Curr Protoc Cell Biol Chapter 21, 21 24 21-21 24 13 (2005); (4) Bathula, N. V., Bommadevara, H. & Hayes, J. M. Nanobodies: The Future of Antibody-Based Immune Therapeutics. Cancer Biother Radiopharm (2020); (5) Akiba, H, et al. Structural and thermodynamic basis for the recognition of the substrate-binding cleft on hen egg lysozyme by a single-domain antibody. Sci Rep 9, 15481 (2019); (6) Deschaght, P, et al. Large Diversity of Functional Nanobodies from a Camelid Immune Library Revealed by an Alternative Analysis of Next-Generation Sequencing Data. Front Immunol 8, 420 (2017); (7) Gonzalez-Sapienza, G., Sofia, M. A. R. & Rosa, T.-d. Single-Domain Antibodies As versatile Affinity Reagents for Analytical and Diagnostic Applications. Frontiers in Immunology 8 (2017); (8) Cheloha, R. W., Harmand, T. J., Wijne, C., Schwartz, T. U. & Ploegh, H. L. Exploring cellular biochemistry with nanobodies. J Biol Chem 295, 15307-15327 (2020).

TABLE 2
Properties of monomeric NIR FPs designed from various bacterial photoreceptors
and commonly used mCherry and mCardinal red-shifted GFP-like FPs.

| | | | | | Molecular | | | | Brightness in | |
|---|---|---|---|---|---|---|---|---|---|---|
| | | | | | brightness | | | | mammalian | |
| | | | Extinction | Quantum | vs. | | | Photostability | cells vs. | |
| | Ex | Em | coefficient, | yield | miRFP670 | pKa₁ | pKa₂ | in HeLa | miRFP670 | Ref |
| NIR FP | [nm] | [nm] | [M⁻¹ cm⁻¹] | [%] | nano [%] | (acid) | (alkali) | cells, t_{1/2}, s | nano, % a | (see text) |
| miRFP670 | 645 | 670 | 95,000 | 10.8 | 100 | 3.7 | 8.0 | 545 | 100 | 1 |
| nano | | | | | | | | | | |
| miRFP670 | 645 | 670 | 129,000 | 18.5 | 233 | 4.2 | 11 | 675 | 412 | U.S. |
| nano3 (BV) | | | | | | | | | | provisional |
| miRFP670 | 630 | 660 | 115,600 | 22.4 | 252 | 4.0 | 9.8 | n.a. | n.a. | application |
| nano3 (PCB) | | | | | | | | | | no. 17,115/223 |
| miRFP704 | 680 | 704 | 93,000 | 9.9 | 90 | 4.1 | >11 | 4265 | 134 | |
| nano | | | | | | | | | | |
| miRFP718 | 690 | 718 | 79,000 | 5.6 | 43 | 3.8 | 10.5 | 885 | 55 | |
| mano | | | | | | | | | | |
| miRFP670 | 642 | 670 | 87,400 | 14.0 | 119 | 4.5 | 9.5 | 183 | 117 | 2 |
| miRFP703 | 674 | 703 | 90,900 | 8.6 | 76 | 4.5 | >9.5 | 394 | 61 | |
| miRFP709 | 683 | 709 | 78,400 | 5.4 | 41 | 4.5 | 9.2 | 192 | 42 | |
| SNIFP | 697 | 720 | 75,000 b | 2.2 | 16 | 4.5 | >9.0 | n.a. | n.a. | 3 |
| mIFP | 683 | 704 | 82,000 | 8.4 | 67 | 4.5 | 9.2 | 54 | 26 | 4, 5 |
| BDFP1.5 | 688 | 711 | 74,000 | 5.0 | 36 | 2.0 | >10 | 1310 | 0.5 | 6 |
| mCherry | 587 | 610 | 72,000 | 122.0 | 154 | 4.5 | n.d. | 68 | n.d. | 7 |
| mCardinal | 604 | 659 | 87,000 | 19.0 | 160 | 5.3 | n.d. | 730 | n.d. | 8 |

### Example 4: Performance of Engineered miRFPnano Variants in Live Mammalian Cells

Engineered miRFPnanos variants were brightly fluorescent in mammalian cells without adding exogenous BV. The cellular (a.k.a, effective) brightness of miRFP670nano3 was >4-fold higher than that of miRFP670nano in all tested mammalian cell lines (FIG. 2G and Table 2). miRFP704nano was >2-fold brighter than spectrally close BphP-derived miRFP703, while miRFP718nano was 1.3-fold brighter than BphP-derived miRFP709 (Table 2).

miRFP670nano3 exhibited photostability close to that of miRFP670nano (which already has increased photostability as compared to BphP-derived NIR FPs) while the photostability of miRFP704nano and miRFP718nano were even 8.8- and 1.8-fold higher than that for parental miRFP670nano, respectively (FIG. 2H and Table 2).

Further, the engineered miRFPnanos exhibited high protein stability in mammalian cells. After a 4 h incubation with cycloheximide, a protein synthesis inhibitor, cells expressing engineered miRFPnanos retained 85-90% of their fluorescence (FIG. 2I). Conversely, the treatment with bortezomib, an inhibitor of proteasome-dependent protein degradation, increased the brightness of the miRFPnanos expressing cells only by 7-10% (FIG. 2H). Similar cellular stability was observed for miRFP670nano and EGFP (FIGS. 2I and 2J). High cellular stability was also confirmed by comparing cells transiently expressing engineered miRFPnanos 48 h and 120 h after transfection (FIG. 2K). Notably, 120 h after transfection, cells expressing the engineered miRFPnanos retained ˜80% of their fluorescence, while cells expressing miRFP670nano or EGFP retained only ˜60% of their fluorescence (FIG. 2L).

### Example 5: Engineered miRFPnano Variants Perform Well in Fusion Constructs and in Labeling Intracellular Structures

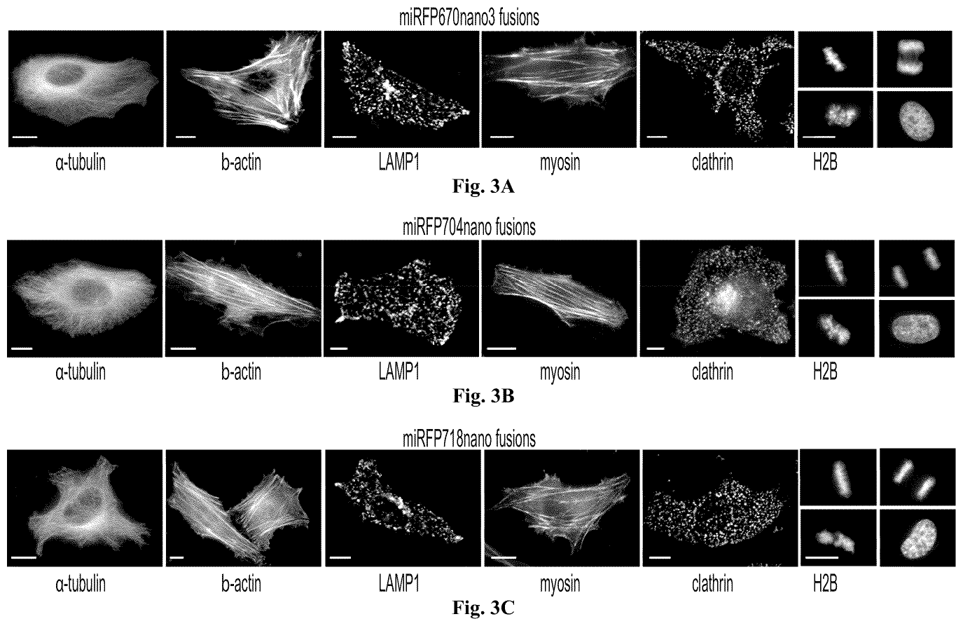

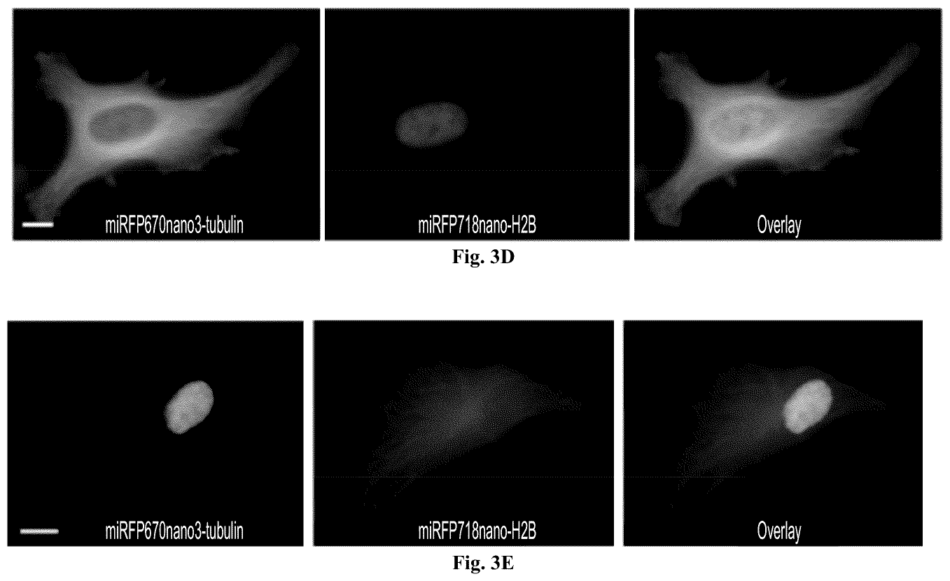

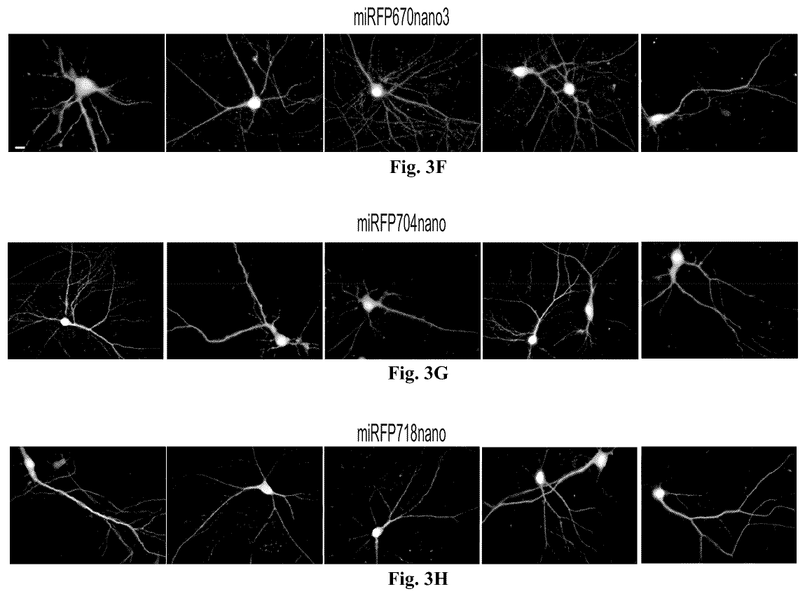

Due to their high brightness, pH-stability, and compact single-domain fold, the engineered miRFPnanos are well suited for their use in N- or C-terminal fusion constructs and for labeling intracellular structures. FIGS. 3A-C show results for miRFP670nano3, miRFP704nano, or miRFP718nano with α-tubulin, β-actin, myosin, or vesicular protein clathrin (-terminal fusions) or lysosomal membrane glycoprotein LAMP1 and histone H2B (C-terminal fusions).

In two-color NIR imaging, different co-expressed fusions of miRFP670nano3 or miRFP718nano had proper localization and clear separation of miRFP670nano3 and miRFP718 fluorescence signals (FIGS. 3D and 3E).

### Example 5: Engineered miRFPnano Variants Show Bright Fluorescence in Neurons

Primary rat cortical neurons expressing engineered miRFPnanos miRFP670nano3, miRFP704nano, or miRFP718nano exhibited bright homogenous fluorescence without the supply of exogenous BV (FIGS. 3F-H).

### Example 6: Structural Basis for the Properties of Engineered miRFPnanos

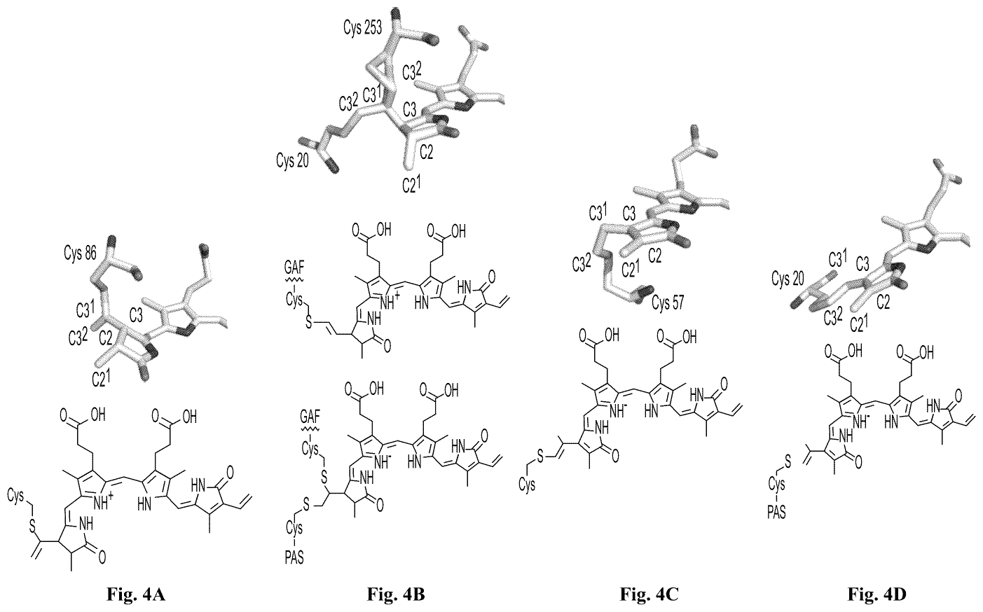

To investigate the structural basis for the changes in the biophysical and biochemical properties of the new miRFPnanos, the crystal structures of miRFP670nano3 and miRFP718nano were determined at 1.8 Å and 1.7 Å resolution, respectively. The crystal structures were compared with the structures of parental miRFP670nano, with which miRFP670nano3 and miRFP718nano share ˜ 80% of the sequence identity (FIG. 4). All three proteins have a similar GAF fold with the rigid N- and C-terminal helices positioned close to each other. This fold is similar to that of the GAF domains of miRFP670 and miRFP709 linked to the adjacent PAS domain (FIGS. 4B vs. 4D) that is absent in miRFPnanos derived from a single GAF-domain CBCR6.

miRIP670nano3

In miRFP670nano3, the chromophore BV is covalently attached by a thioether bond between the Cys86 residue and the C31 atom of the ring A, similar to the orientation in the parent miRFP670nano. The chromophore of miRFP670nano3 is stabilized by eight hydrogen bonds with the nearby residues Asp56, Tyr67, Arg71, Val84, and His117, a face-to-face stacking with Tyr87, and an edge-to-face stacking with Phe59.

The substantially increased quantum yield and brightness of miRFP670nano3 are likely provided by three mutations Val38Ser, Val39His, and Val140Met. The side chain of Ser38 forms a strong H-bond with the side chain of His39 locking it in the conformation favoring H-bonding with the side chain of Asp5. This H-bond stabilizes the spatial position of the N-terminal a-helix, tethering it to the second β-strand of the protein. The long side-chain of Met140 is the central element of the dense hydrophobic cluster formed by residues Leu8, Thr11, Val26, Ile104, Leu114, and Met140. This cluster effectively interconnects N- and C-terminal α-helices to the central β-sheet of the protein, making the fold of miRFP670nano3 more rigid than in parental miRFP670nano. The enhanced rigidity of the protein restricts BV mobility, makes the non-radiative transition of the chromophore less favorable, and hence provides for increased quantum yield and brightness of miRFP670nano3.

miRFP718nano

In miRFP718nano, the chromophore that forms a covalent thioether bond between the C32 atom of BV ring A and the rationally introduced Cys57 (FIG. 4C). The chromophore environment of miRFP718nano and miRFP670nano3 is similar but not the same. In addition to eight hydrogen bonds and two stacking interactions common for both proteins, miRFP718nano has an extra H-bond between ring D of BV and Ser115 (Val115 in miRFP670nano3) and the third tilted face-to-edge stacking between ring D and Phe90 (Leu90 in miRFP670nano3). It is likely H-bond with ring D and additional stacking interactions is mainly responsible for the bathochromic shift of miRFP718nano emission relative to that of miRFP704nano. Similarly, to His39 in miRFP670nano3, Tyr39 in miRFP718nano restrains the movement of N-terminal a-helix by forming an H-bond with Asp5. Stabilizing hydrophobic cluster interconnecting N- and C-terminal α-helixes with the central β-sheet of the protein is reinforced by substitutions Thr11Ile, Val24Leu, and Leu114Phe. The interactions between β-strands two and three are further strengthened by hydrogen bonds between residues Glu30, Asn36, and His53 that contribute to the enhanced rigidity of a single GAF domain protein scaffold.

### Example 7: Generation of Near-Infrared Fluorescent Nanobodies (NIR-Fbs)

An internal fusion of an engineered miRFPnano variant, here miRFP670nano3, with a well-characterized nanobody (Nb) directed against green fluorescent protein (Nb_{GFP}) was generated. Three insertion sites for miRFP670nano3 in Nb_{GFP} were tested (FIGS. 5A and 5B).

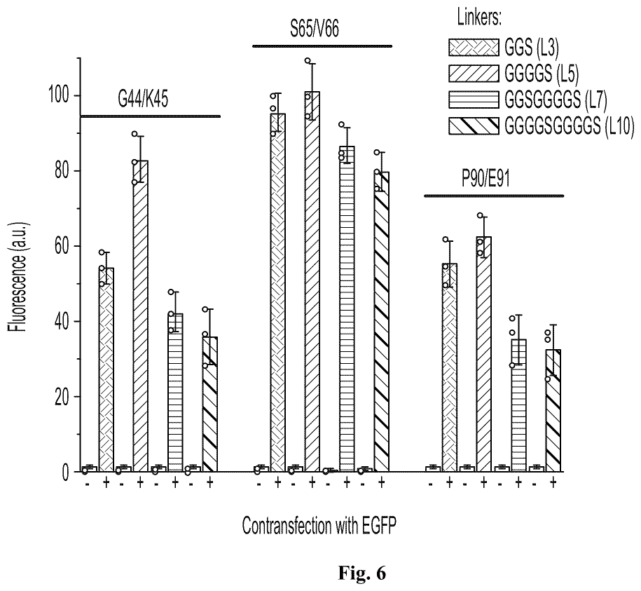

In cells coexpressing enhanced (EGFP), all internal fusions exhibited NIR fluorescence, which varied depending on the insertion site and linkers length. In the absence of EGFP, fluorescence of internal fusions was not detected (FIGS. 5C, 5D and 6), indicating that the fluorescence of fusions depended on the presence of a cognate antigen EGFP. In the brightest internal fusion protein, miRFP670nano3 was inserted between Nb_{GFP} residues Ser65 and Val66 using GGGGS linkers (FIGS. 5C, 5D and 6). This fusion variant was termed NIR-Fb_{GFP} and subjected to further analysis.

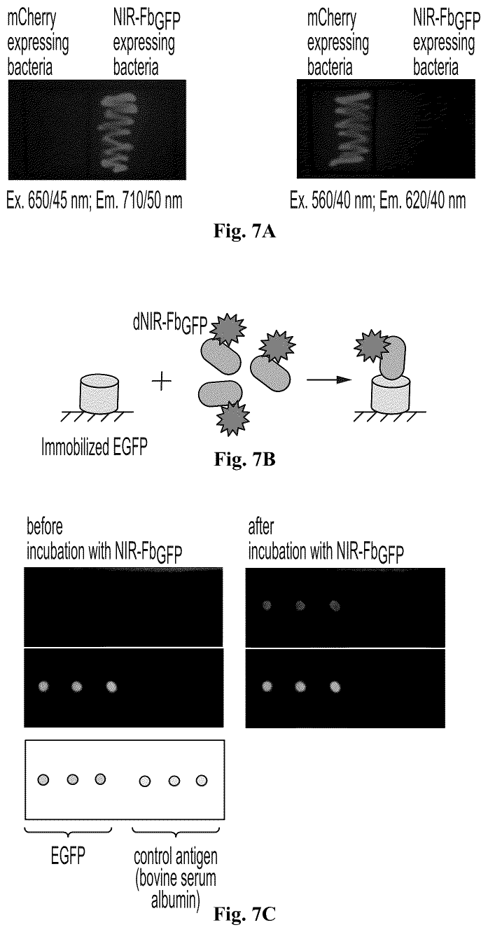

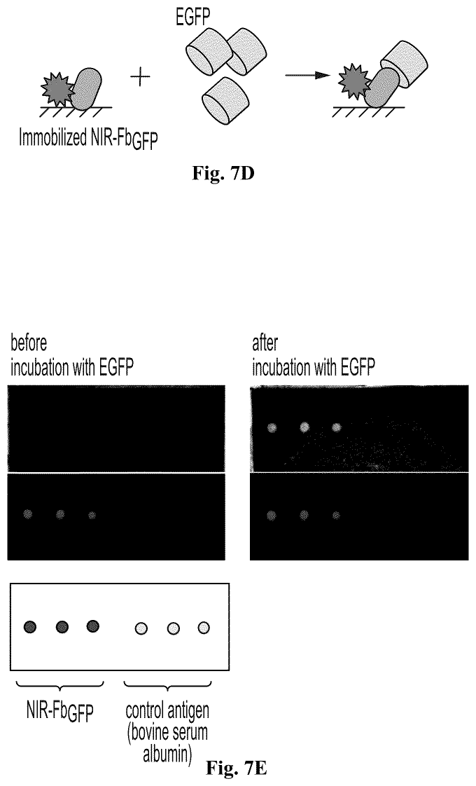

NIR-Fb_{GFP} was expressed in E, coli. In contrast to mammalian cells, bacterial cells expressing NIR-Fb without antigen exhibited NIR fluorescence (FIG. 7A). NIR-Fb_{GFP} purified from E, coli recognized immobilized EGFP and bound it from solution in the dot-blot analysis (FIGS. 7B-E), suggesting that NIR-Fb_{GFP} expressed in E, coli retained both the miRFP670nano3 fluorescence and Nb_{GFP} antigen-binding properties.

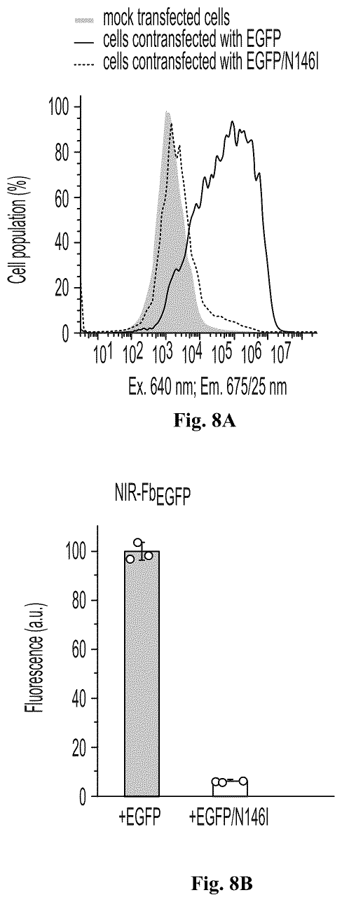

To determine whether a specificity to antigen affected NIR-Fb behavior, an EGFP variant bearing an Asn146Ile mutation was used. This mutation dramatically reduces binding to Nb_{GFP}. Coexpression of NIR-Fb_{GFP} and EGFP/Asn146Ile caused a >10-fold decrease in NIR fluorescence of HeLa cells as compared to the cells co-expressed NIR-Fb_{GFP} and non-mutated EGFP (FIGS. 8A and 8B), indicating that NIR-Fb_{GFP} performance depended on antigen specificity.

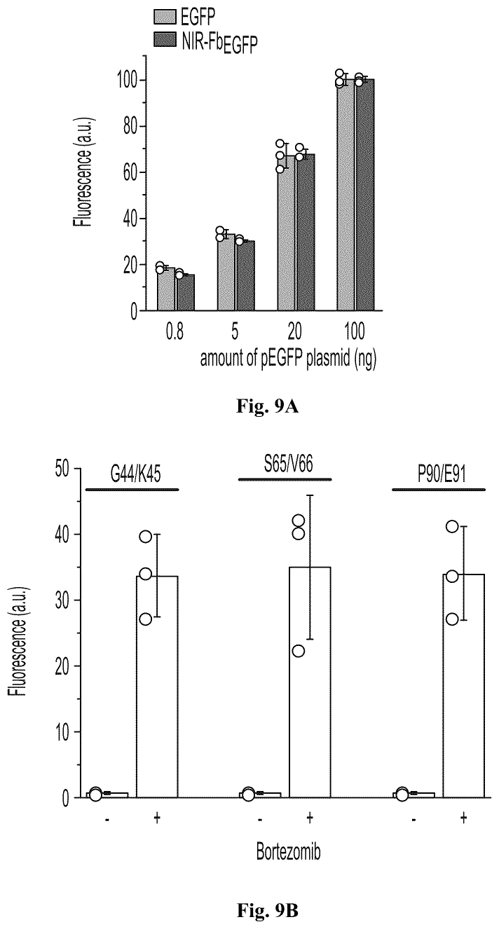

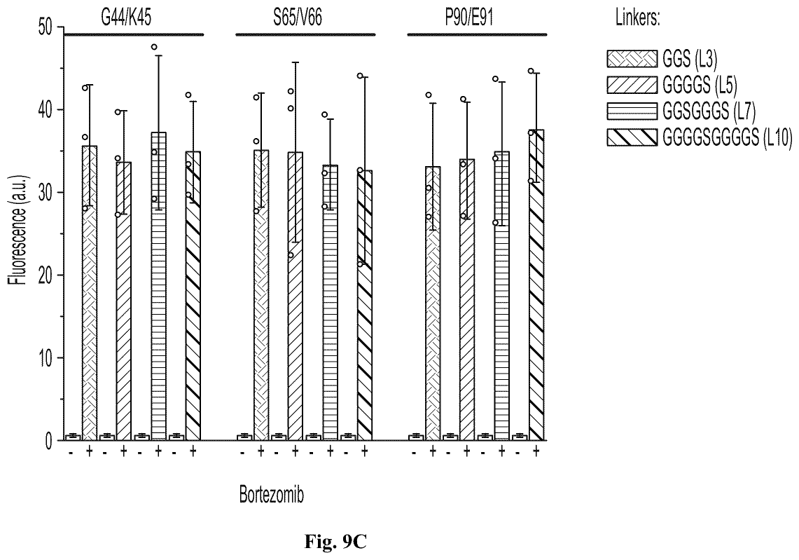

To assess whether an antigen concentration affected the protein level of NIR-Fb, the same amount of NIR-Fb_{GFP} plasmid was co-transfected with different amounts of EGFP plasmid. Fluorescence of NIR-Fb_{GFP} strongly depended on EGFP expression level (FIG. 9A), confirming that NIR-Fb exhibits antigen-dependence in a dose-dependent manner.

The data indicated that a possible mechanism of NIR-Fb_{GFP} behavior in mammalian cells is the stabilization by antigen binding and degradation in the unbound state by the ubiquitin-proteasome system. To further evaluate this hypothesis, cells transfected with NIR-Fb_{GFP} were treated with bortezomib, an inhibitor of proteasome-dependent protein degradation. Whereas the non-treated cells were non-fluorescent, cells treated with bortezomib demonstrated strong NIR fluorescence (FIGS. 9B and 9C). Taken together, these data indicated that in mammalian cells, the presence of the specific antigen stabilizes NIR-Fbs, whereas in the unbound state, NIR-Fbs degrade.

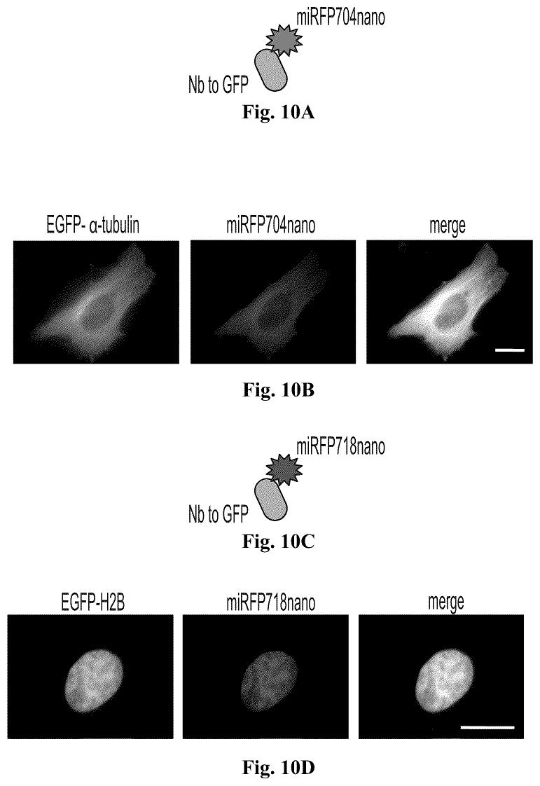

Similarly, NIR-Fb_{GFP} constructs comprising miRFP704nano (FIGS. 10A and 10B) or miRFP718nano (FIGS. 10C and 10D) instead of miRFP670nano3 efficiently recognized EGFP-fusions.

### Example 8: Engineering of NIR-Fbs Using Different Nanobodies

To study whether the NIR-Fb approach can be extended to other Nbs, the approach was applied to nine more Nbs, such as Nb specific to actin, BC2 Nb to β-catenin, LaM4 Nb to mCherry, Nb against ALFA-tag, Nb ca1698 to E, coli DHFR (dihydrofolate reductase), Nb 59H10 and 2E7 to HIV (human immunodeficiency virus) antigens p2436 and gp41, and Nb 21 and m6 specific to spike SARS-Cov-2 antigen (FIG. 11).

Although these Nbs have similar structural architecture, their mode of binding to antigen is different. Nb_{GFP} employs for antigen binding all three CDR regions (PDB ID: 3OGO). In contrast, in the LaM4 interaction with mCherry, the binding occurs via the CDR3 and only partly via CDR1 (PDB ID: 6IR1). Nb m6 binding to the spike SARS-COV-2 RBD (receptor binding domain) is mediated by the CDR1 and CDR2. The interactions in the BC2 Nb are formed by the CDR3 and framework regions 2 and 3 (PDB ID: 5IVN). Nb_{ALFA} binding of the ALFA-peptide is mainly mediated by CDRs 2 and 3 but also involves framework regions 2 (PDB ID: 6I2G). Nb ca1698 binds to DHFR using residues from all three CDRs and framework 2 (PDB ID: 4EIG). To interact with the C-terminal domain of the HIV p24 antigen, Nb 59H10 uses its framework region 2 and all three CDRs (PDB ID: 5O2U)36. The major contacts of the Nb 2E7 and HIV gp41 antigen are formed by residues in CDR1, CDR2 and framework region 2 (PDB ID:5HM1). Nb 21 binds to spike SARS-COV-2 RBD via all three CDRs and framework regions 2 and 339.

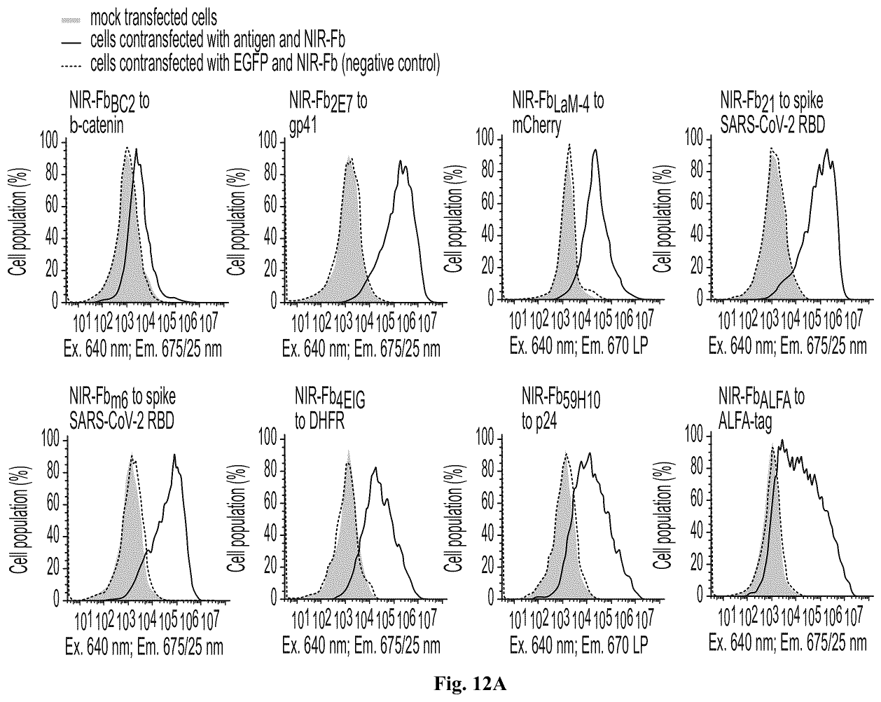

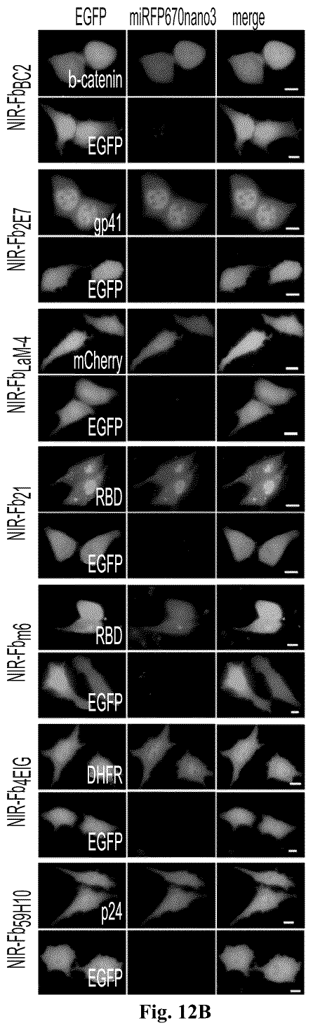

Despite the differences in the structural basis of antigen binding, all engineered NIR-Fbs exhibited antigen-dependent stabilization in mammalian cells, similar to NIR-Fb_{GFP}. The eight engineered NIR-Fbs exhibited strong fluorescence in cells co-expressing their cognate antigens and degraded in the absence of specific antigens (FIGS. 12A and 12B). Only NIR-FbBC2 produced rather weak fluorescence that was consistent with properties of the BC2 Nbs, which were reported to have low intracellular expression.

### Example 9: Re-Coloring of Fluorescent Proteins Using NIR-Fbs

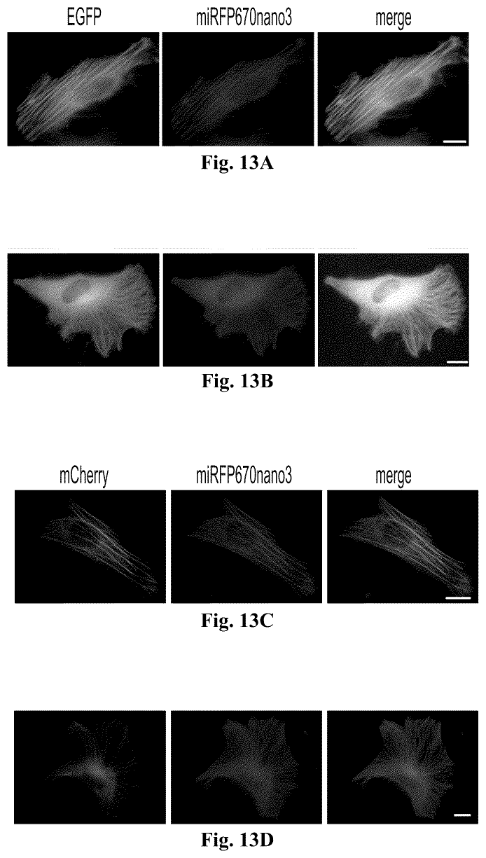

To date, many transgenic mice and cell lines expressing proteins of interest labeled with EGFP or mCherry are available. Accordingly, the NIR-Fb technology was next used for re-coloring of EGFP- and mCherry-labeled proteins. HeLa cells were co-transfected with EGFP- or mCherry-tagged β-actin and α-tubulin and a 10-fold excess of NIR-Fb_{GFP} and NIR-Fb_{mCherry} encoding plasmids. Despite this, both NIR-Fb_{GFB} and NIR-Fb_{mCherry} strongly colocalized with the EGFP- or mCherry-labeled cytoskeletal proteins, and no background signal was observed (FIGS. 13A-D).

The NIR re-coloring with NIR-Fb_{GFP} and NIR-Fb_{mCherry} can be applied to numerous available transgenic mice and cell lines expressing proteins of interest tagged with EGFP or mCherry. NIR-Fbs exhibit antigen-dependence stabilization in the dose-dependent manner (FIG. 9A), which allows the re-coloring of cells heterogenic by the EGFP or mCherry expression level. Additional NIR colors can be used by swapping miRFP670nano3 with miRFP704nano or miRFP718nano in NIR-Fbs (FIGS. 10A and 10B).

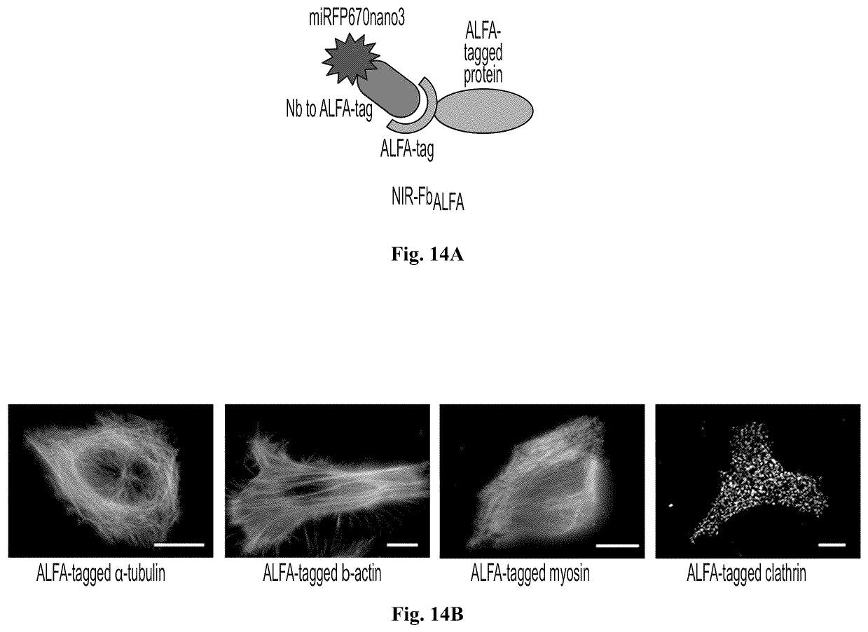

Notably, NIR-Fb_{ALFA} engineered with Nb specific to ALFA tag performed well in the labeling of various ALFA-tagged cellular proteins (FIGS. 14A and 14B).

### Example 10: Fusions of NIR-Fbs with Other Fluorescent Proteins for Indirect Labeling of Target Proteins

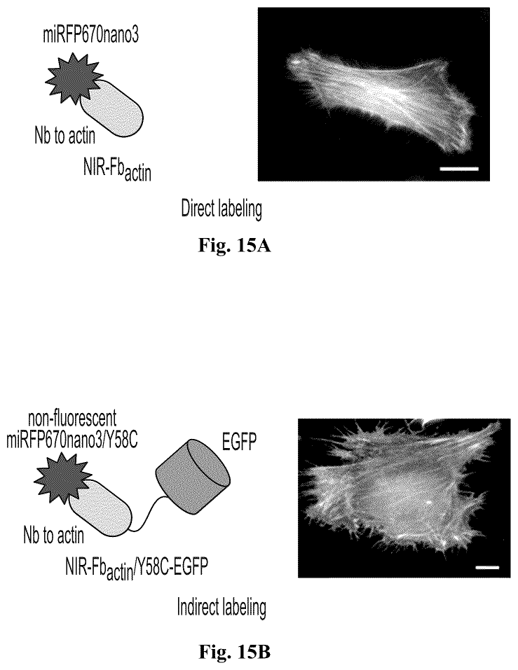

Next, it was demonstrated that NIR-Fbs can be used for the indirect labeling of a protein of interest (e.g., actin), by fusion a non-fluorescent miRFPnano variant with another fluorescent protein (e.g., EGFP). To avoid double-labeling, miRFP670nano3/Tyr58Cys was generated to serve as a non-fluorescent miRFPnano variant (FIGS. 15A and 15B).

### Example 11: Bispecific NIR-Fbs for Labeling of Double-Positive Cells

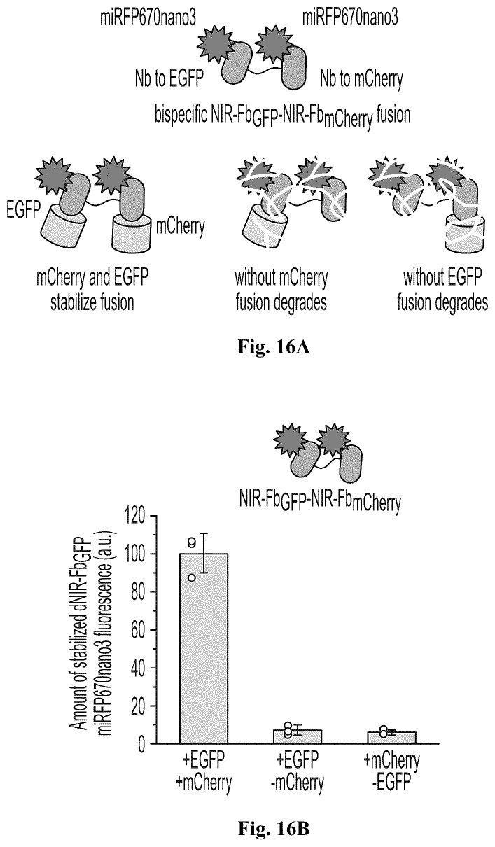

Next, a bispecific fusion construct containing NIR-Fbs to two different cognate antigens, EGFP and mCherry, was generated (FIG. 16A).

Co-expression of both EGFP and mCherry resulted in miRFP670nano3 fluorescence, indicating that the bispecific NIR-Fb_{GFP}-NIR-Fb_{mCherry} fusion was stabilized by binding with antigens. In contrast, when the bispecific NIR-Fb_{GFP}-NIR-Fb_{mCherry} fusion was coexpressed with either only EGFP or only mCherry, the NIR fluorescence dropped >10-fold, indicating that the bispecific fusion had been degraded (FIG. 16B).

Thus, bispecific NIR-Fbs permit specific labeling of double-positive cell populations expressing both targeted antigens, efficiently acting as Boolean “and” element. This property makes these constructs very useful for the design of new signaling pathways and other synthetic biology applications in mammalian cells.

### Example 12: NIR-Fbs for Antigen-Dependent Gene Regulation

Next, the suitability of NIR-Fb as destabilizing fusion-partner for regulatory proteins as determined.

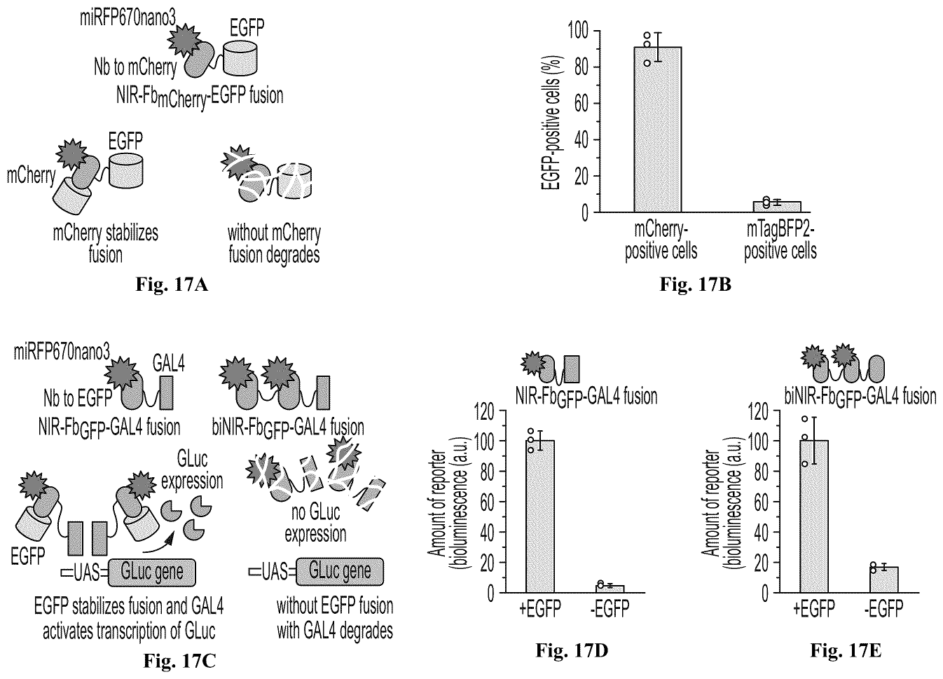

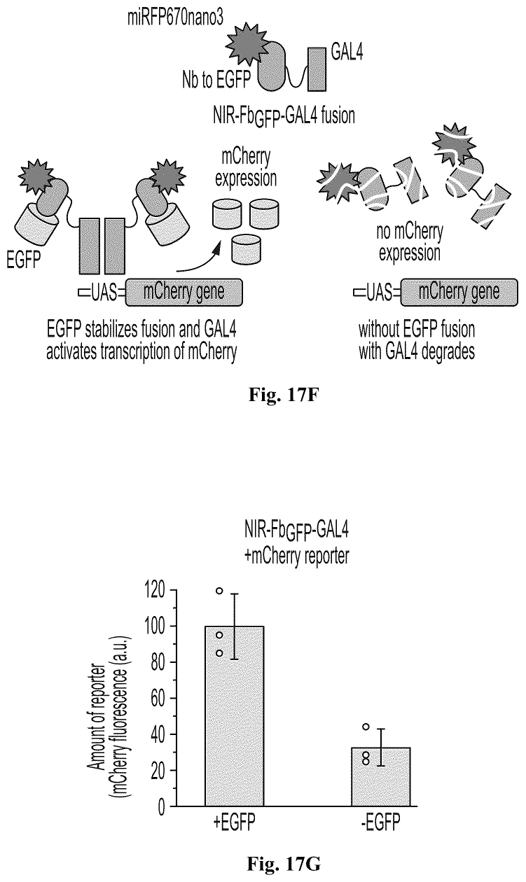

First, to confirm that NIR-Fb mediates antigen-dependent stability of its fusion-partner, a NIR-Fb_{mCherry}-EGFP fusion was generated and its behavior in live cells cotransfected with mCherry or mTagBFP2, respectively, was evaluated (FIGS. 17A and 17B). EGFP fluorescence was observed only in the cells expressing mCherry (FIGS. 17A and 17B), indicating that in the absence of cognate antigen, both NIR-Fb_{mCherry} and its fusion-partner were EGFP degraded.

Then, this approach was applied to antigen-dependent gene expression. For this, a GAL4/UAS system was used in which the GAL4 transcription factor drives expression of the reporter gene downstream of its UAS (upstream activating sequence)'s DNA-binding site. GAL4 was fused to NIR-Fb_{GFP} (FIG. 17C, left) and co-expressed it with the pUAS-Gluc reporter plasmid encoding Gaussia luciferase (Gluc) and with either EGFP or mTagBFP2. When mTagBFP2 was co-expressed instead of EGFP, the NIR-Fb_{GFP}-GAL4 degraded, resulting in the 25-fold decrease of the bioluminescent signal as compared to cells co-transfected with EGFP (FIG. 17D). A biNIR-Fb_{GFP}-GAL4 construct containing two NIR-Fb_{GFP} fused with GAL4 (FIG. 17C, right) exhibited even stronger antigen-dependence, providing more than the 50-fold decrease of the Gluc signal with co-expressed mTagBFP2 as compared to the EGFP-expressing cells (FIG. 17E).

In a related experiment, GAL4 was fused with NIR-Fb_{GFP} (FIG. 17F) and coexpressed with a pUAS-mCherry reporter and either EGFP or mTagBFP, respectively. In the presence of EGFP, transfection with NIR-Fb_{GFP}-GAL4 resulted in efficient mCherry expression (FIG. 17G, left bar). In contrast, in the absence of EGFP, the NIR-Fb_{GFP}-GAL4 construct was degraded, resulting in a 3-fold reduction of mCherry level (FIG. 17G, right bar).

In sum, these data showed that cellular proteins can be targeted for the directed degradation in an antigen-dependent manner by replacing the Nb directed against GFP in the NIR-Fb_{mCherry}-Nb_{GFP} fusion with a Nb directed against the protein of interest.

### Example 13: NIR-Fbs for Targeted Degradation of Cellular Proteins

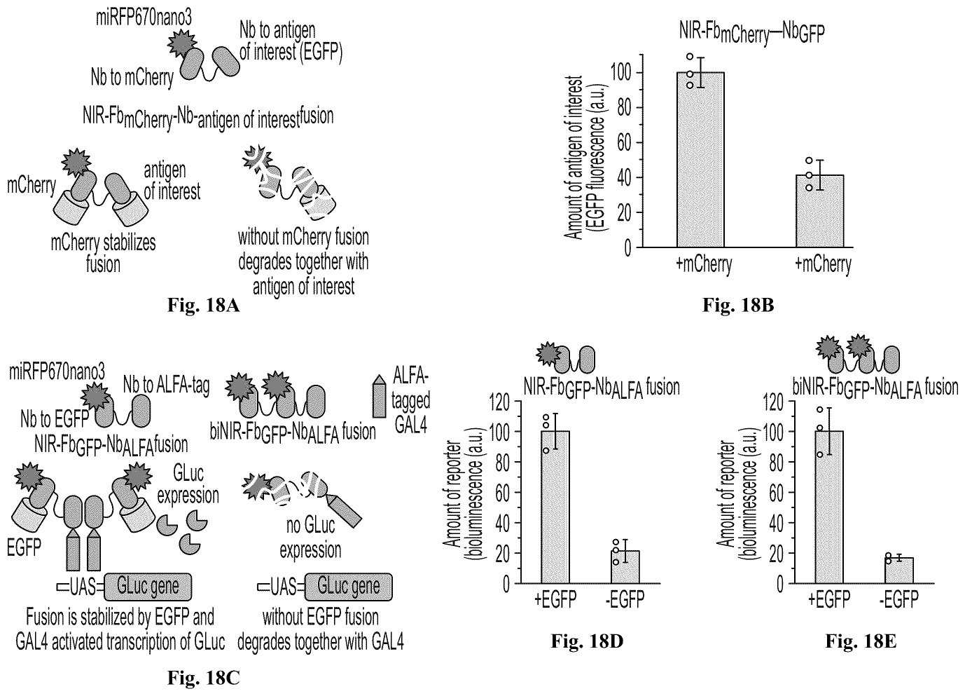

Next, it was demonstrated that NIR-Fbs can be used as a destabilizing component for proteolysis of a targeted protein, such as EGFP. To demonstrate this, a fusion of NIR-Fb_{mCherry} and Nb_{GFP} was constructed (FIG. 18A). The resulting NIR-Fb_{mCherry}-Nb_{GFP} fusion and EGFP were coexpressed in Hela cells with the same amounts of either mCherry or mTagBFP (a monomeric blue fluorescent protein). Expression of mCherry and mTagBFP was assessed in live cells using fluorescence microscopy. High fluorescence of both EGFP and miRFP670nano3 was observed in the presence of mCherry, which was the specific NIR-Fb_{mCherry} antigen (FIG. 18B, left bar). In the absence of mCherry, miRFP670nano3 fluorescence was not detected, and a ˜60% reduction in EGFP fluorescence was observed, indicating that EGFP degraded together with the NIR-Fb_{mCherry}-Nb_{GFP} fusion (FIG. 18B, right bar).

In a related experiment, Nb_{ALFA} and its small antigen ALFA peptide (SRLEEELRRRLTE, SEQ ID NO:38) fused to a protein were used. The degradation of the GAL4 transcription factor was tested by fusing NIR-Fb_{GFP} and biNIR-Fb_{GFP} with Nb_{ALFA} and GAL4 with ALFA peptide (FIG. 18C). Co-expression of ALFA-labeled GAL4 and pUAS-Gluc with NIR-Fb_{GFP}-Nb_{ALFA} without EGFP resulted in ˜75% reduction of the Gluc signal as compared to the EGFP-expressing cells (FIG. 18D). Moreover, with biNIR-Fb_{GFP}-Nb_{ALFA} the ˜85% reduction of the Gluc signal was observed in the absence of EGFP (FIG. 18E).

These data show that intracellular proteins can be targeted for the directed antigen-dependent degradation mediated by binding with NIR-Fb fusions.

### Example 14: NIR-Fbs for Control of Kinase Activity in an Antigen-Dependent Manner

Next, NIR-Fb was generated for the modulation of protein kinase activity.

Protein Kinase A (PKA) and c-Jun N-terminal kinase (JNK) were selected. PKA is one of the key effectors of the CAMP-dependent pathway, while JNK regulates cellular responses to stress signals. The activity of PKA could be specifically inhibited by a peptide comprising amino acids 14-22 (GRTGRRNAI, SEQ ID NO:36) of PKA inhibitor protein PKI. This peptide binds to the catalytic subunit of PKA and acts as pseudosubstrate. For inhibition of JNK activity, the widely used inhibitory peptide composed of the amino acids 153-163 (RPKRPTTLNLF, SEQ ID NO:40) of JNK-interacting protein-1 JIP-1 was chosen. The binding of this peptide induces rearrangement between the N- and C-terminal domains of JNK and distorts the ATP-binding pocket, inhibiting the catalytic activity.

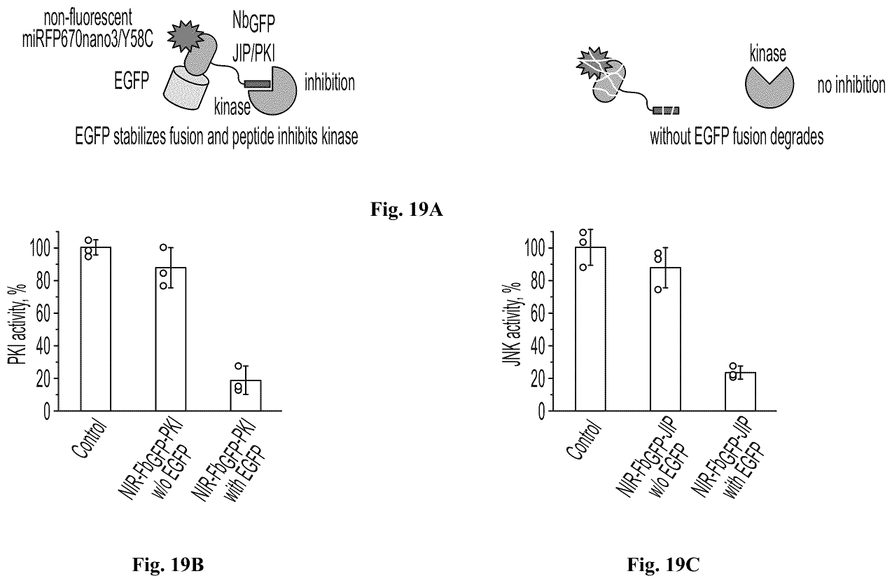

To develop antigen-dependent inhibitors for PKA and JNK kinases, constructs were engineered that consisted of corresponding inhibitory peptides and the non-fluorescent NIR-Fb_{GFP}/Tyr58Cys variant (FIG. 19A). The resultant NIR-Fb_{GFP}/Tyr58Cys-PKI and NIR-Fb_{GFP}/Tyr58Cys-JIP fusions were expressed in the presence and absence of EGFP in HeLa cell lines that produced NIR FRET biosensors of PKA or JNK kinases, respectively. Kinase activity was determined with the NIR FRET biosensors after stimulation of PKA with 1 mM dibutyryl cyclic adenosine monophosphate (dbcAMP) and JNK with 1 μg/ml anisomycin.

Co-transfection with NIR-Fb_{GFP}/Y58C-PKI (FIG. 19B) and NIR-Fb_{GFP}/Y58C-JIP (FIG. 19C) fusions with EGFP resulted in the inhibition of PKA and JNK kinases by ˜80% and ˜76%, respectively. In contrast, no effects on kinases activation were observed without the EGFP antigen (FIGS. 19B and 19C).

These results demonstrate the suitability of NIR-Fb inhibitory fusions to modulate kinases signaling events in an antigen-dependent manner.

### Example 15: NIR-Fbs for Multiplex Two-Photon Imaging in the Brain and Spinal Cord In Vivo

To evaluate the utility of miRFP670nano3 for in vivo imaging, the miRFPnano variant was subcloned into an adeno-associated viral (AAV) vector carrying a synapsin promoter. The vector was then injected into the somatosensory cortex or spinal dorsal horn of Cx3cr1^{GFP/+} mice with labeled microglia. Two to five weeks after injection, animals were prepared for in vivo two-photon imaging to determine protein expression and attainable imaging depth. NIR FPs in addition to their absorption peaks in the so-called Q band (i.e., 30-70 GM at 1200-1280 nm), exhibit high cross-sectional values in the so-called Soret band (180-450 GM at 890-950 nm). This feature permits multiplex, subcellular resolution imaging of miRFP670nano3 and EGFP with single-wavelength two-photon excitation using a standard Ti:Sapphire laser. The experiments used excitation light powers that, do not activate microglia, which are highly sensitive to perturbations in tissue physiology.

miRFP670nano3 brightly fluoresced in transduced cells without the need for external BV. At the 920 nm wavelength optimally exciting EGFP, miRFP670nano3-positive cells were routinely visualized through the entire depth of the cortex, down to the entorhinal cortex level (˜850 μm) that exceeded those with EGFP in Cx3cr1^{GFP/+} mice with brightly EGFP-labeled microglia. No photobleaching or cytotoxicity was observed.

It has been demonstrated that microglia respond to neuronal tissue perturbations with morphological changes and functional alterations. Such changes were not observed in response to the long-term (3-5 weeks) miRFP670nano3 expression (data not shown), demonstrating that miRFPnanos do not cause cytotoxicity and are well tolerated in vivo.

### Example 16: Utility of NIR-Fb for Targeted Intracellular Antigen Dependent Killing of Cells

Immunotoxins are chimeric proteins containing an antibody or other antigen binding molecules attached to a toxin. Typically, antigen-binding components are specific to surface proteins of cancer cells and mediated delivery of toxic agents, which killed targeted cells. Therefore, targeted molecules of classical immunotoxins are limited to those expressed on the cell surface. In contrast, the antigen-dependent properties of NIR-Fbs' fusions allow their use as intracellular immunotoxins, specific to targeted molecules present inside cells.

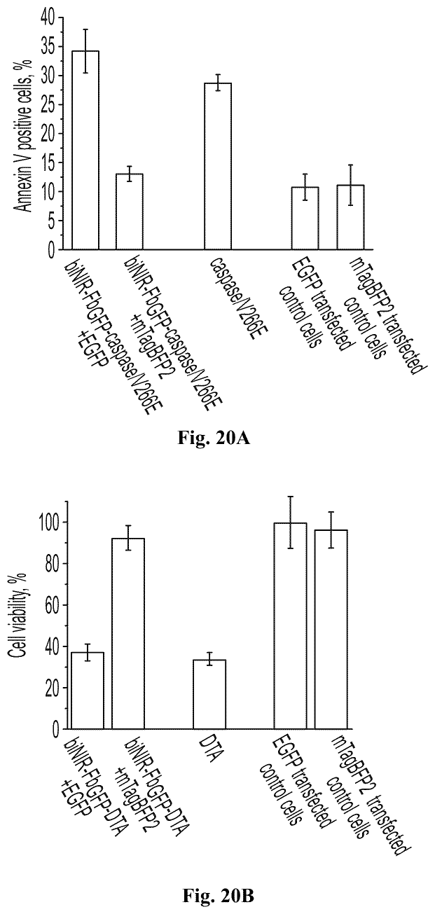

To test the utility of NIR-Fb for targeted intracellular antigen dependent killing of cells, a fusion of biNIR-Fb_{GFP} and constitutively active caspase-3 mutant V266E, which induces apoptosis, was constructed. The biNIR-Fb_{GFP} caspase/V266E fusion was expressed in the presence and absence of EGFP and cell viability was monitored by Annexin V staining. Co-transfection with biNIR-Fb_{GFP}-caspase/V266E and with EGFP resulted in cell death, similar to transfection with caspase/V266E. When mTagBFP2 was co-expressed with biNIR-Fb_{GFP}-caspase/V266E instead of EGFP, observed apoptosis level was close to that observed for control cells (FIG. 20A).

Next, a fusion of biNIR-Fb_{GFP} and diphtheria toxin A subunit (DTA), one of the most frequently used toxic component for immunotoxin development, was constructed. Co-transfection with biNIR-Fb_{GFP}-DTA and with EGFP resulted in robust cell death, similar to observed under cell transfection with DTA. Instead, the viability of cells transfected with biNIR-Fb_{GFP}-DTA, but without EGFP was close to control cells (FIG. 20B). These data demonstrate that NIR-Fb-based immunotoxins can be targeted to intracellular antigens, in contrast to classical immunotoxins targeted against surface proteins.

### Example 17: NIR-Fbs for Secretion of Intracellular Proteins

Next, it was determined whether NIR-Fbs can mediate specific transport of intracellular molecules for their further detection or presentation on cell membrane. For this, biNIR-Fb_{GFP} was fused to Gluc, containing natural secretion signal peptide (MGVKVLFALICIAVAE, SEQ ID NO:35). Then, cells were co-transfected with Gluc-biNIR-Fb_{GFP} and with either EGFP or mTagBFP2. Both, EGFP and mTagBFP2 did not contain any secretion signals.

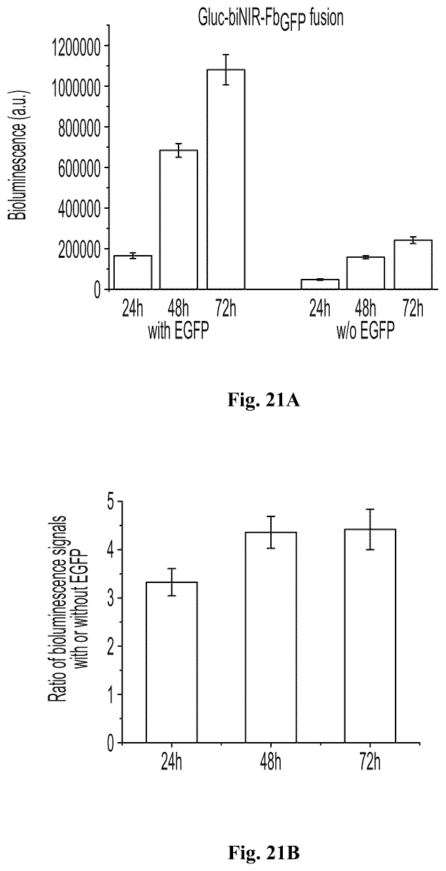

Co-expression of Gluc-biNIR-Fb_{GFP} fusion with EGFP resulted in robust Gluc signal (FIG. 21A). When Gluc-biNIR-Fb_{GFP} fusion was co-expression with mTagBFP2 instead of EGFP ˜78% reduction of the Gluc signal was observed (FIGS. 21A and 21B). These data demonstrated that NIR-Fb fusions containing secretion signals are stabilize by antigen binding and degrade in unbound state, similar to NIR-Fb expressed as cytosolic proteins.

### Example 18: Sequences for Exemplary Constructs with GGGGS-Linkers

In bold are sequences of N- and C-terminal helices of miRFP670nano3. The sequence of miRFP670nano3 is underlined.

miRFP670nano3 inserted at G44/K45 site of
nanobody to GFP (Nb_{GFP})
(SEQ ID NO: 18)
MDQVQLVESGGALVQPGGSLRLSCAASGFPVNRYSMRWYRQAPGGGGGS
MANLDKMLNTTVTEVRKF
LQADRVCVFKFEEDYSGTVSHEAVDDRWISILKTQVQDRYFMETRGEEY
VHGRYQAIADIYTANLVECYRDLLIEFQVRAILAVPILQGKKLWGLLVA
HQLAGPREWQ
TWEIDFLKQQAVVMGIAIQQS
GGGGSKEREWVAGMSSAGDRSSYEDSVKGRFTISRDDARNTVYLQMNSLK
PEDTAVYYCNVNVGFEYWGQGTQVTVSS
miRFP670nano3 inserted at S65/V66 site of
nanobody to GFP (Nb_{GFP})
(SEQ ID NO: 19)
MDQVQLVESGGALVQPGGSLRLSCAASGFPVNRYSMRWYRQAPGKEREWV
AGMSSAGDRSSYEDSGGGGS
MANLDKMLNTTVTEVRKF
LQADRVCVFKFEEDYSGTVSHEAVDDRWISILKTQVQDRYFMETRGEEYV
HGRYQAIADIYTANLVECYRDLLIEFQVRAILAVPILQGKKLWGLLVAHQ
LAGPREWQ
TWEIDFLKQQAVVMGIAIQQS
GGGGSVKGRFTISRDDARNTVYLQMNSLKPEDTAVYYCNVNVGFEYWGQG
TQVTVSS
miRFP670nano3 inserted at P90/E91 site of
nanobody to GFP (Nb_{GFP}). The most destabilized
NIR-Fb variant
(SEQ ID NO: 20)
MDQVQLVESGGALVQPGGSLRLSCAASGFPVNRYSMRWYRQAPGKEREWV
AGMSSAGDRSSYEDSVKGRFTISRDDARNTVYLQMNSLKPGGGGS
MANLDKMLNTTVTEVRKF
LQADRVCVFKFEEDYSGTVSHEAVDDRWISILKTQVQDRYFMETRGEEYV
HGRYQAIADIYTANLVECYRDLLIEFQVRAILAVPILQGKKLWGLLVAHQ
LAGPREWQ
TWEIDFLKQQAVVMGIAIQQS
GGGGSEDTAVYYCNVNVGFEYWGQGTQVTVSS

### Example 19: Sequences for Exemplary Constructs with GGS-Linkers

miRFP670nano3 inserted at G44/K45 site of
nanobody to GFP (Nb_{GFP})
(SEQ ID NO: 21)
MDQVQLVESGGALVQPGGSLRLSCAASGFPVNRYSMRWYRQAPGGGSMAN
LDKMLNTTVTEVRKFLQADRVCVFKFEEDYSGTVSHEAVDDRWISILKTQ
VQDRYFMETRGEEYVHGRYQAIADIYTANLVECYRDLLIEFQVRAILAVP
ILQGKKLWGLLVAHQLAGPREWQTWEIDFLKQQAVVMGIAIQQSGGSKER
EWVAGMSSAGDRSSYEDSVKGRFTISRDDARNTVYLQMNSLKPEDTAVYY
CNVNVGFEYWGQGTQVTVSS
miRFP670nano3 inserted at S65/V66 site of
nanobody to GFP (Nb_{GFP})
(SEQ ID NO: 22)
MDQVQLVESGGALVQPGGSLRLSCAASGFPVNRYSMRWYRQAPGKEREWV
AGMSSAGDRSSYEDSGGSMANLDKMLNTTVTEVRKFLQADRVCVFKFEED
YSGTVSHEAVDDRWISILKTQVQDRYFMETRGEEYVHGRYQAIADIYTAN
LVECYRDLLIEFQVRAILAVPILQGKKLWGLLVAHQLAGPREWQTWEIDF
LKQQAVVMGIAIQQSGGSVKGRFTISRDDARNTVYLQMNSLKPEDTAVYY
CNVNVGFEYWGQGTQVTVSS
miRFP670nano3 inserted at P90/E91 site of
nanobody to GFP (Nb_{GFP})
(SEQ ID NO: 23)
MDQVQLVESGGALVQPGGSLRLSCAASGFPVNRYSMRWYRQAPGKEREWV
AGMSSAGDRSSYEDSVKGRFTISRDDARNTVYLQMNSLKPGGSMANLDKM
LNTTVTEVRKFLQADRVCVFKFEEDYSGTVSHEAVDDRWISILKTQVQDR
YFMETRGEEYVHGRYQAIADIYTANLVECYRDLLIEFQVRAILAVPILQG
KKLWGLLVAHQLAGPREWQTWEIDFLKQQAVVMGIAIQQSGGSEDTAVYY
CNVNVGFEYWGQGTQVTVSS

### Example 20: Sequences for Exemplary Constructs with GGSGGGS-Linkers

miRFP670nano3 inserted at G44/K45 site of
nanobody to GFP (Nb_{GFP})
(SEQ ID NO: 24)
MDQVQLVESGGALVQPGGSLRLSCAASGFPVNRYSMRWYRQAPGGGSGGG
SMANLDKMLNTTVTEVRKFLQADRVCVFKFEEDYSGTVSHEAVDDRWISI
LKTQVQDRYFMETRGEEYVHGRYQAIADIYTANLVECYRDLLIEFQVRAI
LAVPILQGKKLWGLLVAHQLAGPREWQTWEIDFLKQQAVVMGIAIQQSGG
SGGGSKEREWVAGMSSAGDRSSYEDSVKGRFTISRDDARNTVYLQMNSLK
PEDTAVYYCNVNVGFEYWGQGTQVTVSS
miRFP670nano3 inserted at S65/V66 site of
nanobody to GFP (Nb_{GFP})
(SEQ ID NO: 25)
MDQVQLVESGGALVQPGGSLRLSCAASGFPVNRYSMRWYRQAPGKEREWV
AGMSSAGDRSSYEDSGGSGGGSMANLDKMLNTTVTEVRKFLQADRVCVFK
FEEDYSGTVSHEAVDDRWISILKTQVQDRYFMETRGEEYVHGRYQAIADI
YTANLVECYRDLLIEFQVRAILAVPILQGKKLWGLLVAHQLAGPREWQTW
EIDFLKQQAVVMGIAIQQSGGSGGGSVKGRFTISRDDARNTVYLQMNSLK
PEDTAVYYCNVNVGFEYWGQGTQVTVSS
miRFP670nano3 inserted at P90/E91 site of
nanobody to GFP (Nb_{GFP})
(SEQ ID NO: 26)
MDQVQLVESGGALVQPGGSLRLSCAASGFPVNRYSMRWYRQAPGKEREWV
AGMSSAGDRSSYEDSVKGRFTISRDDARNTVYLQMNSLKPGGSGGGSMAN
LDKMLNTTVTEVRKFLQADRVCVFKFEEDYSGTVSHEAVDDRWISILKTQ
VQDRYFMETRGEEYVHGRYQAIADIYTANLVECYRDLLIEFQVRAILAVP
ILQGKKLWGLLVAHQLAGPREWQTWEIDFLKQQAVVMGIAIQQSGGSGGG
SEDTAVYYCNVNVGFEYWGQGTQVTVSS

### Example 21: Sequences for Exemplary Constructs with GGGGSGGGGS-Linkers

miRFP670nano3 inserted at G44/K45 site of
nanobody to GFP (Nb_{GFP})
(SEQ ID NO: 27)
MDQVQLVESGGALVQPGGSLRLSCAASGFPVNRYSMRWYRQAPGGGGGSG
GGGSMANLDKMLNTTVTEVRKFLQADRVCVFKFEEDYSGTVSHEAVDDRW
ISILKTQVQDRYFMETRGEEYVHGRYQAIADIYTANLVECYRDLLIEFQV
RAILAVPILQGKKLWGLLVAHQLAGPREWQTWEIDFLKQQAVVMGIAIQQ
SGGGGSGGGGSKEREWVAGMSSAGDRSSYEDSVKGRFTISRDDARNTVYL
QMNSLKPEDTAVYYCNVNVGFEYWGQGTQVTVSS
miRFP670nano3 inserted at S65/V66 site of
nanobody to GFP (Nb_{GFP})
(SEQ ID NO: 28)
MDQVQLVESGGALVQPGGSLRLSCAASGFPVNRYSMRWYRQAPGKEREWV
AGMSSAGDRSSYEDSGGGGSGGGGSMANLDKMLNTTVTEVRKFLQADRVC
VFKFEEDYSGTVSHEAVDDRWISILKTQVQDRYFMETRGEEYVHGRYQAI
ADIYTANLVECYRDLLIEFQVRAILAVPILQGKKLWGLLVAHQLAGPREW
QTWEIDFLKQQAVVMGIAIQQSGGGGSGGGGSVKGRFTISRDDARNTVYL
QMNSLKPEDTAVYYCNVNVGFEYWGQGTQVTVSS
miRFP670nano3 inserted at P90/E91 site of
nanobody to GFP (Nb_{GFP})
(SEQ ID NO: 29)
MDQVQLVESGGALVQPGGSLRLSCAASGFPVNRYSMRWYRQAPGKEREWV
AGMSSAGDRSSYEDSVKGRFTISRDDARNTVYLQMNSLKPGGGGSGGGGS
MANLDKMLNTTVTEVRKFLQADRVCVFKFEEDYSGTVSHEAVDDRWISIL
KTQVQDRYFMETRGEEYVHGRYQAIADIYTANLVECYRDLLIEFQVRAIL
AVPILQGKKLWGLLVAHQLAGPREWQTWEIDFLKQQAVVMGIAIQQSGGG
GSGGGGSEDTAVYYCNVNVGFEYWGQGTQVTVSS

TABLE 3
Summary of additional amino acid sequences

| SEQ | | |
|---|---|---|
| ID | Descrip- | |
| NO | tion | Sequence |
| 8 | Nb_{GFP} | QVQLVESGGALVQPGGSLRLSCAASGFPVNRY |
| | | SMRWYRQAPGKEREWVAGMSSAGDRSSYEDSV |
| | | KGRFTISRDDARNTVYLQMNSLKPEDTAVYYC |
| | | NVNVGFEYWGQGTQVTVSS |
| 9 | Nb_{mCherry} | QLVESGGSLVQPGGSLRLSCAASGRFAESSSM |
| | | GWFRQAPGKEREFVAAISWSGGATNYADSAKG |
| | | RFTLSRDNTKNTVYLQMNSLKPDDTAVYYCAA |
| | | NLGNYISSNQRLYGYWGQGTQVTVS |
| 10 | Nb_{actin} | MAQVQLVESGGGLTQAGGSLRLSCATSGLIFS |
| | | AFGMGWFRQAPGKEREFVGGINWRGSTNYGDF |
| | | VKGRFTISRDNAKNTVYLQMNNLKPEDTAVYY |
| | | CAARMVHKTEYDYWGEGTQVTVSS |
| 11 | Nb_{catenin} | MQVQLVESGGGLVQPGGSLTLSCTASGFTLDH |
| | | YDIGWFRQAPGKEREGVSCINNSDDDTYYADS |
| | | VKGRFTIFMNNAKDTVYLQMNSLKPEDTAIYY |
| | | CAEARGCKRGRYEYDFWGQGTQVTVSS |
| 12 | Nb_{ALFA-tag} | MVQLQESGGGLVQPGGSLRLSCTASGVTISAL |
| | | NAMAMGWYRQAPGERRVMVAAVSERGNAMYRE |
| | | SVQGRFTVTRDFTNKMVSLQMDNLKPEDTAVY |
| | | YCHVLEDRVDSFHDYWGQGTQVTVSS |
| 13 | Nb_{DXFR} | MQVQLQESGGGLVQAGGSLRLSCKASGIIFSV |
| | | YKMTWYRQAPGKERELVALITTNNNTMTVDSV |
| | | KGRFTISRDNVQNTVYLEMNNLKPEDTAVYYC |
| | | NANRGLAGPAYWGQGTQVTVSS |
| 14 | Nbₚ₂₄ | MDVQLQESGGGLVQAGGSLRLSCAASGSISRF |
| | | NAMGWWRQAPGKEREFVARIVKGFDPVLADSV |
| | | KGRFTISIDSAENTLALQMNRLKPEDTAVYYC |
| | | FAALDTAYWGQGTQVTVSS |
| 15 | Nb_{gp41} | MDVQLQESGGGLVQPGGSLRLSCAASGNIVSI |
| | | DAAGWFRQAPGKQREPVATILTGGATNYADSV |
| | | KGRFTISRDNAKNTVYLQMNSLKPEDTAVYYC |
| | | YAPMIYYGGRYSDYWGQGTQVTVSS |
| 16 | Nb2₁ₛₚᵢₖₑ | MQVQLVESGGGLVQAGGSLRLSCAVSGLGAHR |
| | | VGWFRRAPGKEREFVAAIGANGGNTNYLDSVK |
| | | GRFTISRDNAKNTIYLQMNSLKPQDTAVYYCA |
| | | ARDIETAEYTYWGQGTQVTVSS |
| 17 | Nbₘ₆ₛₚᵢₖₑ | MQVQLVESGGGLVQAGGSLRLSCAASGYIFGR |
| | | NAMGWYRQAPGKERELVAGITRRGSITYYADS |
| | | VKGRFTISRDNAKNTVYLQMNSLKPEDTAVYY |
| | | CAADPASPAYGDYWGQGTQVTVSS |
| 30 | Example | GGGGS |
| | flexible | |
| | linker | |
| 31 | Example | GGSGGGS |
| | flexible | |
| | linker | |
| 32 | Example | GGGGSGGGGS |
| | flexible | |
| | linker | |
| 33 | Example | MANLDKMLNTTVTEVRKF |
| | poly- | |
| | peptide | |
| | forming | |
| | an | |
| | alpha | |
| | helix | |
| 34 | Example | TWEIDFLKQQAVVMGIAIQQS |
| | poly- | |
| | peptide | |
| | forming | |
| | an | |
| | alpha | |
| | helix | |
| 35 | Example | MGVKVLFALICIAVAE |
| | signal | |
| | sequence | |
| 36 | Example | GRTGRRNAI |
| | kinase | |
| | inhibiting | |
| | peptide | |
| 37 | Example | RPKRPTTLNLF |
| | kinase | |
| | inhibiting | |
| | peptide | |
| 38 | ALFA | SRLEEELRRRLTE |
| | peptide | |
| 39 | amino | GRTGRRNAI |
| | acids | |
| | 14-22 | |
| | of PKA | |
| | inhibitor | |
| | protein | |
| | PKI35 | |
| 40 | the amino | RPKRPTTLNLF |
| | acids | |
| | 153-163 | |
| | of JNK- | |
| | interacting | |
| | protein-1 | |
| | JIP-137 | |
| 41 | Example | GGGGS GGGGS GGGGS GGGGS |
| | flexible | |
| | linker | |

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiment, method, and examples, but by all embodiments and methods within the scope and spirit of the disclosure.

1. (canceled)

2. An internal fusion protein comprising a nanobody, wherein a polypeptide is inserted into the nanobody, and wherein the internal fusion protein comprises:
(a) a flexible linker located between the polypeptide inserted into the nanobody and the N-terminal portion of the nanobody;
(b) a flexible linker located between the polypeptide inserted into the nanobody and the C-terminal portion of the nanobody; or
(c) (i) a flexible linker located between the polypeptide inserted into the nanobody and the N-terminal portion of the nanobody and (ii) a flexible linker located between the polypeptide inserted into the nanobody and the C-terminal portion of the nanobody.

3. The internal fusion protein of claim 2, wherein the internal fusion protein comprises:
(a) a first polypeptide forming an alpha helix located between the polypeptide inserted into the nanobody and the N-terminal portion of the nanobody; and
(b) a second polypeptide forming an alpha helix located between the polypeptide inserted into the nanobody and the C-terminal portion of the nanobody.

4. The internal fusion protein of claim 3, wherein:
(a) the first polypeptide forming an alpha helix comprises a sequence that is at least 90% identical to SEQ ID NO:33 and the second polypeptide forming an alpha helix comprises a sequence that is at least 90% identical to SEQ ID NO:34; or
(b) the first polypeptide forming an alpha helix comprises a sequence that is at least 90% identical to SEQ ID NO:34 and the second polypeptide forming an alpha helix comprises a sequence that is at least 90% identical to SEQ ID NO:33.

5. The internal fusion protein of claim 3, wherein:
(a) the first polypeptide forming an alpha helix comprises SEQ ID NO:33 and the second polypeptide forming an alpha helix comprises SEQ ID NO:34; or
(b) the first polypeptide forming an alpha helix comprises SEQ ID NO:34 and the second polypeptide forming an alpha helix comprises SEQ ID NO:33.

6. The internal fusion protein of claim 2, wherein the polypeptide is inserted into the nanobody at a position corresponding to the i) G44/K45, (ii) S65/V66, or (iii) P90/E91 insertion site in a nanobody of SEQ ID NO:8.

7. The internal fusion protein of claim 2, wherein the polypeptide inserted into the nanobody is a fluorescent protein, a drug, or a toxin.

8. The internal fusion protein of claim 2, wherein the polypeptide inserted into the nanobody is a non-fluorescent protein.

9. The internal fusion protein of claim 7, wherein the fluorescent protein inserted into the nanobody is a near-infrared fluorescent protein.

10. The internal fusion protein of claim 9, wherein the near-infrared fluorescent protein inserted into the nanobody comprises a sequence that is at least 90% identical to any one of SEQ ID NOs: 1-6.

11. The internal fusion protein of claim 10, wherein the near-infrared fluorescent protein inserted into the nanobody comprises any one of SEQ ID NOs: 1-6.

12. The internal fusion protein of claim 2, wherein the nanobody specifically binds to a fluorescent protein, a tumor antigen, or an intracellular protein.

13. The internal fusion protein of claim 2, wherein the internal fusion protein is fused to a transcription factor.

14. The internal fusion protein of claim 2, wherein the polypeptide inserted into the nanobody is a kinase inhibitor.

15. The internal fusion protein of claim 14, wherein the kinase inhibitor comprises the sequence GRTGRRNAI (SEQ ID NO:39) or RPKRPTTLNLF (SEQ ID NO:40).

16. A multi-modular fusion protein comprising:
(a) a first internal fusion protein of claim 2; and
(b) a second internal fusion protein of claim 2.

17. The multi-modular fusion protein of claim 16, wherein the nanobody of the first internal fusion protein binds to a first antigen and the nanobody of the second internal fusion protein binds to a second antigen, and wherein the first and the second antigen are different.

18. The multi-modular fusion protein of claim 17, wherein the second antigen is targeted for degradation.

19. The internal fusion protein of claim 2, wherein the internal fusion protein or the multi-modular fusion protein comprises a signal peptide.

20. A nucleic acid encoding the internal fusion protein of claim 2.

21. A vector comprising the nucleic acid of claim 20.

22. The vector of claim 21, wherein the vector is a viral vector.

23. An isolated cell comprising the nucleic acid of claim 20.

24. A method of recoloring a cell expressing a first fluorescent protein, the method comprising:
(a) expressing the internal fusion protein of claim 2 in the cell, wherein a second fluorescent protein is inserted into the nanobody and wherein the nanobody binds to the first fluorescent protein;
(b) detecting the fluorescence of the second fluorescent protein.

25. A method of detecting the simultaneous presence of a first and a second antigen in a cell, the method comprising:
(a) expressing in the cell a first internal fusion protein of claim 2 fused to a second internal fusion protein of claim 2; wherein the first internal fusion protein comprises (i) a first nanobody that binds to the first antigen and (ii) a first fluorescent protein inserted into the first nanobody; and wherein the second internal fusion protein comprises (i) a second nanobody that binds to the second antigen and optionally (ii) a second fluorescent protein inserted into the second nanobody; and
(b) detecting the fluorescence of the first and optionally the fluorescence of the second fluorescent protein; wherein presence of fluorescence of the first and optionally of the second fluorescent protein indicates simultaneous presence of the first and the second antigen in the cell; and absence of fluorescence of the first and optionally of the second fluorescent protein indicates absence of the first and/or the second antigen in the cell.

26. A method of promoting degradation of a target antigen in a cell, the method comprising expressing in the cell an internal fusion protein of claim 2 comprising a first nanobody directed against a first antigen, wherein the internal fusion protein is fused to a second nanobody directed against a second antigen that is to be targeted for degradation and wherein:
(a) in the presence of the first antigen, degradation of the second antigen is not promoted; and
(b) in the absence of the first antigen, degradation of the second antigen is promoted.

27. The method of claim 26, wherein a fluorescent protein is inserted into the first nanobody, and wherein:
(a) in the presence of the first antigen, the fluorescent protein emits fluorescence; and
(b) in the absence of the first antigen, the fluorescent protein does not emit fluorescence.

28. A method of regulating the expression of a protein of interest in a cell, the method comprising expressing in the cell an internal fusion protein of claim 2 comprising a nanobody directed against an antigen, wherein the internal fusion protein is fused to transcription factor that controls the expressing of the protein of interest, wherein:
(a) in the presence of the antigen, expression of the protein of interest is increased; and
(b) in the absence of the antigen, expression of the protein of interest is decreased.

29. The method of claim 28, wherein a fluorescent protein is inserted into the nanobody, and wherein:
(a) in the presence of the antigen, the fluorescent protein emits fluorescence; and
(b) in the absence of the antigen, the fluorescent protein does not emit fluorescence.

30. A method of regulating kinase activity in a cell, the method comprising expressing in the cell an internal fusion protein of claim 2 comprising a nanobody directed against an antigen, wherein the internal fusion protein is fused to a peptide that reduces the activity of a kinase and wherein:
(a) in the presence of the antigen, the activity of the kinase is reduced; and
(b) in the absence of the antigen, the activity of the kinase is not reduced.

31. The method of claim 30, wherein a fluorescent protein is inserted into the nanobody, and wherein:
(a) in the presence of the antigen, the fluorescent protein emits fluorescence; and
(b) in the absence of the antigen, the fluorescent protein does not emit fluorescence.


SEQUENCE LISTING (41 sequences)

SEQ ID NO: 1 - protein, 147 aa, Artificial Sequence
  Synthetic
MANLDKMLNTTVTEVRQFLQVDRVCVFQFEEDYSGVVVVEAVDDRWISILKTQVRDRYFMETRGEEYSHGRYQAIADIYTANLTECYRDLLTQFQVRAILAVPILQGKKLWGLLVAHQLAAPRQWQTWEIDFLKQQAVVVGIAIQQS

SEQ ID NO: 2 - protein, 147 aa, Artificial Sequence
  Synthetic
MANLDKMLNTTVTEVRQFLQVDRVCVFQFEEDYSGVVVVEAVDDRWISILKTQVRDCYFMETRGEEYSHGRYQAIADIYTANLTESYRDLLTQFQVRAILAVPILQGKKLWGLLVAHQLAAPRQWQTWEIDFLKQQAVVVGIAIQQS

SEQ ID NO: 3 - protein, 147 aa, Artificial Sequence
  Synthetic
MANLDKMLNTTVTEVRQFLQVDRVCVFQFEEDYSGVVVVEAVDDRWISILKTQVRDCYFMETRGEEYSHGRYQAIADIYTANLTESYRDLLTQFQVRAILAVPILQGKKLWGLFSAHQLAAPRQWQTWEIDFLKQQAVVVGIAIQQS

SEQ ID NO: 4 - protein, 147 aa, Artificial Sequence
  Synthetic
MANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQS

SEQ ID NO: 5 - protein, 147 aa, Artificial Sequence
  Synthetic
MANLDKMLNTIVTEVRQFLQVDRVCVFQFEEDYSGSVVVEAVDDRWNSILKTQVRDCYFMETRGEEYLHGRYQAIADIYQANLLESYRDLLGQFQVRAILAVPIIKGKKLWGLLVAHQLAAPRSWQTWEIEFLKQQAVVMGIAIQQS

SEQ ID NO: 6 - protein, 147 aa, Artificial Sequence
  Synthetic
MANLDKMLNTIVTEVRQFLQVDRLCVFKFEEDYSGNIIYEAVDDGWLSILKTHVRDCYFMETRGEEYLHGRYQAIADIHQANLAESYRDFLTQYQVRAIVAVPILKGKKLWGLFSAHQLAAPRSWQAWEIEFLKQQAVVMGIAIQQS

SEQ ID NO: 7 - protein, 147 aa, Artificial Sequence
  Synthetic
MANLDKMLNTTVTEVRKFLQADRVCVFKFEEDYSGTVSHEAVDDRWISILKTQVQDRCFMETRGEEYVHGRYQAIADIYTANLVECYRDLLIEFQVRAILAVPILQGKKLWGLLVAHQLAGPREWQTWEIDFLKQQAVVMGIAIQQS

SEQ ID NO: 8 - protein, 115 aa, Artificial Sequence [sequence omitted]

SEQ ID NO: 9 - protein, 121 aa, Artificial Sequence
  Synthetic
QLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVS

SEQ ID NO: 10 - protein, 120 aa, Artificial Sequence
  Synthetic
MAQVQLVESGGGLTQAGGSLRLSCATSGLIFSAFGMGWFRQAPGKEREFVGGINWRGSTNYGDFVKGRFTISRDNAKNTVYLQMNNLKPEDTAVYYCAARMVHKTEYDYWGEGTQVTVSS

SEQ ID NO: 11 - protein, 123 aa, Artificial Sequence
  Synthetic
MQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSS

SEQ ID NO: 12 - protein, 122 aa, Artificial Sequence
  Synthetic
MVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 13 - protein, 118 aa, Artificial Sequence
  Synthetic
MQVQLQESGGGLVQAGGSLRLSCKASGIIFSVYKMTWYRQAPGKERELVALITTNNNTMTVDSVKGRFTISRDNVQNTVYLEMNNLKPEDTAVYYCNANRGLAGPAYWGQGTQVTVSS

SEQ ID NO: 14 - protein, 115 aa, Artificial Sequence
  Synthetic
MDVQLQESGGGLVQAGGSLRLSCAASGSISRFNAMGWWRQAPGKEREFVARIVKGFDPVLADSVKGRFTISIDSAENTLALQMNRLKPEDTAVYYCFAALDTAYWGQGTQVTVSS

SEQ ID NO: 15 - protein, 121 aa, Artificial Sequence
  Synthetic
MDVQLQESGGGLVQPGGSLRLSCAASGNIVSIDAAGWFRQAPGKQREPVATILTGGATNYADSVKGRFTISRDNAKNTVYLQMNSLKPEDTAVYYCYAPMIYYGGRYSDYWGQGTQVTVSS

SEQ ID NO: 16 - protein, 118 aa, Artificial Sequence
  Synthetic
MQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSS

SEQ ID NO: 17 - protein, 120 aa, Artificial Sequence
  Synthetic
MQVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPASPAYGDYWGQGTQVTVSS

SEQ ID NO: 18 - protein, 274 aa, Artificial Sequence
  Synthetic
MDQVQLVESGGALVQPGGSLRLSCAASGFPVNRYSMRWYRQAPGGGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGGGSKEREWVAGMSSAGDRSSYEDSVKGRFTISRDDARNTVYLQMNSLKPEDTAVYYCNVNVGFEYWGQGTQVTVSS

SEQ ID NO: 19 - protein, 274 aa, Artificial Sequence
  Synthetic
MDQVQLVESGGALVQPGGSLRLSCAASGFPVNRYSMRWYRQAPGKEREWVAGMSSAGDRSSYEDSGGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGGGSVKGRFTISRDDARNTVYLQMNSLKPEDTAVYYCNVNVGFEYWGQGTQVTVSS

SEQ ID NO: 20 - protein, 274 aa, Artificial Sequence
  Synthetic
MDQVQLVESGGALVQPGGSLRLSCAASGFPVNRYSMRWYRQAPGKEREWVAGMSSAGDRSSYEDSVKGRFTISRDDARNTVYLQMNSLKPGGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGGGSEDTAVYYCNVNVGFEYWGQGTQVTVSS

SEQ ID NO: 21 - protein, 270 aa, Artificial Sequence
  Synthetic
MDQVQLVESGGALVQPGGSLRLSCAASGFPVNRYSMRWYRQAPGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGSKEREWVAGMSSAGDRSSYEDSVKGRFTISRDDARNTVYLQMNSLKPEDTAVYYCNVNVGFEYWGQGTQVTVSS

SEQ ID NO: 22 - protein, 270 aa, Artificial Sequence
  Synthetic
MDQVQLVESGGALVQPGGSLRLSCAASGFPVNRYSMRWYRQAPGKEREWVAGMSSAGDRSSYEDSGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGSVKGRFTISRDDARNTVYLQMNSLKPEDTAVYYCNVNVGFEYWGQGTQVTVSS

SEQ ID NO: 23 - protein, 270 aa, Artificial Sequence
  Synthetic
MDQVQLVESGGALVQPGGSLRLSCAASGFPVNRYSMRWYRQAPGKEREWVAGMSSAGDRSSYEDSVKGRFTISRDDARNTVYLQMNSLKPGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGSEDTAVYYCNVNVGFEYWGQGTQVTVSS

SEQ ID NO: 24 - protein, 278 aa, Artificial Sequence
  Synthetic
MDQVQLVESGGALVQPGGSLRLSCAASGFPVNRYSMRWYRQAPGGGSGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGSGGGSKEREWVAGMSSAGDRSSYEDSVKGRFTISRDDARNTVYLQMNSLKPEDTAVYYCNVNVGFEYWGQGTQVTVSS

SEQ ID NO: 25 - protein, 278 aa, Artificial Sequence
  Synthetic
MDQVQLVESGGALVQPGGSLRLSCAASGFPVNRYSMRWYRQAPGKEREWVAGMSSAGDRSSYEDSGGSGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGSGGGSVKGRFTISRDDARNTVYLQMNSLKPEDTAVYYCNVNVGFEYWGQGTQVTVSS

SEQ ID NO: 26 - protein, 278 aa, Artificial Sequence
  Synthetic
MDQVQLVESGGALVQPGGSLRLSCAASGFPVNRYSMRWYRQAPGKEREWVAGMSSAGDRSSYEDSVKGRFTISRDDARNTVYLQMNSLKPGGSGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGSGGGSEDTAVYYCNVNVGFEYWGQGTQVTVSS

SEQ ID NO: 27 - protein, 284 aa, Artificial Sequence
  Synthetic
MDQVQLVESGGALVQPGGSLRLSCAASGFPVNRYSMRWYRQAPGGGGGSGGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGGGSGGGGSKEREWVAGMSSAGDRSSYEDSVKGRFTISRDDARNTVYLQMNSLKPEDTAVYYCNVNVGFEYWGQGTQVTVSS

SEQ ID NO: 28 - protein, 284 aa, Artificial Sequence
  Synthetic
MDQVQLVESGGALVQPGGSLRLSCAASGFPVNRYSMRWYRQAPGKEREWVAGMSSAGDRSSYEDSGGGGSGGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGGGSGGGGSVKGRFTISRDDARNTVYLQMNSLKPEDTAVYYCNVNVGFEYWGQGTQVTVSS

SEQ ID NO: 29 - protein, 284 aa, Artificial Sequence
  Synthetic
MDQVQLVESGGALVQPGGSLRLSCAASGFPVNRYSMRWYRQAPGKEREWVAGMSSAGDRSSYEDSVKGRFTISRDDARNTVYLQMNSLKPGGGGSGGGGSMANLDKMLNTTVTEVRKFLQADRVCVFKFEEDYSGTVSHEAVDDRWISILKTQVQDRYFMETRGEEYVHGRYQAIADIYTANLVECYRDLLIEFQVRAILAVPILQGKKLWGLLVAHQLAGPREWQTWEIDFLKQQAVVMGIAIQQSGGGGSGGGGSEDTAVYYCNVNVGFEYWGQGTQVTVSS

SEQ ID NO: 30 - protein, 5 aa, Artificial Sequence
  Synthetic
GGGGS

SEQ ID NO: 31 - protein, 7 aa, Artificial Sequence
  Synthetic
GGSGGGS

SEQ ID NO: 32 - protein, 10 aa, Artificial Sequence
  Synthetic
GGGGSGGGGS

SEQ ID NO: 33 - protein, 18 aa, Artificial Sequence
  Synthetic
MANLDKMLNTTVTEVRKF

SEQ ID NO: 34 - protein, 21 aa, Artificial Sequence
  Synthetic
TWEIDFLKQQAVVMGIAIQQS

SEQ ID NO: 35 - protein, 16 aa, Artificial Sequence
  Synthetic
MGVKVLFALICIAVAE

SEQ ID NO: 36 - protein, 9 aa, Artificial Sequence
  Synthetic
GRTGRRNAI

SEQ ID NO: 37 - protein, 11 aa, Artificial Sequence
  Synthetic
RPKRPTTLNLF

SEQ ID NO: 38 - protein, 13 aa, Artificial Sequence
  Synthetic
SRLEEELRRRLTE

SEQ ID NO: 39 - protein, 9 aa, Artificial Sequence
  Synthetic
GRTGRRNAI

SEQ ID NO: 40 - protein, 11 aa, Artificial Sequence
  Synthetic
RPKRPTTLNLF

SEQ ID NO: 41 - protein, 20 aa, Artificial Sequence
  Synthetic
GGGGSGGGGSGGGGSGGGGS